(19) United States
(12) Patent Application Publication
(10) Pub. No.: US 2008/0280911 A1
(43) Pub. Date: Nov. 13, 2008

(54) Cinnoline Compounds as Inhibitors of Phosphodiesterase Type IV (Pde4)

(75) Inventors: Nicola Mary Aston, Hertfordshire (GB); John Edward Robinson, Hertfordshire (GB); Naimisha Trivedi, Hertfordshire (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: Glaxo Group Limited, Greenford, Middlesex (GB)

(21) Appl. No.: 12/090,491

(22) PCT Filed: Oct. 18, 2006
(86) PCT No.: PCT/GB06/03864

§ 371 Date: Apr. 17, 2008

(30) Foreign Application Priority Data
Oct. 21, 2005 (GB) 0521563.7

(51) Int. Cl. A61K 31/502; C07D 237/26; C07D 403/12; C07D 401/12; C07D 405/12; C07D 413/12; A61P 11/00

(52) U.S. Cl. 514/248; 544/235

(57) ABSTRACT

There are provided according to the invention novel compounds of formula (I)

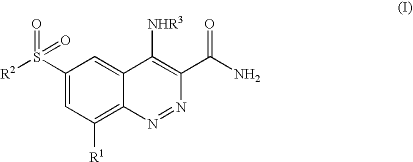

### FIELD OF THE INVENTION

The present invention relates to cinnoline compounds, processes for their preparation, intermediates usable in these processes, and pharmaceutical compositions containing the compounds. The invention also relates to the use of the cinnoline compounds in therapy, for example as inhibitors of phosphodiesterases and/or for the treatment and/or prophylaxis of diseases or conditions for which a PDE4 inhibitor is indicated such as inflammatory and/or allergic diseases including chronic obstructive pulmonary disease (COPD), asthma, rheumatoid arthritis or allergic rhinitis.

### BACKGROUND TO THE INVENTION

International Application WO2004/103998 (Glaxo Group Limited) describes quinoline compounds of the formula (A):

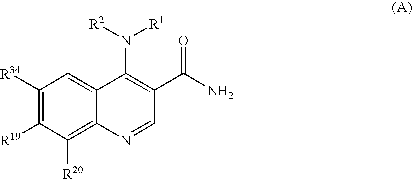

in which R¹, R², R¹⁹, R²⁰ and R³⁴ are as defined in the International Application; which inhibit phosphodiesterase type IV (PDE4).

It is desirable to find further, new compounds which bind to, and preferably inhibit, phosphodiesterase type IV (PDE4).

### SUMMARY OF THE INVENTION

According to the invention there is provided a compound of formula (I):

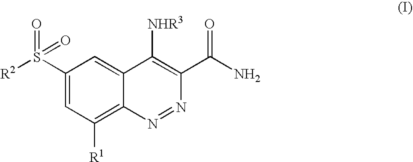

wherein:

R¹ is hydrogen, fluoro or methyl;

R² is C₁₋₆alkyl; and

R³ is phenyl or pyridinyl, each of which is unsubstituted or substituted with one or two substituents which may be the same or different selected from fluoro, chloro, cyano, methyl or methoxy; phenyl fused to a 5-membered saturated ring containing one oxygen atom and optionally substituted on the phenyl ring with one fluoro; 3,4-dimethyl isoxazolyl; or N—C₁₋₂alkyl-pyrazolyl; or a pharmaceutically acceptable salt or solvate thereof.

In a second aspect of the present invention, there is provided a compound of formula (I), or a pharmaceutically acceptable salt or solvate thereof for use in therapy, in particular in the treatment of diseases or conditions for which a PDE4 inhibitor is indicated, more particularly inflammatory and/or allergic diseases.

In a third aspect of the present invention, there is provided a pharmaceutical composition comprising a compound of formula (I) or a pharmaceutically acceptable salt or solvate thereof and one or more of pharmaceutically acceptable carriers, diluents and excipients.

In a fourth aspect of the present invention, there is provided a method of treating diseases or conditions for which a PDE4 inhibitor is indicated, more particularly inflammatory and/or allergic diseases comprising administering a compound of formula (I) or a pharmaceutically acceptable salt or solvate thereof.

In a fifth aspect of the present invention, there is provided the use of a compound of formula (I), or a pharmaceutically acceptable salt or solvate thereof in the manufacture of a medicament for the treatment of diseases or conditions for which a PDE4 inhibitor is indicated, more particularly inflammatory and/or allergic diseases.

### DETAILED DESCRIPTION OF THE INVENTION

The term “PDE 4 inhibitor” is used to mean a compound which binds to and preferably inhibits PDE 4 activity.

The term “diseases or conditions for which a PDE4 inhibitor is indicated”, is used to mean any disease state mediated or modulated by PDE 4 mechanisms, in particular inflammatory and/or allergic diseases including asthma, chronic bronchitis, emphysema, urticaria, allergic rhinitis (seasonal or perennial), vasomotor rhinitis, nasal polyps, allergic conjunctivitis, vernal conjunctivitis, occupational conjunctivitis, infective conjunctivitis, eosinophilic syndromes, eosinophilic granuloma, rheumatoid arthritis, chronic obstructive pulmonary disease (COPD) septic shock, ulcerative colitis, Crohn's disease, irritable bowel disease, irritable bowel syndrome, reperfusion injury of the myocardium and brain, chronic glomerulonephritis, endotoxic shock, adult respiratory distress syndrome, multiple sclerosis, Parkinsons disease, schizophrenia or memory impairment (including Alzheimer's disease), pain, depression, or allergic or inflammatory skin diseases such as psoriasis or atopic dermatitis.

As used herein, “a compound of the invention” means a compound of formula (I) or a salt or solvate thereof.

The compounds of the invention may have the ability to crystallize in more than one form, a characteristic, which is known as polymorphism, and it is understood that such polymorphic forms (“polymorphs”) are within the scope of formula (I). Polymorphism generally can occur as a response to changes in temperature or pressure or both and can also result from variations in the crystallization process. Polymorphs can be distinguished by various physical characteristics known in the art such as x-ray diffraction patterns, solubility and melting point.

Certain of the compounds described herein may contain one or more chiral atoms so that optical isomers, eg—enantiomers or disastereoisomers may be formed. Accordingly, the present invention encompasses all isomers of the compounds of formula (I) whether as individual isomers isolated such as to be substantially free of the other isomer (i.e. pure) or as mixtures isolated such as to be substantially free of the other isomer (i.e. pure) or as mixtures thereof (i.e. racemates and racemic mixtures). An individual isomer isolated such as to be substantially free of the other isomer (i.e. pure) may be isolated such that less than 10%, particularly less than about 1%, for example less than about 0.1% of the other isomer is present.

It is also noted that the compounds of the invention may form tautomers. It is understood that all tautomers and mixtures of tautomers of the compounds of the present invention are included within the scope of the compounds of the present invention.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the term “solvate” refers to a complex of variable stoichiometry formed by a solute (in this invention, a compound of the invention) and a solvent. Such solvents for the purpose of the invention may not interfere with the biological activity of the solute. Examples of suitable solvents include, but are not limited to, water, acetone, methanol, ethanol and acetic acid. In one aspect the solvent used is a pharmaceutically acceptable solvent. Examples of suitable pharmaceutically acceptable solvents include water, ethanol and acetic acid. In one aspect the solvent is water.

As used herein, the term “alkyl” refers to straight or branched hydrocarbon chains containing the specified number of carbon atoms. For example, C₁₋₆alkyl means a straight or branched alkyl chain containing at least 1, and at most 6, carbon atoms. Examples of “alkyl” as used herein include, but are not limited to, methyl, ethyl, n-propyl, iso-propyl, n-butyl, sec-butyl, t-butyl, pentyl and hexyl.

As used herein, the term “cyano” refers to the group—CN.

In one aspect, there is provided a compound of formula (I) which is of formula (Ia):

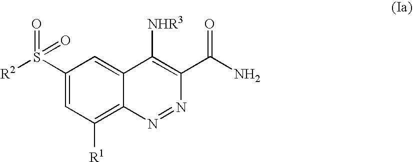

wherein:

R¹ is hydrogen, fluoro or methyl;

R² is C₁₋₆alkyl; and

R³ is selected from phenyl or pyridinyl each of which is unsubstituted or substituted with one substitutent selected from fluoro, chloro or cyano; phenyl substituted with two substituents which may be the same or different selected from fluoro, chloro or methoxy; phenyl fused to a 5-membered saturated ring containing one oxygen atom and optionally substituted on the phenyl ring with one fluoro; or N—C₁₋₂alkyl-pyrazolyl; or a pharmaceutically acceptable salt or solvate thereof.

In one embodiment R¹ in formula (I) is H or methyl.

In one embodiment R² in formula (I) is methyl, ethyl, n-propyl and t-butyl.

In one embodiment R³ in formula (i) is 3-cyanophenyl, 2,3-difluorophenyl, 3,5-difluorophenyl, 2-fluoro-3-chlorophenyl, 4-fluoro-3-(methyloxy)phenyl, 5-cyano-3-pyridinyl, 5-fluoro-3-pyridinyl, 5-chloro-3-pyridinyl, 7-fluoro-2,3-dihydro-1-benzofuran-4-yl, 1-ethyl-1H-pyrazol-5-yl, 3,4-dimethyl-5-isoxazolyl or 6-fluoro-5-methyl-3-pyridinyl.

In one embodiment R³ in formula (I) is 3-cyanophenyl, 2,3-difluorophenyl, 3,5-difluorophenyl, 2-fluoro-3-chlorophenyl, 4-fluoro-3-(methyloxy)phenyl, 5-cyano-3-pyridinyl, 5-fluoro-3-pyridinyl, 5-chloro-3-pyridinyl, 7-fluoro-2,3-dihydro-1-benzofuran-4-yl or 1-ethyl-1H-pyrazol-5-yl.

In a further embodiment R³ in formula (I) is 3-cyanophenyl, 3,5-difluorophenyl, 5-fluoro-3-pyridinyl, 1-ethyl-1H-pyrazol-5-yl, 5-chloro-3-pyridinyl or 3,4-dimethyl-5-isoxazolyl.

In a further embodiment R³ in formula (I) is 3-cyanophenyl, 3,5-difluorophenyl, 5-fluoro-3-pyridinyl, 5-chloro-3-pyridinyl or 1-ethyl-1H-pyrazol-5-yl.

In an embodiment of the invention, R¹ in formula (I) is methyl.

In an embodiment of the invention, R² in formula (I) is methyl.

In an embodiment of the invention, R³ in formula (I) is 3-cyanophenyl.

While the embodiments for each variable have generally been listed above separately for each variable this invention includes those compounds in which several or each embodiment in formula (I) is selected from each of the embodiments listed above. Therefore, this invention is intended to include all combinations of embodiments for each variable described hereinabove including salts and solvates thereof.

Particular compounds according to the invention include:
4-[(3-cyanophenyl)amino]-8-methyl-6-(methylsulfonyl)-3-cinnolinecarboxamide;
6-[(1,1-dimethylethyl)sulfonyl]-4-[(1-ethyl-1H-pyrazol-5-yl)amino]-8-methyl-3-cinnolinecarboxamide;
4-[(1-ethyl-1H-pyrazol-5-yl)amino]-6-(ethylsulfonyl)-8-methyl-3-cinnolinecarboxamide;
4-[(3-cyanophenyl)amino]-6-(methylsulfonyl)-3-cinnolinecarboxamide;
4-[(7-fluoro-2,3-dihydro-1-benzofuran-4-yl)amino]-6-(methylsulfonyl)-3-cinnolinecarboxamide;
4-[(3-chloro-2-fluorophenyl)amino]-6-(methylsulfonyl)-3-cinnolinecarboxamide;
4-[(3,5-difluorophenyl)amino]-6-(methylsulfonyl)-3-cinnolinecarboxamide;
4-[(5-cyano-3-pyridinyl)amino]-6-(methylsulfonyl)-3-cinnolinecarboxamide;
4-[(1-ethyl-1H-pyrazol-5-yl)amino]-6-(methylsulfonyl)-3-cinnolinecarboxamide;
4-[(5-fluoro-3-pyridinyl)amino]-6-(methylsulfonyl)-3-cinnolinecarboxamide;
6-(ethylsulfonyl)-4-[(5-fluoro-3-pyridinyl)amino]-3-cinnolinecarboxamide;
6-[(1,1-dimethylethyl)sulfonyl]-4-[(5-fluoro-3-pyridinyl)amino]-3-cinnolinecarboxamide;
4-[(5-chloro-3-pyridinyl)amino]-6-(methylsulfonyl)-3-cinnolinecarboxamide;
4-[(5-chloro-3-pyridinyl)amino]-6-(ethylsulfonyl)-3-cinnolinecarboxamide;
4-[(5-cyano-3-pyridinyl)amino]-6-(ethylsulfonyl)-3-cinnolinecarboxamide;
4-[(1-ethyl-1H-pyrazol-5-yl)amino]-6-(ethylsulfonyl)-3-cinnolinecarboxamide;
4-[(5-chloro-3-pyridinyl)amino]-8-methyl-6-(methylsulfonyl)-3-cinnolinecarboxamide;
4-[(3,4-dimethyl-5-isoxazolyl)amino]-8-methyl-6-(methylsulfonyl)-3-cinnolinecarboxamide;
4-[(5-chloro-3-pyridinyl)amino]-6-[( ,1-dimethylethyl)sulfonyl]-3-cinnolinecarboxamide;
4-[(5-cyano-3-pyridinyl)amino]-6-[(1,1-dimethylethyl)sulfonyl]-3-cinnolinecarboxamide;
6-[(1,1-dimethylethyl)sulfonyl]-4-[(1-ethyl-1H-pyrazol-5-yl)amino]-3-cinnolinecarboxamide;
4-[(6-fluoro-5-methyl-3-pyridinyl)amino]-8-methyl-6-(methylsulfonyl)-3-cinnolinecarboxamide;
6-(ethylsulfonyl)-4-[(5-fluoro-3-pyridinyl)amino]-8-methyl-3-cinnolinecarboxamide;
4-[(5-cyano-3-pyridinyl)amino]-6-(ethylsulfonyl)-8-methyl-3-cinnolinecarboxamide;
4-{[4-fluoro-3-(methyloxy)phenyl]amino}-8-methyl-6-(methylsulfonyl)-3-cinnolinecarboxamide;
4-[(5-fluoro-3-pyridinyl)amino]-8-methyl-6-(methylsulfonyl)-3-cinnolinecarboxamide;
4-[(5-cyano-3-pyridinyl)amino]-8-methyl-6-(methylsulfonyl)-3-cinnolinecarboxamide;
4-[(1-ethyl-1H-pyrazol-5-yl)amino]-8-methyl-6-(methylsulfonyl)-3-cinnolinecarboxamide;
4-[(2,3-difluorophenyl)amino]-8-methyl-6-(methylsulfonyl)-3-cinnolinecarboxamide;
4-[(3-chloro-2-fluorophenyl)amino]-8-methyl-6-(methylsulfonyl)-3-cinnolinecarboxamide;
4-[(7-fluoro-2,3-dihydro-1-benzofuran-4-yl)amino]-8-methyl-6-(methylsulfonyl)-3-cinnolinecarboxamide;
4-[(3,5-difluorophenyl)amino]-8-methyl-6-(methylsulfonyl)-3-cinnolinecarboxamide;
4-[(1-ethyl-1H-pyrazol-5-yl)amino]-8-methyl-6-(propylsulfonyl)-3-cinnolinecarboxamide;
6-[(1,1-dimethylethyl)sulfonyl]-4-[(5-fluoro-3-pyridinyl)amino]-8-methyl-3-cinnolinecarboxamide;
4-[(5-chloro-3-pyridinyl)amino]-6-(ethylsulfonyl)-8-methyl-3-cinnolinecarboxamide;
6-(ethylsulfonyl)-8-fluoro-4-[(5-fluoro-3-pyridinyl)amino]-3-cinnolinecarboxamide;
8-fluoro-4-[(5-fluoro-3-pyridinyl)amino]-6-(methylsulfonyl)-3-cinnolinecarboxamide;
4-[(1-ethyl-1H-pyrazol-5-yl)amino]-6-(ethylsulfonyl)-8-methyl-3-cinnolinecarboxamide;
   and pharmaceutically acceptable salts and solvates thereof.

Specific examples which may be mentioned include:
4-[(3-cyanophenyl)amino]-8-methyl-6-(methylsulfonyl)-3-cinnolinecarboxamide;
4-[(3,5-difluorophenyl)amino]-6-(methylsulfonyl)-3-cinnolinecarboxamide;
4-[(5-fluoro-3-pyridinyl)amino]-6-(methylsulfonyl)-3-cinnolinecarboxamide;
6-[(1,1-dimethylethyl)sulfonyl]-4-[(5-fluoro-3-pyridinyl)amino]-3-cinnolinecarboxamide;
4-[(5-chloro-3-pyridinyl)amino]-6-(methylsulfonyl)-3-cinnolinecarboxamide;
4-[(5-chloro-3-pyridinyl)amino]-8-methyl-6-(methylsulfonyl)-3-cinnolinecarboxamide;
4-[(3,4-dimethyl-5-isoxazolyl)amino]-8-methyl-6-(methylsulfonyl)-3-cinnolinecarboxamide;
4-[(1-ethyl-1H-pyrazol-5-yl)amino]-6-(ethylsulfonyl)-8-methyl-3-cinnolinecarboxamide;
   and pharmaceutically acceptable salts and solvates thereof.

In one embodiment there is provided:

4-[(3-cyanophenyl)amino]-8-methyl-6-(methylsulfonyl)-3-cinnolinecarboxamide

and pharmaceutically acceptable salts and solvates thereof.

In a further embodiment there is provided:

4-[(3-cyanophenyl)amino]-8-methyl-6-(methylsulfonyl)-3-cinnolinecarboxamide.

The compounds of the present invention may be in the form of and/or may be administered as a pharmaceutically acceptable salt. For a review on suitable salts see Berge et al, J. Pharm. Sci. 1977, 66, 1-19.

Typically, the salts of the present invention are pharmaceutically acceptable salts. Salts encompassed within the term “pharmaceutically acceptable salts” refer to non-toxic salts of the compounds of formula (I).

Suitable pharmaceutically acceptable salts can include acid addition salts.

A pharmaceutically acceptable acid addition salt can be formed by reaction of a compound of formula (I) with a suitable inorganic or organic acid (such as hydrobromic, hydrochloric, sulfuric, nitric, phosphoric, p-toluenesulfonic, benzenesulfonic, methanesulfonic, ethanesulfonic, naphthalenesulfonic such as 2-naphthalenesulfonic), optionally in a suitable solvent such as an organic solvent, to give the salt which is usually isolated for example by crystallisation and filtration. A pharmaceutically acceptable acid addition salt of a compound of formula (I) can comprise or be for example a hydrobromide, hydrochloride, sulfate, nitrate, phosphate, p-toluenesulfonate, benzenesulfonate, methanesulfonate, ethanesulfonate, naphthalenesulfonate (e.g. 2-naphthalenesulfonate) salt.

Other non-pharmaceutically acceptable salts, e.g. trifluoroacetates, may be used, for example in the isolation of compounds of the invention, and are included within the scope of this invention.

The invention includes within its scope all possible stoichiometric and non-stoichiometric forms of the compounds of the invention.

The compounds of the invention may be made by a variety of methods, including standard chemistry. Any previously defined variable will continue to have the previously defined meaning unless otherwise indicated. Illustrative general synthetic methods are set out below and then specific compounds of the invention are prepared in the working Examples.

Compounds of formula (I) and (Ia), wherein R¹, R² and R³ are as hereinbefore defined, may be prepared as outlined in Scheme 1, from the intermediate of formula (V).

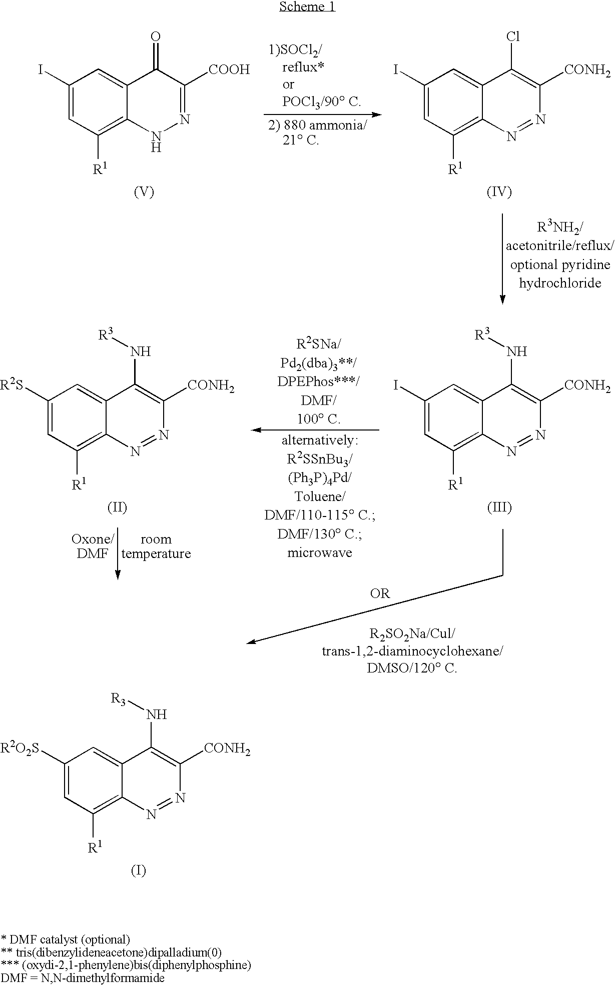

For example, a compound of formula (I) wherein R¹ is CH₃, R² is methyl and R³ is 3-cyanophenyl may be prepared from intermediate (V) by Scheme 1(a):

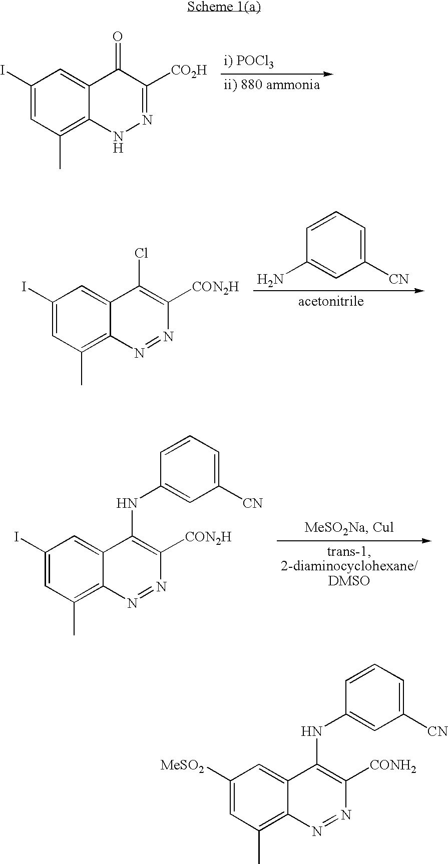

Alternatively, the order of introduction of the R² and R³ substituents may be reversed, as shown in Scheme 2:

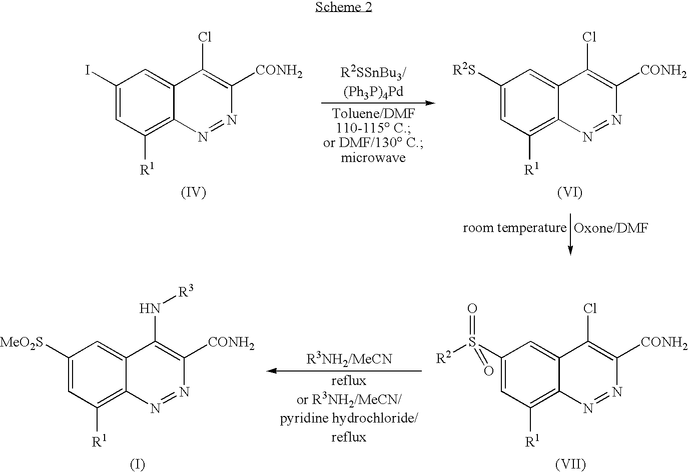

The second and third reaction in Scheme 2 may be performed in either order.

Intermediates of formula (V) may be prepared according to Scheme 3:

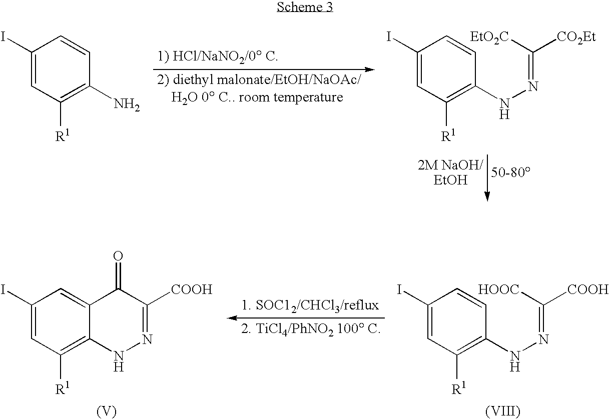

Intermediates of formula (V) may also be prepared from Intermediates of formula (VII) by the process shown in Scheme 4:

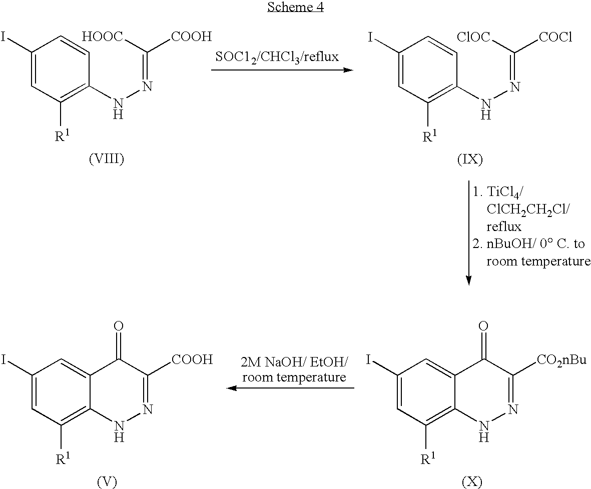

The Intermediate (V) in which R¹ is hydrogen may also be prepared according to Scheme 5:

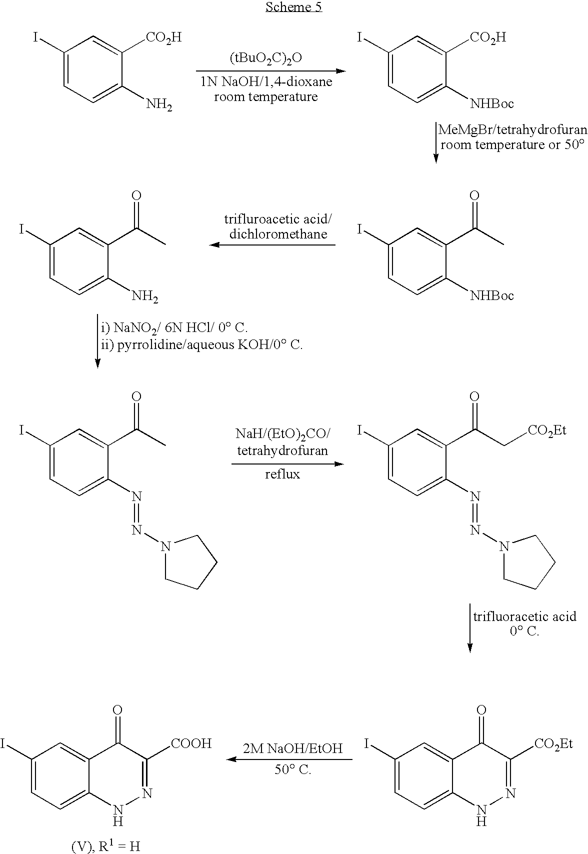

The compound of formula (V) where R¹ is methyl may be prepared according to Scheme 6:

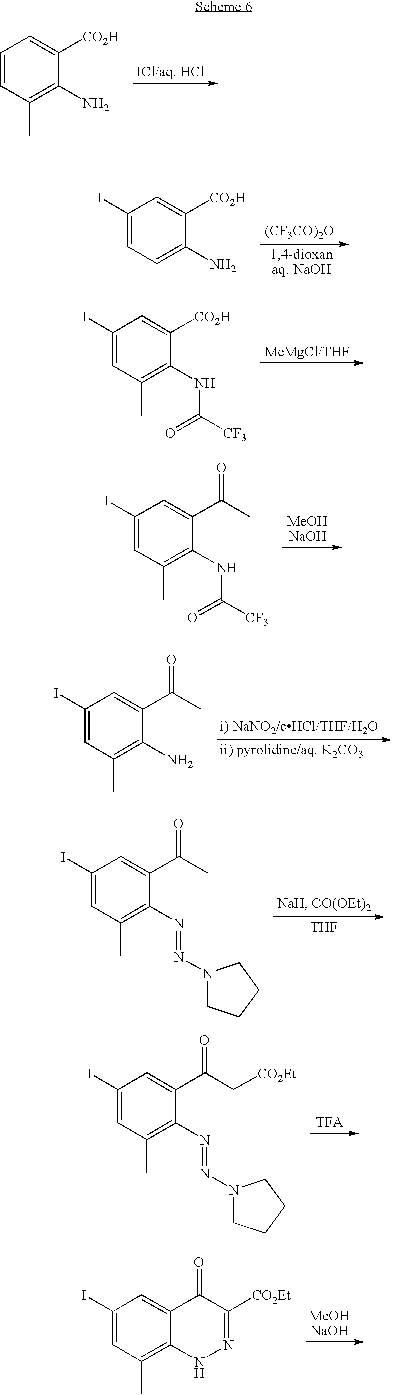

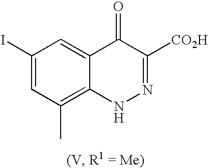

The Intermediate (V) where R¹ is fluorine may be prepared according to Scheme 7:

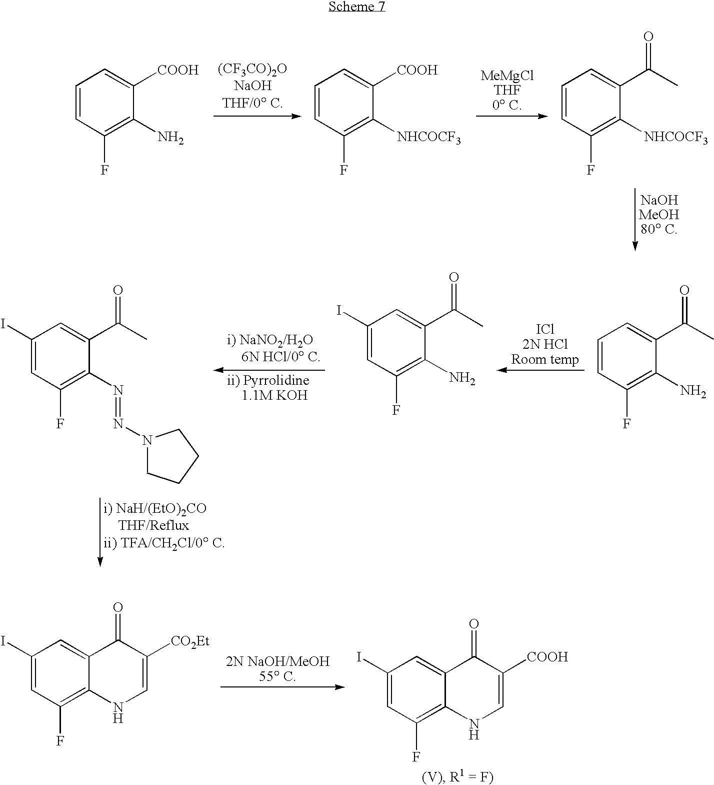

A compound of formula (I) may be prepared by:

(a) treating a corresponding Intermediate (II):

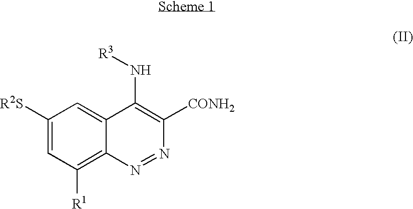

in which R¹, R² and R³ are as hereinbefore defined;

with an oxidising agent capable of oxidising a thioether to a sulphone, for instance oxone, in a suitable solvent such as N,N-dimethylformamide, at a suitable temperature such as room temperature; or

(b) treating an Intermediate (VII):

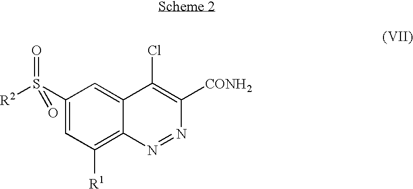

in which R¹, and R² are as hereinbefore defined;

with an amine R³NH₂, in a suitable solvent such as acetonitrile, at an elevated temperature, for instance under reflux where necessary in the presence of a suitable acidic catalyst, such as pyridine hydrochloride; or

c) treating a compound of formula (III)

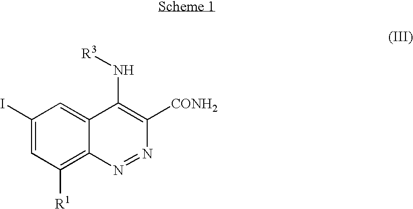

in which R¹ and R³ are as hereinbefore defined;

with a suitable alkyl sulphinic acid salt, such as a sodium alkanesulphinate (for example sodium methanesulphinate), in the presence of a suitable catalyst such as a copper catalyst, for example copper (1) iodide, and a suitable ligand such as trans-1,2-diaminocyclohexane, in a suitable solvent such as dimethylsulphoxide, at an elevated temperature such as 120° C.

Intermediate (II) may be prepared by a process which comprises treating a compound of formula (III):

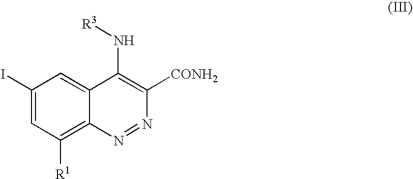

in which R¹ and R³ are as hereinbefore defined;

with a metal thioalkoxide R²SX in which X is an alkali metal salt such as sodium, in the presence of a suitable catalyst such as a palladium catalyst, e.g. tris(dibenzylidenacetone)dipalladium(0) and a suitable ligand such as a phosphine ligand, e.g. (oxydi-2,1-phenylene)bis(diphenylphosphine), in a suitable solvent such as N,N-dimethylformamide, optionally in the presence of a suitable base such as sodium tert-butoxide, at an elevated temperature, for instance about 100° C.; or

an (alkylthio)stannane such as an tributyl(alkylthio)stannane of formula R²SSnBu₃, wherein R² is as defined above, in the presence of a suitable catalyst such as a palladium catalyst, e.g. tetrakis(triphenylphosphine)palladium(0), in a suitable solvent such as a mixture of toluene and N,N-dimethylformamide, at an elevated temperature, for instance in the range 110 to 115° C. Alternative conditions for the reaction of a compound of formula (III) with an tributyl(alkylthio)stannane of formula R²SSnBu₃ include heating under microwave irradiation in a suitable solvent such as N,N-dimethylformamide at a suitable temperature, such as 130° C.

Intermediate (III) may be prepared by a process which comprises treating a compound of formula (IV):

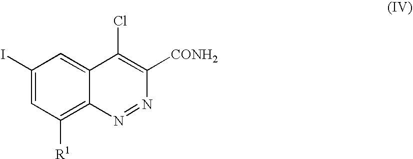

in which R¹ is as hereinbefore defined;

with an amine R³NH₂ in which R³ is as hereinbefore defined,

in a suitable solvent such as acetonitrile, and at an elevated temperature, for instance under reflux where necessary in the presence of a suitable acidic catalyst, such as pyridine hydrochloride.

A compound of formula (IV) may be prepared by a process which comprises treating a compound of formula (V) as hereinbefore defined with:

a chlorinating agent such as thionyl chloride, optionally in the presence of a catalyst such as N,N-dimethylformamide, at an elevated temperature, for instance under reflux; or

phosphorus oxychloride, at an elevated temperature, for instance at about 90° C.;

followed by 880 ammonia, optionally in the presence of a solvent such as 1,4-dioxane or toluene at about room temperature.

Intermediate (V) as hereinbefore defined may be obtained according to the sequence of scheme 4, in particular, the key second step whereby the initially formed product of cyclising the compound of formula (IX):

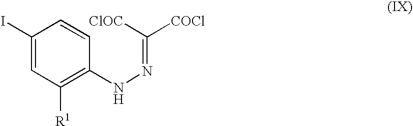

(using titanium tetrachloride in a suitable solvent such as 1,2-dichloroethane, at an elevated temperature, for instance, reflux) is then reacted with n-butanol, at a temperature in the range 0° C. to room temperature, such that the product is isolated as the butyl ester of formula (X), which can then be hydrolysed to the corresponding free acid of formula (V).

Intermediate (VII) may be prepared by a process which comprises treating a compound of formula (VI):

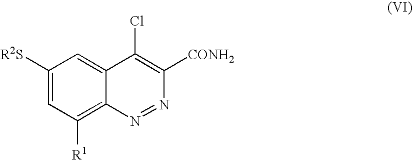

in which R¹, and R² are as hereinbefore defined;

with an oxidising agent capable of oxidising a thioether to a sulphone, as hereinbefore defined for the oxidation of a compound of formula (II).

Intermediate (VI) may be prepared by a process which comprises treating Intermediate (IV) (as hereinbefore defined) with an (alkylthio)stannane such as an tributyl(alkylthio)stannane of formula R²SSnBu₃, wherein R² is as defined above, in the presence of a suitable catalyst such as a palladium catalyst, e.g. tetrakis(triphenylphosphine)palladium(0), as hereinbefore defined for the preparation of a compound of formula (II).

Intermediates of formula (II) are novel and useful as intermediates in preparing compounds of formula (I). Accordingly, in a further aspect, the present invention provides for a compound of formula (II):

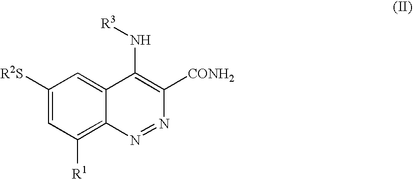

in which R¹, R² and R³ are as hereinbefore defined.

Intermediates of formula (VII) are novel and useful as intermediates in preparing compounds of formula (I). Accordingly, in a further aspect, the present invention provides for a compound of formula (VII):

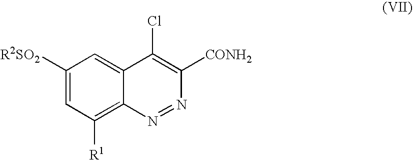

in which R¹, and R² are as hereinbefore defined.

Intermediates of formulae (III), (IV), (V), (VI), (VII), (IX) and (X) are also novel and useful as intermediates in preparing compounds of formula (I) and accordingly form a further aspect of the present invention.

Compounds of formula (I) may also be prepared by a process of deprotection of protected derivatives of compounds of formula (I). Examples of suitable protecting groups and the means for their removal can be found in T. W. Greene and P. G. M. Wuts ‘Protective Groups in Organic Synthesis’ (3^{rd} Ed., J. Wiley and Sons, 1999).

The compounds of formula (I) and salts and solvates thereof are believed to be inhibitors of PDE4 activity, and thus be potentially useful in the treatment of diseases or conditions for which a PDE4 compound is indicated, particularly inflammatory and/or allergic diseases.

The present invention thus provides a compound of formula (I) or a pharmaceutically acceptable salt or solvate thereof for use in therapy. The compound or salt or solvate thereof can be for use in the treatment and/or prophylaxis of any diseases or conditions for which a PDE4 inhibitor is indicated particularly inflammatory and/or allergic diseases.

Also provided is the use of a compound of formula (I) or a pharmaceutically acceptable salt or solvate thereof in the manufacture of a medicament (e.g. pharmaceutical composition) for the treatment of diseases or conditions for which a PDE4 inhibitor is indicated, particularly inflammatory and/or allergic diseases.

Also provided is a method of treating diseases or conditions for which a PDE4 inhibitor is indicated, particularly inflammatory and/or allergic diseases which comprises administering a compound of formula (I) or a pharmaceutically acceptable salt or solvate thereof.

“Therapy” and “treatment” may include treatment and/or prophylaxis.

Phosphodiesterase 4 inhibitors are believed to be useful in the treatment and/or prophylaxis of a variety of diseases, especially inflammatory and/or allergic diseases for example: asthma, chronic bronchitis, emphysema, urticaria, allergic rhinitis (seasonal or perennial), vasomotor rhinitis, nasal polyps, allergic conjunctivitis, vernal conjunctivitis, occupational conjunctivitis, infective conjunctivitis, eosinophilic syndromes, eosinophilic granuloma, rheumatoid arthritis, chronic obstructive pulmonary disease (COPD) including chronic bronchitis and emphysema, septic shock, ulcerative colitis, Crohn's disease, reperfusion injury of the myocardium and brain, chronic glomerulonephritis, endotoxic shock, adult respiratory distress syndrome, multiple sclerosis, or memory impairment (including Alzheimer's disease), pain or depression. PDE4 inhibitors may also be of use in the treatment and/or prophylaxis of an inflammatory and/or allergic skin disease, such as atopic dermatitis or psoriasis.

In the treatment and/or prophylaxis, the inflammatory and/or allergic disease is preferably chronic obstructive pulmonary disease (COPD) including chronic bronchitis and emphysema, asthma, rheumatoid arthritis, or allergic rhinitis, atopic dermatitis or psoriasis in a mammal (e.g. human). More preferably, the treatment and/or prophylaxis is of COPD including chronic bronchitis and emphysema, or asthma or allergic rhinitis in a mammal (e.g. human). PDE4 inhibitors are thought to be effective in the treatment of asthma (e.g. see M. A. Giembycz, Drugs, February 2000, 59(2), 193-212; Z. Huang et al., Current Opinion in Chemical Biology, 2001, 5, 432-438; and refs cited therein) and COPD (e.g. see S. L. Wolda, Emerging Drugs, 2000, 5(3), 309-319; Z. Huang et al., Current Opinion in Chemical Biology, 2001, 5, 432-438; and refs cited therein). COPD is often characterised by the presence of airflow obstruction due to chronic bronchitis and/or emphysema (S. L. Wolda, Emerging Drugs, 2000, 5(3), 309-319).

PDE4 inhibitors are thought to be effective in the treatment of allergic rhinitis (e.g. see B. M. Schmidt et al., J. Allergy & Clinical Immunology, 108(4), 2001, 530-536).

PDE4 inhibitors are thought to be effective in the treatment of rheumatoid arthritis and multiple sclerosis (e.g. see H. J. Dyke et al., Expert Opinion on Investigational Drugs, January 2002, 11(1), 1-13; C. Burnouf et al., Current Pharmaceutical Design, 2002, 8(14), 1255-1296; and A. M. Doherty, Current Opinion Chem. Biol., 1999, 3(4), 466-473; and refs cited therein). See e.g. A. M. Doherty, Current Opinion Chem. Biol., 1999, 3(4), 466-473 and refs cited therein for atopic dermatitis use.

PDE4 inhibitors have been suggested as having analgesic properties and thus being effective in the treatment of pain (A. Kumar et al., Indian J. Exp. Biol., 2000, 38(1), 26-30).

In the invention, the treatment and/or prophylaxis can be of cognitive impairment e.g. cognitive impairment in a neurological disorder such as Alzheimer's disease. For example, the treatment and/or prophylaxis may comprise cognitive enhancement e.g. in a neurological disorder. See for example: H. T. Zhang et al. in: Psychopharmacology, June 2000, 150(3), 311-316 and Neuropsychopharmacology, 2000, 23(2), 198-204; and T. Egawa et al., Japanese J. Pharmacol., 1997, 75(3), 275-81. PDE4 inhibitors such as rolipram have been suggested as having antidepressant properties (e.g. J. Zhu et al., CNS Drug Reviews, 2001, 7(4), 387-398; O'Donnell, Expert Opinion on Investigational Drugs, 2000, 9(3), 621-625; and H. T. Zhang et al., Neuropsychopharmacology, October 2002, 27(4), 587-595).

While it is possible that for use in therapy, a compound of formula (I) as well as pharmaceutically acceptable salts and solvates thereof may be administered as the raw chemical, it is possible to present the active ingredient as a pharmaceutical composition.

The present invention therefore provides in a further aspect a pharmaceutical composition comprising a compound of formula (I) or a pharmaceutically acceptable salt or solvate thereof and one or more or pharmaceutically acceptable carriers, diluents and/or excipients. The compounds of the formula (I) and pharmaceutically acceptable salts, or solvates thereof, are as described above. The carrier(s), diluent(s) or excipient(s) must be acceptable in the sense of being compatible with the other ingredients of the composition and not deleterious to the recipient thereof. In accordance with another aspect of the invention there is also provided a process for the preparation of a pharmaceutical composition including admixing a compound of the formula (I), or pharmaceutically acceptable salts, or solvates thereof, with one or more pharmaceutically acceptable carriers, diluents or excipients. The pharmaceutical composition can be for use in the treatment and/or prophylaxis of any of the conditions described herein.

Since the compounds of formula (I) are intended for use in pharmaceutical compositions it will be readily understood that they are each preferably provided in substantially pure form, for example, at least 60% pure, more suitably at least 75% pure and preferably at least 85% pure, especially at least 98% pure (% in a weight for weight basis).

In one embodiment, the compound of formula (I) or the pharmaceutically acceptable salt or solvate thereof can optionally be in a particle-size-reduced form, for example obtained or obtainable by micronisation.

Pharmaceutical compositions may be presented in unit dose forms containing a predetermined amount of active ingredient per unit dose. Preferred unit dosage compositions are those containing a daily dose or sub-dose, or an appropriate fraction thereof, of an active ingredient. Such unit doses may therefore be administered more than once a day. Preferred unit dosage compositions are those containing a daily dose or sub-dose (for administration more than once a day), as herein above recited, or an appropriate fraction thereof, of an active ingredient. Furthermore, such pharmaceutical compositions may be prepared by any of the methods well known in the pharmacy art.

Pharmaceutical compositions may be adapted for administration by any appropriate route, for example by the oral (including buccal or sublingual), rectal, inhaled, intranasal, topical (including buccal, sublingual or transdermal), vaginal or parenteral (including subcutaneous, intramuscular, intravenous or intradermal) route. Such compositions may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders suitable for incorporating into tablets or capsules may be prepared by reducing the compound to a suitable fine size (e.g. by micronisation) and mixing with a similarly prepared pharmaceutical carrier such as an edible carbohydrate, as, for example, starch or mannitol. Flavoring, preservative, dispersing and coloring agent can also be present.

Capsules may be made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, glidants, lubricants, sweetening agents, flavours, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit compositions for oral administration can be microencapsulated. The formulation can also be prepared to prolong or sustain the release as for example by coating or embedding particulate material in polymers, wax or the like.

The compounds of the invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Pharmaceutical compositions adapted for transdermal administration may be presented as discrete patches intended to remain in intimate contact with the epidermis of the recipient for a prolonged period of time.

Pharmaceutical compositions adapted for topical administration may be formulated as ointments, creams, suspensions, lotions, powders, solutions, pastes, gels, sprays, aerosols or oils.

For treatments of the eye or other external tissues, for example mouth and skin, the compositions are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical compositions adapted for topical administrations to the eye include eye drops wherein the active ingredient is dissolved or suspended in a suitable carrier, especially an aqueous solvent.

Pharmaceutical compositions adapted for topical administration in the mouth include lozenges, pastilles and mouth washes.

Pharmaceutical compositions adapted for rectal administration may be presented as suppositories or as enemas.

Dosage forms for nasal or inhaled administration may conveniently be formulated as aerosols, solutions, suspensions, gels or dry powders.

For compositions suitable and/or adapted for inhaled administration, it is preferred that the compound of the invention is in a particle-size-reduced form, and more preferably the size-reduced form is obtained or obtainable by micronisation. The preferable particle size of the size-reduced (e.g. micronised) compound or salt or solvate is defined by a D50 value of about 0.5 to about 10 microns (for example as measured using laser diffraction).

Aerosol formulations, e.g. for inhaled administration, can comprise a solution or fine suspension of the active substance in a pharmaceutically acceptable aqueous or non-aqueous solvent. Aerosol formulations can be presented in single or multidose quantities in sterile form in a sealed container, which can take the form of a cartridge or refill for use with an atomising device or inhaler. Alternatively the sealed container may be a unitary dispensing device such as a single dose nasal inhaler or an aerosol dispenser fitted with a metering valve (metered dose inhaler) which is intended for disposal once the contents of the container have been exhausted.

Where the dosage form comprises an aerosol dispenser, it preferably contains a suitable propellant under pressure such as compressed air, carbon dioxide or an organic propellant such as a hydrofluorocarbon (HFC). Suitable HFC propellants include 1,1,1,2,3,3,3-heptafluoropropane and 1,1,1,2-tetrafluoroethane. The aerosol dosage forms can also take the form of a pump-atomiser. The pressurised aerosol may contain a solution or a suspension of the active compound. This may require the incorporation of additional excipients e.g. co-solvents and/or surfactants to improve the dispersion characteristics and homogeneity of suspension formulations. Solution formulations may also require the addition of co-solvents such as ethanol. Other excipient modifiers may also be incorporated to improve, for example, the stability and/or taste and/or fine particle mass characteristics (amount and/or profile) of the formulation.

For pharmaceutical compositions suitable and/or adapted for inhaled administration, the pharmaceutical composition may be a dry powder inhalable composition. Such a composition can comprise a powder base such as lactose, glucose, trehalose, mannitol or starch, the compound of formula (I) or salt or solvate thereof (preferably in particle-size-reduced form, e.g. in micronised form), and optionally a performance modifier such as L-leucine or another amino acid, cellobiose octaacetate and/or metals salts of stearic acid such as magnesium or calcium stearate. Preferably, the dry powder inhalable composition comprises a dry powder blend of lactose and the compound of formula (I) or salt thereof. The lactose is preferably lactose hydrate e.g. lactose monohydrate and/or is preferably inhalation-grade and/or fine-grade lactose. Preferably, the particle size of the lactose is defined by 90% or more (by weight or by volume) of the lactose particles being less than 1000 microns (micrometres) (e.g. 10-1000 microns e.g. 30-1000 microns) in diameter, and/or 50% or more of the lactose particles being less than 500 microns (e.g. 10-500 microns) in diameter. More preferably, the particle size of the lactose is defined by 90% or more of the lactose particles being less than 300 microns (e.g. 10-300 microns e.g. 50-300 microns) in diameter, and/or 50% or more of the lactose particles being less than 100 microns in diameter. Optionally, the particle size of the lactose is defined by 90% or more of the lactose particles being less than 100-200 microns in diameter, and/or 50% or more of the lactose particles being less than 40-70 microns in diameter. Most importantly, it is preferable that about 3 to about 30% (e.g. about 10%) (by weight or by volume) of the particles are less than 50 microns or less than 20 microns in diameter. For example, without limitation, a suitable inhalation-grade lactose is E9334 lactose (10% fines) (Borculo Domo Ingredients, Hanzeplein 25, 8017 J D Zwolle, Netherlands).

Optionally, in particular for dry powder inhalable compositions, a pharmaceutical composition for inhaled administration can be incorporated into a plurality of sealed dose containers (e.g. containing the dry powder composition) mounted longitudinally in a strip or ribbon inside a suitable inhalation device. The container is rupturable or peel-openable on demand and the dose of e.g. the dry powder composition can be administered by inhalation via the device such as the DISKUS™ device, marketed by GlaxoSmithKline. The DISKUS™ inhalation device is for example described in GB 2242134 A, and in such a device at least one container for the pharmaceutical composition in powder form (the container or containers preferably being a plurality of sealed dose containers mounted longitudinally in a strip or ribbon) is defined between two members peelably secured to one another; the device comprises: a means of defining an opening station for the said container or containers; a means for peeling the members apart at the opening station to open the container; and an outlet, communicating with the opened container, through which a user can inhale the pharmaceutical composition in powder form from the opened container.

The compounds of the invention thereof may be formulated as a fluid formulation for delivery from a fluid dispenser, for example a fluid dispenser having a dispensing nozzle or dispensing orifice through which a metered dose of the fluid formulation is dispensed upon the application of a user-applied force to a pump mechanism of the fluid dispenser. Such fluid dispensers are generally provided with a reservoir of multiple metered doses of the fluid formulation, the doses being dispensable upon sequential pump actuations. The dispensing nozzle or orifice may be configured for insertion into the nostrils of the user for spray dispensing of the fluid formulation into the nasal cavity. A fluid dispenser of the aforementioned type is described and illustrated in WO-A-2005/044354, the entire content of which is hereby incorporated herein by reference. The dispenser has a housing which houses a fluid discharge device having a compression pump mounted on a container for containing a fluid formulation. The housing has at least one finger-operable side lever which is movable inwardly with respect to the housing to cam the container upwardly in the housing to cause the pump to compress and pump a metered dose of the formulation out of a pump stem through a nasal nozzle of the housing. A particularly preferred fluid dispenser is of the general type illustrated in FIGS. 30-40 of WO-A-2005/044354.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the compositions may include other agents conventional in the art having regard to the type of formulation in question, for example those suitable for oral administration may include flavouring agents.

A therapeutically effective amount of a compound of the present invention will depend upon a number of factors including, for example, the age and weight of the animal, the precise condition requiring treatment and its severity, the nature of the formulation, and the route of administration, and will ultimately be at the discretion of the attendant physician or veterinarian. In the pharmaceutical composition, each dosage unit for oral or parenteral administration preferably contains from 0.01 to 3000 mg, more preferably 0.5 to 1000 mg, of a compound of the invention calculated as the free base. Each dosage unit for nasal or inhaled administration preferably contains from 0.001 to 50 mg, more preferably 0.01 to 5 mg, of a compound of the formula (I) or a pharmaceutically acceptable salt thereof, calculated as the free base.

The pharmaceutically acceptable compounds the invention can be administered in a daily dose (for an adult patient) of, for example, an oral or parenteral dose of 0.01 mg to 3000 mg per day or 0.5 to 1000 mg per day, or a nasal or inhaled dose of 0.001 to 50 mg per day or 0.01 to 5 mg per day, of the compound of the formula (I) or a pharmaceutically acceptable salt thereof, calculated as the free base. This amount may be given in a single dose per day or more usually in a number (such as two, three, four, five or six) of sub-doses per day such that the total daily dose is the same. An effective amount of a salt or solvate, thereof, may be determined as a proportion of the effective amount of the compound of formula (I) per se.

The compounds of the invention and may be employed alone or in combination with other therapeutic agents. Combination therapies according to the present invention thus comprise the administration of at least one compound of formula (I) or a pharmaceutically acceptable salt or solvate thereof, and the use of at least one other pharmaceutically active agent. Preferably, combination therapies according to the present invention comprise the administration of at least one compound of formula (I) or a pharmaceutically acceptable salt or solvate thereof, and at least one other pharmaceutically active agent.

The compound(s) of the invention and the other pharmaceutically active agent(s) may be administered together in a single pharmaceutical composition or separately and, when administered separately this may occur simultaneously or sequentially in any order. The amounts of the compound(s) of the invention and the other pharmaceutically active agent(s) and the relative timings of administration will be selected in order to achieve the desired combined therapeutic effect. Thus in a further aspect, there is provided a combination comprising a compound of the invention and at least one other pharmaceutically active agent.

Thus in one aspect, the compound and pharmaceutical compositions according to the invention may be used in combination with or include one or more other therapeutic agents, for example selected from anti-inflammatory agents (including a steroid), anticholinergic agents (particularly an M₁/M₂/M₃ receptor antagonist), β₂-adrenoreceptor agonists, anti-allergy agents, antiinfective agents (such as antibiotics or antivirals), or antihistamines. The invention thus provides, in a further aspect, a combination comprising a compound of formula (I) or a pharmaceutically acceptable salt, or solvate thereof together with one or more other therapeutically active agents, for example selected from an anti-inflammatory agent such as a corticosteroid or an NSAID, an anticholinergic agent, a β₂-adrenoreceptor agonist, an anti-allergy agent, an antiinfective agent (such as an antibiotic or an antiviral), or an antihistamine.

It will be appreciated that when the compound of the present invention is administered in combination with other therapeutic agents normally administered by the inhaled, intravenous, oral or intranasal route, that the resultant pharmaceutical composition may be administered by the same routes. Alternatively the individual components of the composition may be administered by different routes.

One embodiment of the invention encompasses combinations comprising one or two other therapeutic agents.

Suitable anti-inflammatory agents include corticosteroids. Anti-inflammatory corticosteroids are well known in the art. Representative examples include fluticasone propionate, beclomethasone 17-propionate ester, beclomethasone 17,21-dipropionate ester, dexamethasone or an ester thereof, mometasone or an ester thereof (e.g. mometasone furoate), ciclesonide, budesonide, flunisolide, methyl prednisolone, prednisolone, and dexamethasone. Further examples of anti-inflammatory corticosteroids are described in WO 02/12266 A1 (Glaxo Group Ltd), in particular, the compounds of Example 1 (6α,9α-difluoro-17α-[(2-furanylcarbonyl)oxy]-11β-hydroxy-16α-methyl-3-oxo-androsta-1,4-diene-17β-carbothioic acid S-fluoromethyl ester) and Example 41 (6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester), or a pharmaceutically acceptable salt thereof.

Examples of β₂-adrenoreceptor agonists include salmeterol (e.g. as racemate or a single enantiomer such as the R-enantiomer), salbutamol, formoterol, salmefamol, fenoterol or terbutaline and salts thereof, for example the xinafoate salt of salmeterol, the sulphate salt or free base of salbutamol or the fumatrate salt of formoterol. In one embodiment, the B₂-adrenoreceptor agonists are long-acting B₂-adrenoreceptor agonists, for example, those having a therapeutic effect over a 24 hour period, such as salmeterol or formoterol.

Examples of anticholinergic compounds which may be used in combination with a compound of formula (I) or a pharmaceutically acceptable salt thereof are described in WO 03/011274 A2 and WO 02/069945 A2/US 2002/0193393 A1 and US 2002/052312 A1. For example, anticholinergic agents include muscarinic receptor antagonists, in particular, compounds which are antagonists of the M₁ or M₃ receptors, dual antagonists of M₁/M₃ or M₂/M₃ receptors or par antagonists of M₁/M₂/M₃ receptors such as ipratropium bromide, oxitropium bromide or tiotropium bromide.

An anti-histamine usable in a combination of a compound of the invention can for example be methapyrilene or H1 antagonists. Examples of H1 antagonists include, without limitation, amelexanox, astemizole, azatadine, azelastine, acrivastine, brompheniramine, cetirizine, levocetirizine, efletirizine, chlorpheniramine, clemastine, cyclizine, carebastine, cyproheptadine, carbinoxamine, descarboethoxyloratadine, doxylamine, dimethindene, ebastine, epinastine, efletirizine, fexofenadine, hydroxyzine, ketotifen, loratadine, levocabastine, mizolastine, mequitazine, mianserin, noberastine, meclizine, norastemizole, olopatadine, picumast, pyrilamine, promethazine, terfenadine, tripelennamine, temelastine, trimeprazine and triprolidine, particularly cetirizine, levocetirizine, efletirizine and fexofenadine. In a further embodiment the invention provides a combination comprising a compound of the invention together with an H3 antagonist (and/or inverse agonist). Examples of H3 antagonists include, for example, those compounds disclosed in WO2004/035556 and in WO2006/045416.

Other suitable combinations include, for example combinations comprising a compound of the invention together with other anti-inflammatory agents such as an anti-flammatory corticosteroid; or a non-steroidal anti-inflammatory drug (NSAID) such as leukotriene antagonist (e.g. montelukast), an iNOS inhibitor, a tryptase inhibitor, IKK2 inhibitors, A2a agonist, Syk inhibitors, an elastase inhibitor, a beta-2 integrin antagonist, an adenosine a2a agonist, a chemokine antagonist such as a CCR3 antagonist, or a 5-lipoxygenase inhibitor, or an antinfective agent (e.g. an antibiotic or an antiviral).

The combinations referred to above may conveniently be presented for use in the form of a pharmaceutical composition and thus pharmaceutical compositions comprising a combination as defined above together with a pharmaceutically acceptable diluent or carrier represent a further aspect of the invention. These combinations are of particular interest in respiratory diseases.

Thus in one aspect, the present invention also provides for so-called “triple combination” therapy, comprising a compound of the invention together with β2-adrenoreceptor agonist and an anti-inflammatory corticosteroid. Preferably this combination is for treatment and/or prophylaxis of asthma, COPD or allergic rhinitis. The 2-adrenoreceptor agonist and/or the anti-inflammatory corticosteroid can be as described above and/or as described in WO 03/030939 A1. A representative example of such a “triple” combination comprises a compound of the invention, salmeterol or a pharmaceutically acceptable salt (e.g. salmeterol xinafoate) and fluticasone propionate.

Rheumatoid arthritis (RA) is a further inflammatory disease where combination therapy may be contemplated. Thus in a further aspect, the present invention provides a compound of the invention in combination with a further therapeutic agent useful in the treatment of rheumatoid arthritis, said combination being useful for the treatment of rheumatoid arthritis.

The compound and pharmaceutical compositions according to the invention may be used in combination with or include one or more other therapeutic agents, for example selected from NSAIDS, corticosteroids, COX-2 inhibitors, cytokine inhibitors, anti-TNF agents, inhibitors of oncostatin M, anti-malarials, immunosuppressive and cytostatics.

Two classes of mediation are contemplated for the treatment of RA, these may be classified as “fast acting” and “slow acting” or “second line” drugs (also referred to as Disease Modifying Antirheumatic Drugs or DMARDS). The first line drugs such as typical NSAIDs (e.g. aspirin, ibuprofen, naproxen, etodolac), corticosteroids (e.g. prednisone). Second line drugs include COX-2 inhibitors and anti-TNF agents. Examples of COX-2 inhibitors are celecoxib (Celebrex), etoricoxib and rofecoxib (Vioxx).

Anti-TNF agents include infliximab (Remicade), etanercept (Enbrel) and adalimum (Humira). Other “biological” treatments include anakinra (Kineret), Rituximab, Lymphostat-B, BAFF/APRIL inhibitors and CTLA-4-Ig or mimetics thereof. Other cytokine inhibitors include lefluonomide (Arava). Further second line drugs include gold preparations (Auranofin (Ridaura tablets) or Aurothiomalate (Myocrisin injection)), medicines used for malaria: (Hydroxychloroquine (Plaquenil)), medicines that suppress the immune system (Azathioprine (Imuran, Thioprine), methotrexate (Methoblastin, Ledertrexate, Emethexate), cyclosporine (Sandimmun, Neoral)), Cyclophosphamide (Cycloblastin), Cytoxan, Endoxan), D-Penicillamine (D-Penamine), Sulphasalazine (Salazopyrin), nonsteroidal anti inflammatory drugs (including aspirin and ibrufen).

It will be clear to a person skilled in the art that, where appropriate, the other therapeutic ingredient(s) may be used in the form of salts, for example as alkali metal or amine salts or as acid addition salts, or prodrugs, or as esters, for example lower alkyl esters, or as solvates, for example hydrates, to optimise the activity and/or stability and/or physical characteristics, such as solubility, of the therapeutic ingredient. It will be clear also that, where appropriate, the therapeutic ingredients may be used in optically pure form.

The combinations referred to above may conveniently be presented for use in the form of a pharmaceutical composition and thus pharmaceutical compositions comprising a combination as defined above together with a pharmaceutically acceptable diluent or carrier represent a further aspect of the invention.

The individual compounds of such combinations may be administered either sequentially or simultaneously in separate or combined pharmaceutical compositions. Preferably, the individual compounds will be administered simultaneously in a combined pharmaceutical composition. Appropriate doses of known therapeutic agents will be readily appreciated by those skilled in the art.

Biological Test Methods

PDE3, PDE4B, PDE4D, PDE5 Primary Assay Methods

The activity of the compounds may be measured as described below. Preferred compounds of the invention are selective PDE4 inhibitors, i.e. they inhibit PDE4 (e.g. PDE4B and/or PDE4D) more strongly than they inhibit other PDE's such as PDE3 and/or PDE5.

PDE Enzyme Sources and Literature References

Human recombinant PDE4B, in particular the 2B splice variant thereof (HSPDE4B2B), is disclosed in WO 94/20079 and also in M. M. McLaughlin et al., “A low Km, rolipram-sensitive, cAMP-specific phosphodiesterase from human brain: cloning and expression of cDNA, biochemical characterisation of recombinant protein, and tissue distribution of mRNA”, J. Biol. Chem., 1993, 268, 6470-6476. For example, in Example 1 of WO 94/20079, human recombinant PDE4B is described as being expressed in the PDE-deficient yeast Saccharomyces cerevisiae strain GL62, e.g. after induction by addition of 150 uM CuSO₄, and 100,000×g supernatant fractions of yeast cell lysates are described for use in the harvesting of PDE4B enzyme.

Human recombinant PDE4D (HSPDE4D3A) is disclosed in P. A. Baecker et al., “Isolation of a cDNA encoding a human rolipram-sensitive cyclic AMP phoshodiesterase (PDE IV_{D})”, Gene, 1994, 138, 253-256.

Human recombinant PDE5 is disclosed in K. Loughney et al., “Isolation and characterisation of cDNAs encoding PDE5A, a human cGMP-binding, cGMP-specific 3′,5′-cyclic nucleotide phosphodiesterase”, Gene, 1998, 216, 139-147.

PDE3 may be purified from bovine aorta as described by H. Coste and P. Grondin, “Characterisation of a novel potent and specific inhibitor of type V phosphodiesterase”, Biochem. Pharmacol., 1995, 50, 1577-1585.

PDE6 may be purified from bovine retina as described by: P. Catty and P. Deterre, “Activation and solubilization of the retinal cGMP-specific phosphodiesterase by limited proteolysis”, Eur. J. Biochem., 1991, 199, 263-269; A. Tar et al. “Purification of bovine retinal cGMP phosphodiesterase”, Methods in Enzymology, 1994, 238, 3-12; and/or D. Srivastava et al. “Effects of magnesium on cyclic GMP hydrolysis by the bovine retinal rod cyclic GMP phosphodiesterase”, Biochem. J., 1995, 308, 653-658.

Inhibition of PDE Activity: Fluorescence Polarisation (FP) Assay

The ability of compounds to inhibit PDE catalytic activity was determined by IMAP Fluorescence Polarisation (FP) assay (Molecular Devices Ltd code: R8062) in 384-well format. Test compounds (small volume, e.g. 0.5 μl, of solution in DMSO) were preincubated at ambient temperature in black 384-well microtitre plates (supplier: NUNC, code 262260) with PDE enzyme in 10 mM Tris-HCl buffer pH 7.2, 10 mM MgCl₂, 0.1% (w/v) bovine serum albumin, 0.05% NaN₃ for 10-30 minutes. The enzyme level was set so that reaction was linear throughout the incubation.

For the PDE3, PDE4B and PDE4D assays Fluorescein adenosine 3′,5′-cyclic phosphate (Molecular Devices Ltd code: R7091) was added to give ˜40 nM final concentration. For the PDE5 and PDE6 assays Fluorescein guanosine 3′,5′-cyclic phosphate (Molecular Devices Ltd code: R7090) was added to give ˜40 nM final concentration. Plates were mixed on an orbital shaker for 10 seconds and incubated at ambient temperature for 40 minutes. IMAP binding reagent (Molecular Devices Ltd code: R7207) was added (60 μl of a 1 in 400 dilution in binding buffer of the kit stock solution) to terminate the assay. Plates were allowed to stand at ambient temperature for 1 hour. The FP ratio of parallel to perpendicular light was measured using an Analyst™ plate reader (from Molecular Devices Ltd). For inhibition curves, 11 concentrations (0.5 nM-30 μM) of each compound were assayed; more potent compounds were assayed over lower concentration ranges (assay concentrations were generally between 30 μM and 50 fM). Curves were analysed using ActivityBase and XLfit (ID Business Solutions Limited). Results were expressed as pIC₅₀ values.

Inhibition of PDE3, PDE4B, PDE4D, PDE5 or PDE6 Activity: Radioactive Scintillation Proximity Assay (SPA)

The ability of compounds to inhibit catalytic activity at PDE4B or 4D (human recombinant), PDE3 (from bovine aorta), PDE5 (human recombinant) or PDE 6 (from bovine retina) was determined by Scintillation Proximity Assay (SPA) in 96-well format. Test compounds (preferably as a solution in DMSO, e.g. 2 microlitre (μl) volume) were preincubated at ambient temperature in Wallac Isoplates (code 1450-514) with PDE enzyme in 50 mM Tris-HCl buffer pH 7.5, 8.3 mM MgCl₂, 1.7 mM EGTA, 0.05% (w/v) bovine serum albumin for 10-30 minutes. The enzyme concentration was adjusted so that no more than 20% hydrolysis of the substrate occurred in control wells without compound, during the incubation. For the PDE3, PDE4B and PDE4D assays [5′,8-³H]adenosine 3′,5′-cyclic phosphate (Amersham Pharmacia Biotech, code TRK.559 or Amersham Biosciences UK Ltd, Pollards Wood, Chalfont St Giles, Buckinghamshire HP8 4SP, UK) was added to give 0.05 μCi per well and ˜10 nM final concentration. For the PDE5 and PDE6 assays [8-³H]guanosine 3′,5′-cyclic phosphate (Amersham Pharmacia Biotech , code TRK.392) was added to give 0.05 μCi per well and ˜36 nM final concentration. Plates e.g. containing approx. 100 μl volume of assay mixture were mixed on an orbital shaker for 5 minutes and incubated at ambient temperature for 1 hour. Phosphodiesterase SPA beads (Amersham Pharmacia Biotech, code RPNQ 0150) suspended in buffer were added (˜1 mg per well) to terminate the assay. Plates were sealed and shaken and allowed to stand at ambient temperature for 35 minutes to 1 hour to allow the beads to settle. Bound radioactive product was measured using a WALLAC TRILUX 1450 Microbeta scintillation counter. For inhibition curves, 10 concentrations (e.g. 1.5 nM-30 μM) of each compound were assayed; more potent compounds were assayed over lower concentration ranges (assay concentrations were generally between 30 μM and 50 fM). Curves were analysed using ActivityBase and XLfit (ID Business Solutions Limited, 2 Ocean Court, Surrey Research Park, Guildford, Surrey GU2 7QB, United Kingdom). Results were expressed as pIC₅₀ values.

The compound of Example 1 inhibits the catalytic activity at the PDE4B (human recombinant) enzyme with pIC₅₀ of about 10 (FP assay described above or similar)

Using a mixture of the SPA and FP assays as described above (or similar), the compounds of the Examples exhibited PIC₅₀ of 7.0-10.5.

Emesis: Many known PDE4 inhibitors cause emesis and/or nausea to greater or lesser extents (e.g. see Z. Huang et al., Current Opinion in Chemical Biology, 2001, 5, 432-438, see especially pages 433-434 and refs cited therein). Therefore, it would be preferable but not essential that a PDE4 inhibitory compound of the invention causes only limited or manageable emetic side-effects. Emetic side-effects can for example be measured by the emetogenic potential of the compound when administered to ferrets; for example one can measure the time to onset, extent, frequency and/or duration of vomiting and/or writhing in ferrets after oral or parenteral administration of the compound. See for example A. Robichaud et al., “Emesis induced by inhibitors of PDE IV in the ferret” Neuropharmacology, 1999, 38, 289-297, erratum Neuropharmacology, 2001, 40, 465-465.

All publications, including but not limited to patents and patent applications, cited in this specification are herein incorporated by reference as if each individual publication were specifically and individually indicated to be incorporated by reference herein as though fully set forth.

General Experimental Details

### EXAMPLES

Abbreviations used herein:
HPLC high performance liquid chromatography
LC/MS liquid chromatography/mass spectroscopy
SPE solid phase extraction column. Unless otherwise specified the solid phase will be silica gel. Aminopropyl SPE refers to a silica SPE column with aminopropyl residues immobilised on the solid phase (eg. IST Isolute™ columns). It is thought that compounds isolated by SPE are free bases.
Oxone this refers to Oxone®, which is potassium peroxymonosulphate, 2KHSO₅.KHSO₄.K₂SO₄
g grams);
mg milligrams);
μl microlitres;
l litres
μg micrograms
M molar
nM millimolar
mol moles;
EtOH ethernol;
Mmol millimoles;
Rt room temperature;
Min minutes;
H hours;
Mp melting point;
MeOH methanol;
THF tetrahydrofuran;
DMSO dimethylsulfoxide;
AcOEt ethyl acetate;
DMF N,N-dimethylformamide;
BOC tert-butyloxycarbonyl;
Ac acetyl;
Pd2(dba)3 tris (dibenzylideneacetone) dipalladium (O)
DPE Phos oxydi-2,-1-phenylene)bis(diphenylphosphine)
Rₜ Retention time

General Experimental Details

LC/MS (Liquid Chromatography/Mass Spectroscopy)

Waters ZQ mass spectrometer operating in positive ion electrospray mode, mass range

100-1000 amu. UV wavelength: 215-330 nm

Column: 3.3 cm×4.6 mm ID, 3 μm ABZ+PLUS

Flow Rate: 3 ml/min

Injection Volume: 5 μl

Solvent A: 95% acetonitrile+0.05% of a 1% v/v solution of formic acid in water

Solvent B: 0.1% v/v solution of formic acid in 10 mM aqueous ammonium acetate

Gradient: Mixtures of Solvent A and Solvent B are used according to the following gradient profiles (expressed as % Solvent A in the mixture): 0% A/0.7 min, 0-100% A/3.5 min, 100% A/1.1 min, 100-0% A/0.2 min

Mass Directed Automated Preparative HPLC Column, Conditions and Eluent

The preparative column used was typically a Supelcosil ABZplus (10 cm×2.12 cm internal diameter; particle size 5 μm)

UV detection wavelength: 200-320 nm

Flow rate: 20 ml/min

Injection Volume: 0.5 ml

Solvent A: 0.1% v/v solution of formic acid in water

Solvent B: 95% acetonitrile+5% of a 1% v/v solution of formic acid in water

Gradient systems: mixtures of Solvent A and Solvent B are used according to a choice of 5 generic gradient profiles (expressed as % Solvent B in the mixture), ranging from a start of 0 to 50% Solvent B, with all finishing at 100% Solvent B to ensure total elution. It is thought that compounds isolated by this method are free bases.

‘Hydrophobic Frit’

This refers to a Whatman PTFE filter medium (frit), pore size 5.0 μm, housed in a polypropylene tube.

Evaporation of Product Fractions after Purification

Reference to SPE and preparative HPLC purification includes evaporation of the product containing fractions to dryness by an appropriate method.

Aqueous Ammonia Solutions

‘880 Ammonia’ or ‘0.880 ammonia’ refers to concentrated aqueous ammonia (specific gravity 0.880).

Celite

References to celite generally refer to the filter agent Celite® 545 (e.g. available from Aldrich).

Florisil

References to florisil generally refer to the magnesium silicate Florisil®, for example 60-100 mesh (available from Aldrich).

Smopex-111®

References to Smopex-111® refer to the thiol group bearing metal scavenging fibre of that name (available from Johnson Matthey).

Intermediates and Examples

When the name of a commercial supplier is given after the name of a compound or a reagent, for instance “compound X (Aldrich)” or “compound X/Aldrich”, this means that compound X is obtainable from a commercial supplier, such as the commercial supplier named.

Similarly, when a literature or a patent reference is given after the name of a compound, for instance compound Y (EP 0 123 456), this means that the preparation of the compound is described in the named reference.

The names of the Examples have been obtained using a compound naming programme which matches structure to name (e.g. ACD/Name Batch v 9.0).

### Intermediate 1. Diethyl [(4-iodo-2-methylphenyl)hydrazono]propanedioate

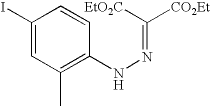

To 4-iodo-2-methylaniline (23.3 g) (available from Fluorochem) in ice (60 g) and concentrated hydrochloric acid (30 ml) was added a solution of sodium nitrite (7.05 g) in water (18 ml) slowly with stirring over 20 min, keeping the temperature below 5° C. The mixture was allowed to stand at 0-5° C. for 2 h, then added slowly at 0-5° C. over 30 min to a mixture of diethyl malonate (17 ml), ethanol (300 ml) and water (50 ml) containing sodium acetate (18.8 g). The mixture was stirred for 2 h at 0-5° C. then for 2 h at 21° C. The mixture was concentrated in vacuo to a volume of about 200 ml, and the product was collected by filtration, washed with water (200 ml) and dried under vacuum to give the title compound as a brown solid (28.9 g).

LC/MS Rₜ 3.85 min m/z 405 [MH⁺].

### Intermediate 2. [(4-Iodo-2-methylphenyl)hydrazono]propanedioic acid

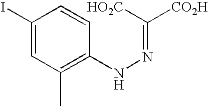

To a solution of diethyl [(4-iodo-2-methylphenyl)hydrazono]propanedioate (5.02 g) (for example, as prepared for Intermediate 1) in ethanol (60 ml) was added 2M sodium hydroxide (6.2 ml) and the mixture was heated to 50° C. for 10 min (with stirring with a spatula). A further portion of 2M sodium hydroxide (12.4 ml) and water (10 ml) were added and heating was continued at 80° C. for 0.5 h. Water (50 ml) was added to the mixture followed by concentrated hydrochloric acid (10 ml, added slowly). The precipitate was collected by filtration and dried in vacuo to give the title compound as a brown solid (3.56 g).

LC/MS Rₜ 4.46 min m/z 347 [MH⁻].

### Intermediate 3. [(4-Iodo-2-methylphenyl)hydrazono]propanedioyl dichloride

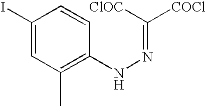

To [(4-iodo-2-methylphenyl)hydrazono]propanedioic acid (20.1 g) (for example, as prepared for Intermediate 2) in chloroform (150 ml) was added thionyl chloride (25 ml) and the mixture heated under reflux under nitrogen for 6 h. The mixture was cooled to room temperature over 16 h, then heated under reflux for 3 h. Further thionyl chloride (15 ml) was added and the mixture heated under reflux for 4 h under nitrogen and then left to cool to room temperature over 16 h. The solvent was removed in vacuo, azeotroping with toluene to give the title compound as a brown solid (22.0 g).

LC/MS (sample quenched with methanol) Rₜ 3.53 min m/z 377 [MH⁺]

### Intermediate 4. Butyl 6-iodo-8-methyl-4-oxo-1,4-dihydro-3-cinnolinecarboxylate

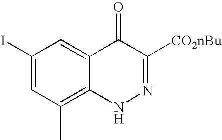

To [(4-iodo-2-methylphenyl)hydrazono]propanedioyl dichloride (22.0 g) (for example, as prepared for Intermediate 3) in 1,2-dichloroethane (175 ml) was added titanium tetrachloride (16.3 g) and the mixture heated under nitrogen under reflux for 16 h. The mixture was cooled to room temperature and then cooled further in an ice/water bath. n-Butanol (50 ml) was added and the mixture allowed to warm to room temperature. The mixture was partitioned between water (500 ml) and ethyl acetate (3×400 ml). The combined organic layers were dried over magnesium sulphate and the solvent removed in vacuo to give the title compound (21.6 g).

LC/MS Rₜ 3.21 min m/z 387 [MH⁺]

### Intermediate 5. 6-Iodo-8-methyl-4-oxo-1,4-dihydro-3-cinnolinecarboxylic acid

Method A

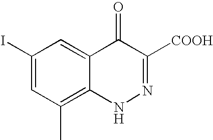

[(4-Iodo-2-methylphenyl)hydrazono]propanedioic acid (5.0 g) (for example, as prepared for Intermediate 2) in chloroform (215 ml) was treated with thionyl chloride (8 ml) and the mixture heated under reflux for 17 h. The chloroform was removed in vacuo and the residue co-evaporated with chloroform (100 ml). The residue was dissolved in nitrobenzene (200 ml), titanium IV chloride (6 ml) added, and the mixture was heated at 100° C. for 3 h. The cooled mixture was poured onto 2M sodium hydroxide solution (70 ml) and ice (100 g) and the solid removed by filtration. The solid was suspended in hot water (150 ml) and filtered, re-suspended in hot 0.4M sodium hydroxide solution (200 ml) and filtered again. The aqueous extracts were combined and concentrated in vacuo to approximately 500 ml, then acidified to pH 3 using concentrated hydrochloric acid. The mixture was allowed to stand overnight and the product collected by filtration, washed with water (100 ml) and suction dried to give the title compound (2.14 g).

LC/MS Rₜ 3.17 min m/z 331 [MH⁺].

The mother liquors were concentrated to approximately 150 ml, filtered and washed with water (20 ml) then suction dried to give a further quantity of the title compound (0.36 g).

LC/MS Rₜ 3.21 min m/z 331 [MH⁺].

### Intermediate 5. 6-Iodo-8-methyl-4-oxo-1,4-dihydro-3-cinnolinecarboxylic acid

Method B

Butyl 6-iodo-8-methyl-4-oxo-1,4-dihydro-3-cinnolinecarboxylate (21.6 g) (for example, as prepared for Intermediate 4) was placed in ethanol (200 ml) and 2M sodium hydroxide (100 ml) added. The resultant mixture was stirred at 80° C. for 90 minutes. The mixture was cooled to room temperature and acidified to pH 1 by the addition of 2M hydrochloric acid. The resultant precipitate was collected by filtration, the filter cake washed with water and dried in a vacuum oven at 50° C. for 16 h to give the title compound as a fawn solid (18.5 g).

LC/MS Rₜ 3.12 min m/z 331 [MH⁺]

### Intermediate 5. 6-Iodo-8-methyl-4-oxo-1,4-dihydro-3-cinnolinecarboxylic acid

Method C

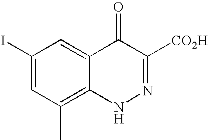

To a stirred suspension of ethyl 6-iodo-8-methyl-4-oxo-1,4-dihydro-3-cinnolinecarboxylate (23.9 g, 0.067 mol) (for example, as prepared for Intermediate 57) in methanol (300 ml) was added 2M sodium hydroxide (150 ml). The mixture was heated at reflux with overhead mechanical stirring for 2 h causing it to become much thicker, then cooled to room temperature and poured into 1M hydrochloric acid (800 ml) with stirring. The precipitate was filtered, washed with water and dried in vacuo at 50° C. to obtain the title compound as a cream-coloured solid (21.7 g, 98%).

LC/MS Rₜ 3.07 min m/z 331 [MH⁺].

### Intermediate 6.4-Chloro-6-iodo-8-methyl-3-cinnolinecarboxamide

Method A

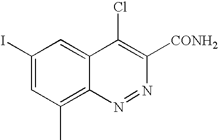

A suspension of 6-iodo-8-methyl-4-oxo-1,4-dihydro-3-cinnolinecarboxylic acid (2.1 g) (for example, as prepared for Intermediate 5) in thionyl chloride (15 ml) was heated at 80° C. for 2 h. The mixture was concentrated in vacuo and the residue azeotroped with toluene (5 ml). The residue was dissolved in 1,4-dioxane (10 ml) and added dropwise to 880 ammonia (30 ml) with vigorous stirring to give a suspension. The solid was removed by filtration to give the title compound as a brown solid (1.77 g).

LC/MS Rₜ 3.04 min m/z 348 [MH⁺].

### Intermediate 6. 4-Chloro-6-iodo-8-methyl-3-cinnolinecarboxamide

Method B

6-Iodo-8-methyl-4-oxo-1,4-dihydro-3-cinnolinecarboxylic acid (10.55 g) (for example, as prepared for Intermediate 5) was heated at 90° C. (with phosphorous oxychloride (10 ml) with stirring under nitrogen for 2 h. Excess phosphorous oxychloride was evaporated in vacuo, and the residue cooled in an ice/water bath. 880 ammonia (20 ml) was cautiously added, and the resulting solid agitated with a spatula before stirring at 0° C.—room temperature for 2 h. The solid was filtered off, washed with water, and dried to give the title compound as a grey solid (444 mg).

LC/MS Rₜ 2.86 min m/z 348 [MH⁺].

### Intermediate 6. 4-Chloro-6-iodo-8-methyl-3-cinnolinecarboxamide

Method C

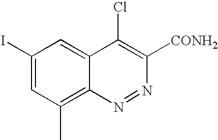

A stirred suspension of 6-iodo-8-methyl-4-oxo-1,4-dihydro-3-cinnolinecarboxylic acid (21.5 g, 0.065 mol) (for example, as prepared for Intermediate 5) in phosphorous oxychloride (200 ml) was heated to reflux. (As the mixture heated up it became much thicker). After heating at reflux for 2 h the resulting dark green solution was cooled to room temperature then evaporated in vacuo. The residue was azeotroped with toluene (×2) then treated with 1,4-dioxane (40 ml; not completely soluble). The solution/suspension was added slowly to ice-cooled 880 ammonia (400 ml) with stirring. The precipitate was filtered, washed with water and dried in vacuo at 50° C. to obtain the title compound as a dark brown solid (21.2 g, 94%).

LC/MS Rₜ 2.85 min m/z 348 [MH⁺].

### Intermediate 7. 4-[(3-Cyanophenyl)amino]-6-iodo-8-methyl-3-cinnolinecarboxamide

Method A

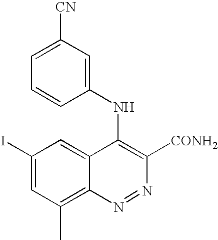

A suspension of 4-chloro-6-iodo-8-methyl-3-cinnolinecarboxamide (0.7 g) (for example, as prepared for Intermediate 6) in acetonitrile (50 ml) was treated with 3-aminobenzonitrile (0.25 g) and the mixture was heated at 90° C. for 18 h. The precipitate was removed by filtration and the filtrate concentrated in vacuo to give an orange solid (0.88 g). This was triturated with acetonitrile (10 ml) and the solid collected by filtration to give the title compound as an orange solid (0.68 g).

LC/MS Rₜ 3.21 min m/z 430 [MH⁺]; minor impurity Rt 3.09 min, m/z 338

### Intermediate 7. 4-[(3-Cyanophenyl)amino]-6-iodo-8-methyl-3-cinnolinecarboxamide

Method B

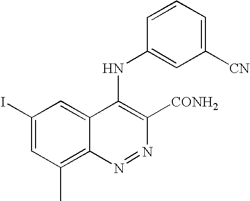

A stirred suspension of 4-chloro-6-iodo-8-methyl-3-cinnolinecarboxamide (12.1 g, 0.035 mol) (for example, as prepared for Intermediate 6) and 3-aminobenzonitrile (4.13 g, 0.035 mol) in acetonitrile (250 ml) was heated to reflux for 1 h, during which time the reaction mixture became much thicker. Analysis showed that the reaction was incomplete, and did not change after a further 1 h at reflux. Additional 3-aminobenzonitrile (0.83 g, 0.007 mol) was added and refluxing continued for 1 h. Another portion of 3-aminobenzonitrile (0.83 g, 0.007 mol) was added and the mixture was heated at reflux for a further 1.5 h. Analysis (LC-MS) showed complete reaction.

The mixture was cooled to room temperature, filtered under reduced pressure, and the filtered solid washed with acetonitrile and dried in vacuo at 45° C. to give a yellow solid (16.5 g) which contained ca. 14% by area of 3-aminobenzonitrile. The solid was suspended in acetonitrile (200 ml) and stirred at room temperature for 1.5 h, filtered and analysed by HPLC. The product still contained 3-aminobenzonitrile, which indicated it was present as its HCl salt [the aniline is freely soluble in acetonitrile]. The crude product was suspended in water (200 ml) and stirred at room temperature for 1 h, filtered and the residue washed with water. The damp cake was dried in vacuo at 50° C. to give the title compound as a pale beige solid (13.9 g, 92%).

LC/MS Rₜ 3.25 min m/z 430 [MH⁺].

### Intermediate 7. 4-[(3-Cyanophenyl)amino]-6-iodo-8-methyl-3-cinnolinecarboxamide

Method C

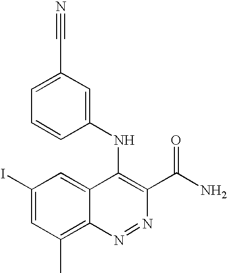

To 4-chloro-6-iodo-8-methyl-3-cinnolinecarboxamide (120 g) (for example, as prepared for Intermediate 6) in acetonitrile (2.0 l) was added 3-aminobenzonitrile (40.7 g) and the suspension heated to reflux. Viscosity increased for the first two hours then began to decrease. The reaction was followed by hplc and after five and a half hours only 2% starting cinnoline remained. The reaction was cooled and filtered under vacuum and the solid dried under high vacuum at 40° C. overnight. This gave a dark olive-yellow solid, (152 g), 96% pure by hpic.

This solid was added to well-stirred (mechanical stirrer) saturated aqueous sodium bicarbonate (1.5 l) and effervescence observed. After a 17 minute addition the suspension was stirred for a further 20 minutes until no more gas evolution was observed and the colour had become pale brown. The solid was collected by vacuum filtration and washed well with water (2 l), then dried under high vacuum at 45° C. to give the title compound as a brown solid (130 g).

LC/MS Rₜ 3.28 min m/z 430 [MH⁺].

### Intermediate 8. 4-[(3-Cyanophenyl)amino]-8-methyl-6-(methylthio)-3-cinnolinecarboxamide

Method A.

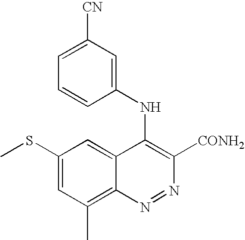

To a solution of 4-[(3-cyanophenyl)amino]-6-iodo-8-methyl-3-cinnolinecarboxamide (0.67 g) (for example, as prepared for Intermediate 7) in dry N-N′-dimethylformamide (20 ml) was added sodium thiomethoxide (0.218 g), sodium tert-butoxide (0.15 g), tris(dibenzylideneacetone)dipalladium (0) (0.143 g) and (oxydi-2,1-phenylene)bis(diphenylphosphine) (0.084 g). The mixture was heated at 100° C. for 3 h then cooled and concentrated in vacuo. The residue was partitioned between chloroform (50 ml) and water (30 ml), and the layers separated by hydrophobic frit. The organic layer was concentrated to approximately 20 ml, placed on an SPE column (100 g) and eluted with a gradient of cyclohexane and ethyl acetate to give the title compound as a yellow solid (0.51 g).

LC/MS Rₜ 3.22 min m/z 350 [MH⁺]; minor impurity Rt 3.26 min, m/z 338

### Intermediate 8. 4-[(3-Cyanophenyl)amino]-8-methyl-6-(methylthio)-3-cinnolinecarboxamide

Method B.

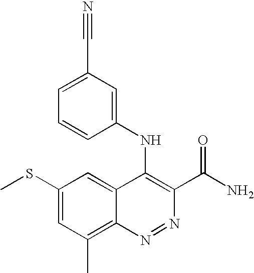

To the 4-[(3-cyanophenyl)amino]-6-iodo-8-methyl-3-cinnolinecarboxamide (66.9 g) (for example, as prepared for Intermediate 7) in nitrogen-purged dry DMF (1.5 l) was added sodium thiomethoxide (21.8 g) followed by tris(dibenzylideneacetone)-dipalladium (0) (7.1 g) and (oxydi-2,1-phenylene)bis(diphenylphosphine) (4.2 g). The mixture was heated to 100° C. under nitrogen, covered in foil to exclude as much light as possible. After two and a half hours the reaction was cooled and the solvent removed under high vacuum. The resulting red-brown oil was partitioned between dichloromethane and water and the aqueous phase extracted with more dichloromethane. The combined organic layers were washed with water and brine, dried (MgSO₄) and evaporated down to about 200 ml, whereupon an equal volume of diethyl ether was added to the slurry and the solid collected by vacuum filtration to give the title compound as a yellow solid (49.8 g, 91%), 93% pure by hplc.

LC/MS Rₜ 3.13 min m/z 350 [MH⁺].

### Intermediate 9. 4-[(1-Ethyl-1H-pyrazol-5-yl)amino]-6-iodo-8-methyl-3-cinnolinecarboxamide

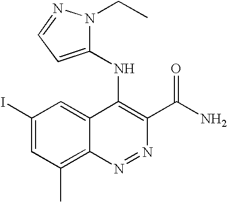

To a suspension of 4-chloro-6-iodo-8-methyl-3-cinnolinecarboxamide (1 g) (for example, as prepared for Intermediate 6) in acetonitrile (50 ml) was added 1-ethyl-1H-pyrazol-5-amine (available from Aldrich; 0.32 g) and pyridine hydrochloride (0.33 g). The mixture was heated at 80° C. for 48 h, cooled and filtered, washing with acetonitrile (10 ml) and ether (10 ml) to give a brown solid 1.06 g) which was triturated with methanol (20 ml) and filtered to give the title compound as a pale brown solid (0.63 g).

LC/MS Rt 2.99 min m/z 423 [MH⁺]

### Intermediate 10. 6-[(1,1-Dimethylethyl)thio]-4-[(1-ethyl-1H-pyrazol-5-yl)amino]-8-methyl-3-cinnolinecarboxamide

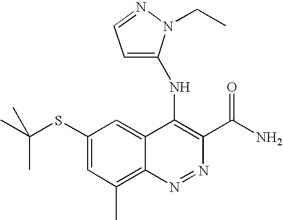

A suspension of 4-[(1-ethyl-1H-pyrazol-5-yl)amino]-6-iodo-8-methyl-3-cinnolinecarboxamide (100 mg) (for example, as prepared for Intermediate 9) in dry DMF (1 ml) was treated with tributyl[(1,1-dimethylethyl)thio]stannane (83 mg) (for example as prepared for Intermediate 60) and tetrakis(triphenylphosphine)palladium(0) (25 mg). The mixture was stirred and heated by microwave irradiation at 130° C. for 10 min and blown to dryness under a stream of nitrogen to give a dark yellow solid. The solid was triturated with 1:1 cyclohexane/ethylacetate (2 ml) and the solid collected by filtration to give the title compound as a dark yellow solid (52 mg).

LC/MS Rt 3.23 min m/z 385 [MH⁺]

### Intermediate 11. 4-[(1-Ethyl-1H-pyrazol-5-yl)amino]-6-(ethylthio)-8-methyl-3-cinnolinecarboxamide

Method A

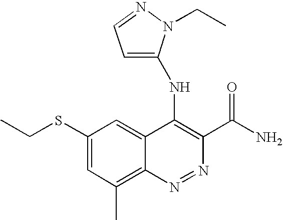

A suspension of 4-[(1-ethyl-1H-pyrazol-5-yl)amino]-6-iodo-8-methyl-3-cinnolinecarboxamide (100 mg) (for example, as prepared for Intermediate 9) in dry DMF (1 ml) was treated with tributyl(ethylthio)stannane 77 mg (for example, as prepared for Intermediate 58) and tetrakis(triphenylphosphine)palladium(0) (25 mg). The mixture was stirred and heated by microwave irradiation at 130° C. for 10 min. The mixture was poured onto an aminopropyl SPE cartridge (10 ml) and eluted with methanol. The fractions were evaporated to give a yellow solid which was triturated with 1:1 cyclohexane/ethylacetate and the solid collected by filtration to give the title compound as a pale yellow solid (19 mg).

LC/MS Rt 3.01 min m/z 357 [MH⁺]

### Intermediate 11. 4-[(1-Ethyl-1H-pyrazol-5-yl)amino]-6-(ethylthio)-8-methyl-3-cinnolinecarboxamide

Method B

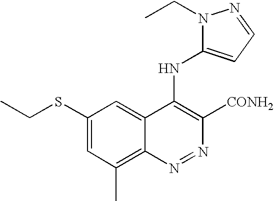

To 4-chloro-6-(ethylthio)-8-methyl-3-cinnolinecarboxamide (1.1 g) (for example, as prepared for Intermediate 13) in acetonitrile (25 ml) was added 1-ethyl-1H-pyrazol-5-amine (available from Aldrich; 0.655 g) and the mixture heated to reflux for 18 h. The mixture was diluted with acetonitrile (100 ml), pyridine hydrochloride (0.452 g) was added, and heating under reflux was continued over the weekend. The mixture was evaporated to a small volume, filtered and the residue washed with a small volume of acetonitrile then dried in vacuo. The crude product was stirred vigorously in ethyl acetate and saturated sodium bicarbonate solution. The organic layer was washed with water, dried (MgSO₄), and evaporated to give the title compound (0.67 g).

LC/MS Rₜ 3.04 min m/z 357 [MH⁺].

### Intermediate 12. 4-Chloro-8-methyl-6-(methylthio)-3-cinnolinecarboxamide

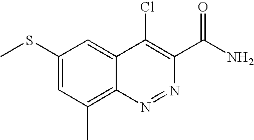

To 4-chloro-6-iodo-8-methyl-3-cinnolinecarboxamide (100 mg) (for example, as prepared for Intermediate 6) in N,N′-dimethylformamide (1 ml) was added tributyl(methylthio)stannane (Intermediate 44: 126 mg) and tetrakis(triphenylphosphine)palladium(0) (16 mg) and the mixture heated at 60° C. for 18 hours, then cooled to room temperature. The mixture was filtered through Celite® filter agent and the solvent removed in vacuo. The residue was suspended in cyclohexane:ethyl acetate (1:1) (8 ml) and the resultant precipitate collected by filtration, dried in a vacuum oven at 40° C. overnight to give the title compound as a dark brown solid (55 mg).

LC/MS Rₜ 2.70 min m/z 268 [MH⁺].

### Intermediate 13. 4-Chloro-6-(ethylthio)-8-methyl-3-cinnolinecarboxamide

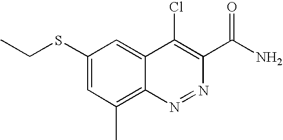

A suspension of 4-chloro-6-iodo-8-methyl-3-cinnolinecarboxamide (0.5 g) (for example, as prepared for Intermediate 6) in dry N,N-dimethylformamide (5 ml) was treated with tributyl(ethanethio)stannane (0.61 g) and tetrakis(triphenylphosphine) palladium(0) (0.166 g). The mixture was heated to 130° C. under microwave irradiation for 10 min, cooled and water (5 ml) added. The solid was collected by filtration and triturated with 1:1 ethyl acetate/cyclohexane (5 ml) and dried to give the title compound as a pale brown solid (280 mg).

LC/MS Rt 2.95 min m/z 282 [MH⁺]

### Intermediate 14. 4-Chloro-8-methyl-6-(methylsulfonyl)-3-cinnolinecarboxamide

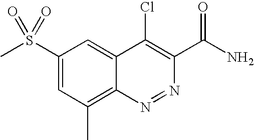

To 4-chloro-8-methyl-6-(methylthio)-3-cinnolinecarboxamide (55 mg) (for example, as prepared for Intermediate 12) in N,N-dimethylformamide (4 ml) was added oxone (340 mg) and the mixture stirred at room temperature for 5.5 hours. The mixture was quenched by the addition of saturated sodium sulphite solution (2 ml) and the resultant solution partitioned between dichloromethane (25 ml) and water (25 ml). The organic layer was collected and the aqueous layer extracted with dichloromethane (20 ml). The combined organic layers were concentrated in vacuo to give the title compound as a dark solid (40 mg), which was a mixture containing ca. 60% by UV of the title compound by LC/MS.

LC/MS Rₜ 2.15 min m/z 300 [MH⁺]; Impurities Rₜ 1.99 min m/z 282 [MH⁺], Rₜ 2.26 min m/z 264 [MH⁺].

### Intermediate 15. 4-[(5-Chloro-3-pyridinyl)amino]-8-methyl-6-(methylthio)-3-cinnolinecarboxamide

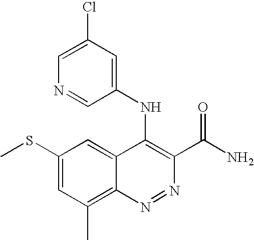

4-Chloro-8-methyl-6-(methylthio)-3-cinnolinecarboxamide (70 mg) (for example, as prepared for Intermediate 12) was heated at 85° C. with 5-chloro-3-pyridinamine dihydrochloride (58 mg; available from PharmLab Product List; see also Roczniki Chemii (1968), 42(12), 2079-88) in acetonitrile (5 ml) for 18 h. Pyridine hydrochloride (30 mg) was added and heating continued at 85° C. for 24 h. DMF (0.5 ml) was added, and heating continued at 85° C. for 5 h. More 5-chloro-3-pyridinamine dihydrochloride (26 mg) and pyridine hydrochloride (30 mg) were added, and heating was continued at 85° C. for 18 h. The mixture was cooled to room temperature, filtered and the filtrate washed with acetonitrile and dried in vacuo to give the impure product hydrochloride (90 mg). This was taken up in 1:1 dichloromethane: methanol and loaded onto an aminopropyl SPE cartridge (pre-conditioned with methanol and 1:1 dichloromethane: methanol). The cartridge was eluted with dichloromethane: methanol, then methanol and the fractions evaporated to give the title compound free base as a yellow solid (30 mg).

LC/MS Rt 3.05 min 360 m/z [MH⁺]

Similarly prepared from 4-chloro-8-methyl-6-(methylthio)-3-cinnolinecarboxamide (for example, as prepared for Intermediate 12) and 3,4-dimethyl-5-isoxazolamine (available from Aldrich) was:

### Intermediate 16. 4-[(3,4-Dimethyl-5-isoxazolyl)amino]-8-methyl-6-(methylthio)-3-cinnolinecarboxamide

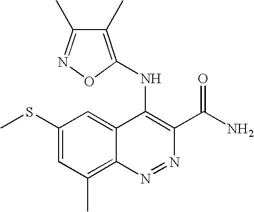

LC/MS Rt 3.01 min m/z 344 [MH⁺]

### Intermediate 17. 4-[(6-Fluoro-5-methyl-3-pyridinyl)amino]-8-methyl-6-(methylthio)-3-cinnolinecarboxamide

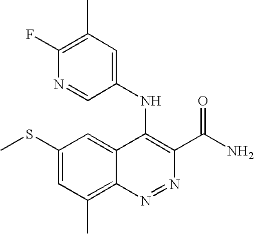

To a suspension of 4-chloro-8-methyl-6-(methylthio)-3-cinnolinecarboxamide (58 mg) (for example, as prepared for Intermediate 12) in acetonitrile (5 ml) was added 6-fluoro-5-methyl-3-pyridinamine (available from Asymchem Laboratories; 33 mg) and the mixture was treated at 85° C. for 16 h. The solid was filtered off, dried and re-dissolved in 1:1 methanol: dichloromethane (5 ml). This solution was loaded onto an aminopropyl SPE cartridge (5 g) pre-conditioned with methanol then 1:1 methanol: dichloromethane. The cartridge was eluted with 1:1 methanol: dichloromethane and methanol, and the product containing fractions evaporated under a stream of nitrogen to give the title compound as a white solid (48 mg).

LC/MS Rt 3.08 min m/z 358 [MH⁺]

Similarly prepared were:

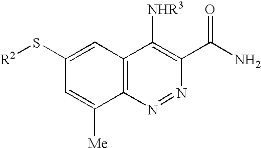

| Intermediate | | | | | | |
|---|---|---|---|---|---|---|
| Number/ | | | | | | LCMS |
| isolation | | | Starting | Amine/Supplier/ | LCMS | Rₜ |
| method | R³NH— | R²S— | Material | reference | MH⁺ | (min) |
| Intermediate 18Free base (a) | 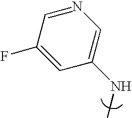 | EtS— | 4-Chloro-6-(ethylthio)-8-methyl-3-cinnolinecarboxamide(for exampleas preparedfor Intermediate13) | 5-fluoro-3-pyridinaminedihydrochloride/MatrixScientific/Journal ofthe Chemical Society,Perkin Transactions 1:Organic and Bio-Organic Chemistry(1998), (10), 1705-1713. | 358 | 3.05 |
| Intermediate 75Free base (c) | 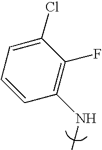 | MeS— | 4-Chloro-6-(methylthio)-3-cinnolinecarboxamide(for example asprepared forIntermediate 27) | 3-chloro-2-fluoroaniline (Aldrich) | 363/365 | 3.28 |
| Intermediate 76Free base (a) | 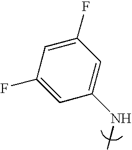 | MeS— | 4-Chloro-6-(methylthio)-3-cinnolinecarboxamide(for example as prepared forIntermediate 27) | 3,5-difluoroaniline/Aldrich | 347 | 3.21 |
| Intermediate 77Free base (b) | 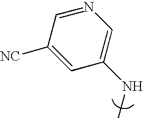 | EtS— | 4-Chloro-6-(ethylthio)-8-methyl-3-cinnolinecarboxamide(for exampleas prepared forIntermediate 13) | 5-amino-3-pyridinecarbonitrile/ChemPacific ProductList/Journal ofMedicinal Chemistry(1967), 10(2), 149-54 | 365 | 2.96 |

(a) isolated by filtration of the HCl salt from the reaction mixture, washing with acetonitrile, and eluting through an aminopropyl SPE cartridge with methanol: dichloromethane.
(b) isolated by filtration of the HCl salt from the reaction mixture, washing with acetonitrile, evaporating the mother liquor material and purifying the residue by flash chromatography on silica gel, eluting with CH₂Cl₂:MeOH:Et₃N. The combined product was then and eluted through an aminopropyl SPE cartridge with methanol: dichloromethane.
(c) isolated by filtration of the HCl salt from the reaction mixture, washing with acetonitrile, followed by purification by flash chromatography on silica gel, eluting with CH₂Cl₂:MeOH:Et₃N.

### Intermediate 19. 2-({[(1,1-Dimethylethyl)oxy]carbonyl}amino)-5-iodobenzoic acid

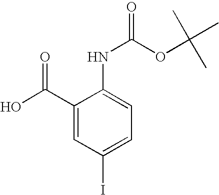

A stirred mixture of 2-amino-5-iodobenzoic acid (available from Aldrich; 10 g) and bis(1,1-dimethylethyl) dicarbonate (10 g) in 1,4-dioxane (50 ml) and 1M sodium hydroxide (50 ml) was stirred at room temperature for 18 h and warmed at 50° C. for 1.5 h. More bis(1,1-dimethylethyl) dicarbonate (4.2 g) in 1,4-dioxane (20 ml) was added along with 1M sodium hydroxide (20 ml), and warming continued at 50° C. for 3 h. The mixture was cooled to room temperature, treated with bis(1,1-dimethylethyl) dicarbonate (4.2 g) in 1,4-dioxane and stirred for 18 h. The mixture was poured into water (400 ml) and the pH adjusted to pH4-5 with 2H hydrochloric acid. The resulting precipitate was collected by filtration, washed with water and dried in vacuo at 45° C. overnight to obtain the title compound as a mauve solid (12.35 g, 89.5%).

LC/MS Rt 4.10 min m/z 362 [M−H]

### Intermediate 20. 1,1-Dimethylethyl (2-acetyl-4-iodophenyl)carbamate

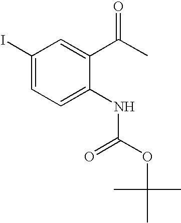

A stirred solution of 2-({[(1,1-dimethylethyl)oxy]carbonyl}amino)-5-iodobenzoic acid (1.09 g) (for example as prepared for Intermediate 19) in anhydrous THF (20 ml) under nitrogen was cooled to 0-5° C. (ice bath). Methylmagnesium bromide (5 ml of a 3M solution in ether) was added over 5-10 min. The cooling bath was removed and stirring was continued for 6 h, followed by stirring at room temperature overnight. The mixture was poured cautiously into water (75 ml) and the pH was adjusted to ca. pH 6 by the addition of 2N hydrochloric acid. Following extraction with ethyl acetate (2×50 ml) the combined organic extracts were washed with saturated sodium bicarbonate (50 ml) and brine (30 ml), dried (Na₂SO₄) and evaporated in vacuo to give a brown oil (1.1 g). Purification by chromatography on silica gel (50 g SPE cartridge), eluting with 0-50% ethyl acetate in cyclohexane over 40 min, gave the title compound as a very pale yellow solid (0.73 g; 67%).

LC/MS Rt 3.77 min m/z 360 [M−H]

### Intermediate 21. 1-(2-Amino-5-iodophenyl)ethanone

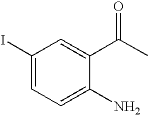

A stirred solution of 1,1-dimethylethyl (2-acetyl-4-iodophenyl)carbamate (0.71 g) (for example as prepared for Intermediate 20) in dichloromethane (10 ml) was treated with trifluoroacetic acid (5 ml), and stirring was continued for 1.5 h. The mixture was concentrated in vacuo and the residue was dissolved in dichloromethane (50 ml). This solution was washed with saturated sodium bicarbonate (50 ml), dried (Na₂SO₄) and evaporated in vacuo to obtain the title compound as a yellow solid (0.50 g; 97%).

LC/MS Rt 3.02 min, m/z 262 [MH⁺]

### Intermediate 22. 1-{5-Iodo-2-[1-pyrrolidinyldiazenyl]phenyl}ethanone (Assumed to be a Mixture of E and Z Isomers)

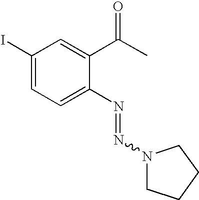

A stirred suspension of 1-(2-amino-5-iodophenyl)ethanone (490 mg) (for example as prepared for Intermediate 21) in 6N hydrochloric acid (3 ml) was cooled to 0-5° C. (ice bath). A solution of sodium nitrite (130 mg) in water (1 ml) was added dropwise resulting in an orange solution containing some insoluble material. After 10 min this diazonium salt mixture was filtered and added dropwise to a cooled (ice bath) stirred solution of pyrrolidine (0.157 ml) in 1.1M potassium hydroxide solution (15 ml) causing a precipitate to form. After 15 min the precipitate was harvested by filtration under reduced pressure, washed with water and dried in vacuo at 50° C. for 5 h to obtain the title compound as a sand-coloured solid (535 mg; 83%).

LC/MS Rt 3.55 min, m/z 344 [MH⁺]

### Intermediate 23. Ethyl 3-{5-iodo-2-[1-pyrrolidinyldiazenyl]phenyl}-3-oxopropanoate (Assumed to be a Mixture of E and Z Isomers)

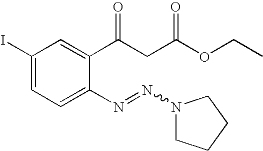

Sodium hydride (180 mg; 60% dispersion in mineral oil) was added to a solution of diethyl carbonate (3.6 ml) in dry THF (7.5 ml). The mixture was heated to reflux, then treated dropwise with a solution of 1-{5-iodo-2-[1-pyrrolidinyldiazenyl]phenyl}ethanone (Intermediate 22; 515 mg) in dry THF (3 ml) over 15 min. After heating at reflux for a further 30 min the mixture was cooled to room temperature and partitioned between saturated aqueous ammonium chloride (50 ml) and ether (40 ml). The layers were separated and the aqueous phase was further extracted with ether (30 ml). The combined organic extracts were dried (Na₂SO₄) and evaporated in vacuo. The residue was triturated with ether to obtain the title compound as a pale beige solid (450 mg).

LC/MS Rt 3.62 min, m/z 416 [MH⁺]

### Intermediate 24. Ethyl 6-iodo-4-oxo-1,4-dihydro-3-cinnolinecarboxylate

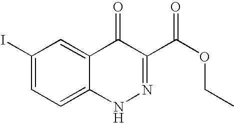

Ethyl 3-{5-iodo-2-[1-pyrrolidinyidiazenyl]phenyl}-3-oxopropanoate (440 mg) (for example as prepared for Intermediate 23) was added in portions to cooled (ice bath) trifluoroacetic acid (3 ml) over 5 min. The resulting orange solution was stirred at room temperature overnight, and added over 5 minutes to ice-cooled water (15 ml), causing a solid to precipitate. This was collected by filtration, washed with water and dried in vacuo at 40° C. to obtain the title compound as a cream-coloured solid (346 mg).

LC/MS Rt 2.69 min, m/z 345 [MH⁺]

### Intermediate 25. 6-Iodo-4-oxo-1,4-dihydro-3-cinnolinecarboxylic acid

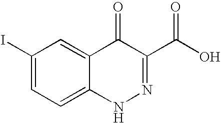

A stirred suspension of ethyl 6-iodo-4-oxo-1,4-dihydro-3-cinnolinecarboxylate (100 mg) (for example as prepared for Intermediate 24) in methanol (2 ml) was treated with 2N sodium hydroxide (0.5 ml). Stirring was continued for 30 min, followed by heating at 50° C. for 1 h. The mixture was cooled to room temperature, then added to 2N hydrochloric acid with stirring. The precipitate was filtered under reduced pressure, washed with water and dried in vacuo at 45° C. to obtain the title compound as a cream coloured solid (84.5 mg; 92%).

LC/MS Rt 2.87 min, m/z 317 [MH⁺]

### Intermediate 26. 4-Chloro-6-iodo-3-cinnolinecarboxamide

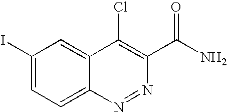

6-Iodo-4-oxo-1,4-dihydro-3-cinnolinecarboxylic acid (3.52 g) (for example as prepared for Intermediate 25) was heated with phosphorous oxychloride (60 ml) at 90° C. with stirring under nitrogen for 2 h. Heating was continued at 100° C. for 4 h. The excess phosphorous oxychloride was evaporated in vacuo, and the residue azeotroped with toluene (60 ml). The resulting dark brown oil was dissolved in dry THF (20 ml) and the solution added slowly to stirred 880 ammonia, with cooling in an ice bath. The mixture was stirred at 0° C. for 1 h, and the resulting precipitate filtered off, washed with water, and dried to give the title compound as a grey solid (3.05 g; 82%).

LC/MS Rt 2.55 min, m/z 334 [MH⁺]

### Intermediate 27. 4-Chloro-6-(methylthio)-3-cinnolinecarboxamide

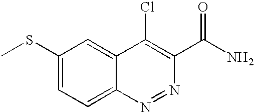

4-chloro-6-iodo-3-cinnolinecarboxamide (1.5 g) (for example as prepared for Intermediate 26) was heated under microwave irradiation with tributyl(methylthio)stannane (1.8 g) (for example as prepared for Intermediate 44) and tetrakis(triphenylphosphine)palladium (0) (252 mg) in N,N-dimethylformamide (20 ml) at 130° C. for 10 min. The solvent was evaporated in vacuo, and the residue was triturated with 50:50 ethyl acetate: cyclohexane (70 ml). The resulting solid was filtered off, washed with 50:50 ethyl acetate: cyclohexane, and dried in vacuo to give the title compound as a brown solid (1.11 g; 94%).

LC/MS Rt 2.42 min, m/z 254 [MH⁺]

### Intermediate 28. 4-Chloro-6-(ethylthio)-3-cinnolinecarboxamide

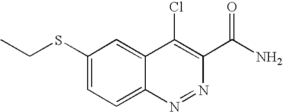

4-chloro-6-iodo-3-cinnolinecarboxamide (700 mg) (for example as prepared for Intermediate 26) was heated under microwave irradiation with tributyl(ethylthio)stannane (925 mg) (for example as prepared for Intermediate 58) and tetrakis(triphenylphosphine)palladium (0) (125 mg) in N,N-dimethylformamide (10 ml) at 130° C. for 10 min. The solvent was evaporated in vacuo to give a dark brown gum which was triturated with 50:50 ethyl acetate: cyclohexane. The resulting grey solid was filtered off, washed with 50:50 ethyl acetate: cyclohexane, and dried in vacuo to give the title compound as a grey solid (484 mg; 82%).

LC/MS Rt 2.66 min, m/z 268 [MH⁺]

### Intermediate 29. 4-Chloro-6-[(11-dimethylethyl)thio]-3-cinnolinecarboxamide

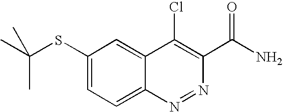

4-chloro-6-iodo-3-cinnolinecarboxamide (0.70 g) (for example as prepared for Intermediate 26) was heated under microwave irradiation with tributyl[(1,1-dimethylethyl)thio]stannane (1.21 g) (for example as prepared for Intermediate 60) and tetrakis(triphenylphosphine)palladium (0) (125 mg) in N,N-dimethylformamide (10 ml) at 130° C. for 10 min. The solvent was evaporated in vacuo, and the residue was triturated with 1:2 ethyl acetate: cyclohexane (20 ml) giving a precipitate. The mixture was diluted with further cyclohexane (ca. 10 ml), and the resulting solid was filtered off to give the title compound as a dark brown solid (0.368 g; 57%).

LC/MS Rt 3.01 min, m/z 296 [MH⁺]

### Intermediate 30. 4-[(3-Cyanophenyl)amino]-6-(methylthio)-3-cinnolinecarboxamide (Assumed to be the Hydrochloride)

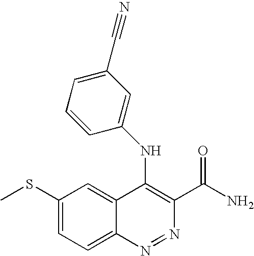

4-chloro-6-(methylthio)-3-cinnolinecarboxamide (Intermediate 27; 64 mg) was heated under reflux with 3-aminobenzonitrile (36 mg) in acetonitrile (15 ml) with stirring under nitrogen for 6 h. The mixture was allowed to cool to room temperature overnight, and the solid filtered off, washed with acetonitrile and dried in vacuo to give the title compound as a brown solid (54 mg; 64%).

LC/MS Rt 2.90 min, m/z 336 [MH⁺]

Similarly prepared were the following:

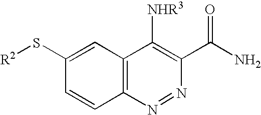

| | | | | | | LCMS |
|---|---|---|---|---|---|---|
| Intermediate | | | Starting | Amine/Supplier/ | LCMS | Rₜ |
| Number | R³NH— | R²S— | Material | reference | MH⁺ | (min) |
| Intermediate31Free base (a) | 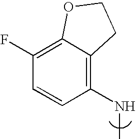 | MeS— | 4-Chloro-6-(methylthio)-3-cinnolinecarboxamide(for example asprepared forIntermediate 27) | 7-fluoro-2,3-dihydro-1-benzofuran-4-amine (for exampleas prepared forIntermediate 50) | 371 | 3.01 |
| Intermediate32Free base (b) | 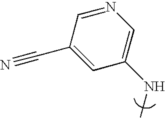 | MeS— | 4-Chloro-6-(methylthio)-3-cinnolinecarboxamide(for example asprepared forIntermediate 27) | 5-amino-3-pyridinecarbonitrile/ChemPacificProduct List/Journalof MedicinalChemistry (1967),10(2), 149-54 | 337 | 2.63 |
| Intermediate34HCl salt (c) | 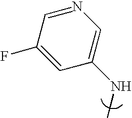 | MeS— | 4-Chloro-6-(methylthio)-3-cinnolinecarboxamide(for example asprepared forIntermediate 27) | 5-fluoro-3-pyridinaminedihydrochloride/Matrix Scientific/Journal of theChemical Society,Perkin Transactions1: Organic and Bio-Organic Chemistry(1998), (10), 1705-1713. | 330 | 2.68 |
| Intermediate35Free base (a) | 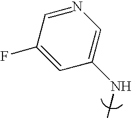 | EtS— | 4-Chloro-6-(ethylthio)-3-cinnolinecarboxamide(for example asprepared forIntermediate 28) | 5-fluoro-3-pyridinaminedihydrochloride/Matrix Scientific/Journal of theChemical Society,Perkin Transactions1: Organic and Bio-Organic Chemistry(1998), (10), 1705-1713. | 344 | 3.0 |
| Intermediate36Free base (a) | 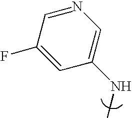 | tBuS— | 4-Chloro-6-[(1,1-dimethylethyl)thio]-3-cinnolinecarboxamide(for exampleas preparedforIntermediate 29) | 5-fluoro-3-pyridinaminedihydrochloride/Matrix Scientific/Journal of theChemical Society,Perkin Transactions1: Organic and Bio-Organic Chemistry(1998), (10), 1705-1713. | 372 | 3.12 |
| Intermediate37 | 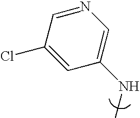 | MeS— | 4-Chloro-6-(methylthio)-3-cinnolinecarboxamide(for example asprepared forIntermediate 27) | 5-chloro-3-pyridinaminedihydrochloride/PharmLab ProductList/Roczniki Chemii(1968), 42(12),2079-88. | 346/348 | 2.86 |
| Intermediate38Free base (a) | 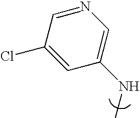 | EtS— | 4-Chloro-6-(ethylthio)-3-cinnolinecarboxamide(for example asprepared forIntermediate 28) | 5-chloro-3-pyridinaminedihydrochloride/PharmLab ProductList/Roczniki Chemii(1968), 42(12),2079-88. | 360/362 | 3.17 |
| Intermediate41Free base (a) | 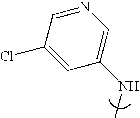 | tBuS— | 4-Chloro-6-[(1,1-dimethylethyl)thio]-3-cinnolinecarboxamide(for exampleas preparedforIntermediate 29) | 5-chloro-3-pyridinaminedihydrochloride/PharmLab ProductList/Roczniki Chemii(1968), 42(12),2079-88. | 388/390 | 3.26 |
| Intermediate76Free base (a) | 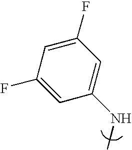 | MeS— | 4-Chloro-6-(methylthio)-3-cinnolinecarboxamide(for example asprepared forIntermediate 27) | 3,5-difluoroaniline/Aldrich | 347 | 3.21 |

(d) Isolated by filtration from the reaction mixture, applying to an aminopropyl SPE cartridge and eluting with a mixture of CH₂Cl₂/MeOH.
(e) Isolated by filtration from the reaction mixture and purification by flash chromatography on silica gel, eluting with a methanol/triethylamine/dichloromethane gradient.
(f) HCl salts isolated by filtration from the reaction mixture and washing with acetonitrile.

### Intermediate 33. 4-[(1-Ethyl-1H-pyrazol-5-yl)amino]-6-(methylthio)-3-cinnolinecarboxamide

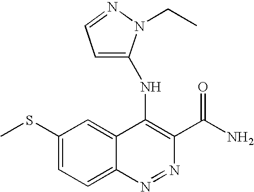

4-chloro-6-(methylthio)-3-cinnolinecarboxamide (150 mg) (for example as prepared for Intermediate 27) was heated under reflux with 1-ethyl-1H-pyrazol-5-amine (available from Aldrich; 79 mg) in acetonitrile (20 ml) with stirring under nitrogen for 16 h. Pyridine hydrochloride (103 mg) was added and heating under reflux was continued for 18 h. The mixture was evaporated in vacuo, and the residue dissolved in dichloromethane/methanol. Florisil (ca. 10 g) was added and the solvents evaporated to give a brown powder, which was applied to a 50 g silica gel chromatography column. Elution with dichloromethane: methanol: triethylamine (using a gradient containing 0-15% methanol and 0-0.15% triethylamine in dichloromethane over 40 min) gave the title compound (127 mg; 67%).

LC/MS Rt 2.62 min, m/z 329 [MH⁺]

Similarly prepared were the following:

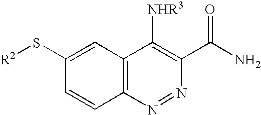

| | | | | | | LCMS |
|---|---|---|---|---|---|---|
| Intermediate | | | Starting | Amine/Supplier/ | LCMS | Rₜ |
| Number | R³NH— | R²S— | Material | reference | MH⁺ | (min) |
| Intermediate39Free base (a) | 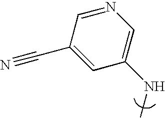 | EtS— | 4-Chloro-6-(ethylthio)-3-cinnolinecarboxamide(for example asprepared forIntermediate 28) | 5-amino-3-pyridinecarbonitrile/ChemPacific ProductList/Journal ofMedicinal Chemistry(1967), 10(2), 149-54 | 351 | 2.81 |
| Intermediate40Free base (a) | 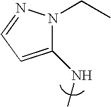 | EtS— | 4-Chloro-6-(ethylthio)-3-cinnolinecarboxamide(for example asprepared forIntermediate 28) | 1-ethyl-1H-pyrazol-5-amine/Aldrich | 343 | 2.84 |
| Intermediate42Free base (a) | 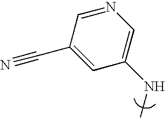 | tBuS— | 4-Chloro-6-[(1,1-dimethylethyl)thio]-3-cinnolinecarboxamide(for exampleas preparedforIntermediate 29) | 5-amino-3-pyridinecarbonitrile/ChemPacific Product List/Journal ofMedicinal Chemistry(1967), 10(2), 149-54 | 379 | 3.19 |
| Intermediate43Free base (b) | 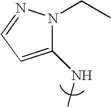 | tBuS— | 4-Chloro-6-[(1,1-dimethylethyl)thio]-3-cinnolinecarboxamide(for exampleas preparedforIntermediate 29) | 1-ethyl-1H-pyrazol-5-amine/Adrich | 371 | 3.05 |

(a) Isolated by filtration from the reaction mixture, applying to an aminopropyl SPE cartridge and eluting with 50/50 CH₂Cl₂: MeOH.
(b) Isolated by filtration from the reaction mixture, eluting through an aminopropyl SPE cartridge and purification by flash chromatography on silica gel, eluting with a methanol/triethylamine/dichloromethane gradient.

### Intermediate 44. Tributyl(methylthio)stannane

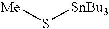

To a solution of tributyltin chloride (82 g) in anhydrous carbon tetrachloride (500 ml) was added sodium thiomethoxide (19.4 g, available from Aldrich) and the resulting mixture was stirred for 72 h under nitrogen. The mixture was filtered through Celite® filter agent and concentrated in vacuo to give the title compound as a colourless oil (88.4 g; 95%).

¹H NMR (400 MHz, CDCl₃) δ 0.92 (t, J=7 Hz, 9H), δ 1.15 (m, J=7 Hz, 6H), δ 1.35 (m, J=7 Hz, 6H), δ 1.58 (m, J=7 Hz, 6H), δ 2.09 (m, J=14 Hz, 3H).

### Intermediate 45. 2-Methoxy-4-nitrobenzenediazonium tetrafluoroborate

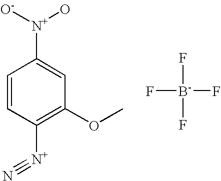

To a suspension of 2-methoxy-4-nitroaniline (249.5 g; available from Aldrich) in water (200 ml) was added concentrated hydrochloric acid (400 ml) and the mixture heated at 95° C. for 1 hour. The mixture was cooled (ice/methanol) and a solution of sodium nitrite (108.5 g) in water (350 ml) was added dropwise at 0° C. over a period of 2 hours. The mixture was stirred for 45 minutes to give a solution. Tetrafluoroboric acid (270 g) was added at 0° C. over 1 minute and the mixture stirred at 0° C. for 30 minutes. The solid was collected, washed with water (100 ml), methanol (100 ml), and diethyl ether (100 ml), then dried (over concentrated sulphuric acid) to give the title compound as a yellow solid (179.3 g).

NMR: (300 MHz, d-6 DMSO) δ 8.79 (J=9 Hz; 1H,d), δ 8.35 (J=2 Hz; 1H,d), δ 8.16 (J=9, 2 Hz; 1H,dd), δ 4.31 (3H,s).

### Intermediate 46. 1-Fluoro-2-(methyloxy)-4-nitrobenzene

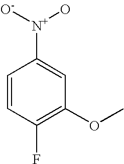

2-Methoxy-4-nitrobenzenediazonium tetrafluoroborate (179.3 g) (for example as prepared for Intermediate 45) and sand (150 g) were placed in a 1 l round-bottomed-flask (RBF) that was fitted with a gas exhaust connected to a gas inlet which was placed into water (200 ml) in a 3-necked 1 l RBF that was cooled in ice. The exhaust from this flask led to a second 3-necked 500 ml RBF containing water (100 ml). The flask containing Intermediate 45 was heated with a heat-gun. The mixture was allowed to cool. The flask containing the sand was extracted with diethyl ether (500 ml) and ethereal extracts combined with the other flask contents. The mixture was filtered and the aqueous layer separated. The aqueous layer was extracted with diethylether (500 ml). The combined organic extracts were evaporated and the residue solidified on standing to give the title compound as a red solid (29.8 g).

NMR: (300 MHz, CDCl₃) δ 7.85 (2H,m), 6 7.20 (J=8 Hz; 1H,t), δ 3.98 (3H,s).

### Intermediate 47. 4-Fluoro-3-methoxyaniline

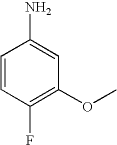

To a solution of 1-fluoro-2-(methyloxy)-4-nitrobenzene (32.6 g) (for example as prepared for Intermediate 46) in methanol (300 ml) in an autoclave was added 5% palladium on carbon (1 g) in toluene (2 ml). The autoclave was purged with argon, then stirred under 50 atmospheres of hydrogen overnight. The mixture was filtered through celite and the filtrate evaporated. The residue was dissolved in ethyl acetate (250 ml), then filtered. The filtrate was extracted with 2M hydrochloric acid (200 ml). The aqueous phase was made basic by the addition of 2M sodium hydroxide (300 ml) and extracted with ethyl acetate (250 ml). The organic layer was washed with water (100 ml) and brine (100 ml) (with filtering), further washed with brine (200 ml) and evaporated to give the title compound as a red liquid (18.3 g).

NMR (300 MHz, CDCl₃) δ 6.84 (J=9,11 Hz; 1H,dd), δ 6.30 (1H,m), δ 6.15 (J=3,9 Hz; 1H,dt), δ 3.82 (3H,s).

### Intermediate 48. N-[4-Fluoro-3-(methyloxy)phenyl]-2,2-dimethylpropanamide

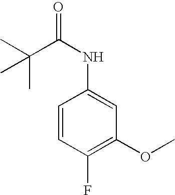

To a solution of 4-fluoro-3-methoxyaniline (18.3 g) (for example as prepared for Intermediate 47) in dichloromethane (200 ml) was added triethylamine (18.0 ml). The solution was cooled to 0° C. and 2,2-dimethylpropanoyl chloride (16.0 ml) added dropwise. The mixture was stirred at room temperature overnight. The mixture was washed with water (200 ml), dried over magnesium sulphate, filtered through silica and concentrated. Hexane was added and the solution concentrated. The resultant solid was washed with hexane and then dried to give the title compound as a white solid (16.1 g).

NMR: (300 MHz, CDCl₃) δ 7.64 (J=2,9 Hz; 1H,dd), δ 6.99 (J=11,9 Hz; 1H,dd), δ 6.71 (J=2,4,9 Hz; 1H,ddd), δ 3.90 (3H,s), δ 1.32 (9H,s).

### Intermediate 49. N-[4-Fluoro-2-(2-hydroxyethyl)-3-(methyloxy)phenyl]-2,2-dimethylpropanamide

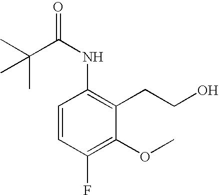

To a suspension of N-[4-fluoro-3-(methyloxy)phenyl]-2,2-dimethylpropanamide (16.0 g) (for example as prepared for Intermediate 48) in tetrahydrofuran (150 ml) at −10° C., under argon, was added 2.5M n-butyl lithium in hexane (71 ml) over 40 min keeping the temp below 0° C. The mixture was stirred at 0° C. for 1 hour, then cooled to −10° C. and a solution of ethylene oxide in tetrahydrofuran (20 ml) added over 20 minutes. The mixture was stirred at room temperature for 3 hours. The reaction was quenched with 880 ammonia (10 ml) in water (100 ml) with cooling. Ethyl acetate (100 ml) was added. The organic layer was separated, washed with a mixture of water (100 ml) and brine (50 ml), further washed with brine (100 ml) and the solvent evaporated. Recrystallisation from diethyl ether/hexane gave the title compound as a white solid (14.0 g).

NMR (300 MHz; CDCl₃) δ 8.57 (1H,bs), δ 7.42 (J=5,9 Hz; 1H,dd), δ 6.97 (J=11,9 Hz; 1H,dd), δ 3.93 (J=5 Hz; 2H,t), δ 3.89 (J=2 Hz; 3H,d), δ 2.86 (J=5 Hz; 2H,t), δ 2.02 (1H,bs), δ 1.29 (9H,s).

### Intermediate 50. 7-Fluoro-2,3-dihydro-1-benzofuran-4-amine p-toluenesulfonate salt

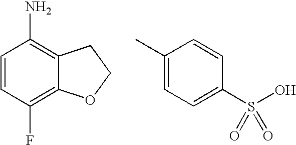

A mixture of N-[4-fluoro-2-(2-hydroxyethyl)-3-(methyloxy)phenyl]-2,2-dimethylpropanamide (14.0 g) (for example as prepared for Intermediate 49) in 48% aqueous hydrobromic acid (50 ml) was heated at reflux overnight. The solution was cooled, dichloromethane (100 ml) was added, then 5M sodium hydroxide solution (120 ml) was added with cooling. The aqueous phase was extracted with dichloromethane (2×50 ml) and the combined organic layers were evaporated. The residue was dissolved in ethyl acetate (100 ml), washed with water (50 ml), then brine (50 ml), dried (MgSO₄ with charcoal), filtered through a thin pad of silica and then evaporated. The residue was dissolved in ethyl acetate (7 ml) and a warm solution of para-toluenesulphonic acid (4.1 g) in ethyl acetate (15 ml) was added. The solid was collected, washed with ethyl acetate and dried to give the title compound as a white solid (6.0 g).

NMR (300 MHz; CDCl₃) δ 7.44 (J=8 Hz; 2H,d), δ 7.09 (J=8 Hz; 2H,d), δ 7.03 (1H, m), δ 6.64 (J=3,9 Hz; 1H,dd), δ 4.66 (J=9 Hz; 2H,t), δ 3.20 (J=9 Hz; 2H,t), δ 2.25 (3H, s).

### Intermediate 51. 2-Amino-5-iodo-3-methylbenzoic acid

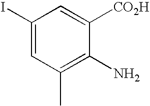

A stirred solution of 2-amino-3-methylbenzoic acid (available from Aldrich; 50.1 g, 0.33 mol) in 1M hydrochloric acid (1 l) was maintained at ambient temperature. An ice-cooled solution of iodine monochloride (64.5 g, 0.4 mol) in 2M hydrochloric acid (200 ml) was added at a constant rate over 30 mins. After stirring for a further 1.5 h the mixture was filtered under reduced pressure and the solid washed with water and dried in vacuo at 50° C. The resulting pale beige solid was treated with acetonitrile (1 l) and heated at 80° C. for 15 mins. The mixture was allowed to cool to 50° C. and filtered under reduced pressure. The solid was washed with acetonitrile and dried in vacuo at 50° C. to obtain the title compound as a cream coloured solid (71.5 g, 78%).

LC/MS Rₜ 3.23 min m/z 276 [M−H].

### Intermediate 52. 5-Iodo-3-methyl-2-[(trifluoroacetyl)amino]benzoic acid

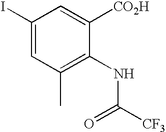

To a stirred solution of 2-amino-5-iodo-3-methylbenzoic acid (66 g, 0.238 mol) (for example as prepared for Intermediate 51) in 1,4-dioxane (500 ml) was added trifluoroacetic anhydride (50.6 ml, 0.357 mol). The solution was cooled to ca. 15° C. and treated with 1M sodium hydroxide (500 ml) resulting in an exotherm (reaction temp rose to 30° C.). After stirring for 2 h the cloudy solution was poured into water (1 l). The pH was adjusted to pH2-3 with 2M hydrochloric acid then the mixture was extracted with ethyl acetate (2×500 ml). The combined organic extracts were dried (Na₂SO₄) then evaporated in vacuo to a very pale pink solid. This was triturated with cyclohexane to obtain the title compound as a cream coloured solid (62.4 g, 70%).

### LC/MS Rₜ 3.60 min m/z 372 [M−H].

### Intermediate 53. N-(2-acetyl-4-iodo-6-methylphenyl)-2 2,2-trifluoroacetamide

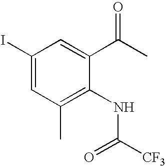

A stirred solution of 5-iodo-3-methyl-2-[(trifluoroacetyl)amino]benzoic acid (60 g, 0.161 mol) (for example as prepared for Intermediate 52) in anhydrous THF (800 ml), under a nitrogen atmosphere, was cooled to 0° C. Methylmagnesium chloride (268 ml of a 3M solution in THF, 0.805 mol) was added over 40 mins maintaining the reaction temperature below 10° C. (addition of the first two equivalents caused an exotherm). The cooling bath was removed and stirring was continued for 3.5 h. The mixture was poured cautiously onto crushed ice, the pH was adjusted to pH2-3 with 2M hydrochloric acid, then extracted twice with ethyl acetate. The combined organic extracts were dried (Na₂SO₄) then evaporated in vacuo . The residue was triturated with isopropyl ether to obtain the title compound as a cream coloured solid (43.5 g, 73%).

LC/MS Rₜ 3.17 min m/z 370 [M−H].

### Intermediate 54. 1-(2-Amino-5-iodo-3-methylphenyl)ethanone

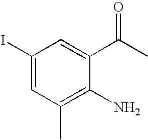

To a stirred suspension of N-(2-acetyl-4-iodo-6-methylphenyl)-2,2,2-trifluoroacetamide (43 g, 0.116 mol) (for example as prepared for Intermediate 53) in methanol (200 ml) was added 2M sodium hydroxide (200 ml). The resulting solution was heated at reflux for 3 h causing a precipitate. After cooling to room temperature the mixture was poured into water (500 ml) and filtered. The filtered solid was washed with water and dried in vacuo to obtain the title compound as a yellow solid (30.3 g, 95%).

LC/MS Rₜ 3.26 min m/z 276 [MH⁺].

### Intermediate 55. 1-{5-Iodo-3-methyl-2-[1-pyrrolidinyldiazenyl]phenyl}ethanone (assumed to be a Mixture of E and Z Isomers)

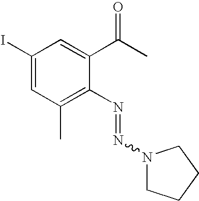

A stirred solution/suspension of 1-(2-amino-5-iodo-3-methylphenyl)ethanone (28.43 g, 0.103 mol) (for example as prepared for Intermediate 54) in THF (200 ml) and water (20 ml) was cooled to 0-5° C. Conc. hydrochloric acid (43.4 ml, 0.515 mol) was added, followed by dropwise addition of an ice-cooled solution of sodium nitrite (8.62 g, 0.124 mol) in water (60 ml) dropwise over 15 mins. After stirring at 0-5° C. for 30 mins the yellow/orange solution was added to an ice-cooled solution of pyrrolidine (16 ml, 0.206 mol) in saturated aqueous potassium carbonate (400 ml) over 30 mins. Once the addition was complete ether (200 ml) was added and the biphasic mixture was stirred vigorously. The layers were separated and the aqueous layer was further extracted with ether (200 ml). The combined organic extracts were washed with 1M hydrochloric acid (200 ml), dried (Na₂SO₄) then evaporated in vacuo to give an orange/brown oil which solidified on standing. This was triturated with isopropanol to obtain the title compound as an orange/brown solid (31.5 g, 85%).

LC/MS Rₜ 3.58 min m/z 358 [MH⁺].

### Intermediate 56. Ethyl 3-{5-iodo-3-methyl-2-[1-pyrrolidinyldiazenyl]phenyl}-3-oxopropanoate (Assumed to be a Mixture of E and Z Isomers)

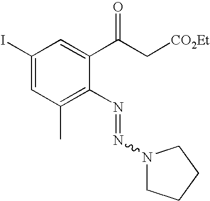

Sodium hydride (10.4 g of a 60% dispersion in mineral oil, 0.26 mol) was added to a solution of diethyl carbonate (210 ml, 1.74 mol) in anhydrous THF (380 ml) under nitrogen. The mixture was heated to reflux then treated with a solution of 1-{5-iodo-3-methyl-2-[(E)-1-pyrrolidinyldiazenyl]phenyl}ethanone (31 g, 0.087 mol) (for example as prepared for in Intermediate 55) anhydrous THF (170 ml) over 45 mins. Heating at reflux was continued for 30 mins then the mixture was cooled to room temperature and partitioned between saturated aqueous ammonium chloride (1 l) and ether (300 ml). The aqueous layer was further extracted with ether (300 ml) then the combined organic extracts were dried (Na₂SO₄) and evaporated in vacuo to give a brown oil. This was triturated with isopropyl ether to obtain the title compound as a tan-coloured solid (26.5 g, 71%).

LC/MS Rₜ 3.69 min m/z 430 [MH⁺].

### Intermediate 57. Ethyl 6-iodo-8-methyl-4-oxo-1,4-dihydro-3-cinnolinecarboxylate

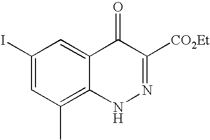

Ethyl 3-{5-iodo-3-methyl-2-[(E)-1-pyrrolidinyidiazenyl]phenyl}-3-oxopropanoate (26.4 g, 0.061 mol) (for example as prepared for Intermediate 56) was added portionwise to ice-cooled trifluoroacetic acid (100 ml) with stirring over 15 mins. The resulting dark brown solution was stirred for 1 h then poured slowly into ice-cooled water (1 l) with stirring. After 15 mins the precipitate was filtered under reduced pressure, washed with water and dried in vacuo at 50° C. to obtain the title compound as a sand-coloured solid (21.9 g, 100%).

LC/MS Rₜ 2.79 min m/z 359 [MH⁺].

### Intermediate 58. Tributyl(ethylthio)stannane

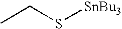

To a solution of ethanethiol (available from Aldrich, 6.8 ml) in dry carbon tetrachloride (250 ml) under nitrogen was added triethylamine (15 ml) with stirring for 10 mins when tributyltin chloride (22.4 ml) was added dropwise over 10 mins. The mixture was stirred at room temperature for 96 h, chloroform was added, and the mixture was filtered through Celite. The filtrate was washed with 5% acetic acid (100 ml) and water (100 ml) then evaporated to give the title compound as a yellow low melting solid (25.1 g).

¹H NMR (400 MHz, CDCl₃): 2.65-2.53 delta (2H, m, CH₂), 1.70-1.48 delta (6H, m, 3xCH₂), 1.40-1.26 delta (9H, m, 3xCH₂+CH₃), 1.23-1.05 delta (6H, m, 3xCH₂), 0.91 delta (9H, t, 3xCH₃)

### Intermediate 59. Tributyl(propylthio)stannane

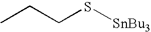

To a solution of 1-propanethiol (available from Aldrich, 4.76 ml) in dry carbon tetrachloride (250 ml) under nitrogen was added triethylamine (8.77 ml). After 10 mins tributyltin chloride (14.3 ml) was added dropwise over 10 mins. The mixture was stirred at room temperature overnight, filtered, and the filtrate was washed with 5% acetic acid (200 ml). The organic layer was separated by hydrophobic frit and concentrated in vacuo to give the title compound as a yellow oil which solidified on standing (18.4 g).

¹H NMR (400 MHz, CDCl₃): 2.57-2.48 delta (2H, m, CH₂), 1.69-1.53 delta (8H, m, 4xCH₂), 1.41-1.29 delta (6H, m, 3xCH₂), 1.22-1.04 delta (6H, m, 3xCH₂), 0.99 delta (3H, t, CH₃), 0.91 delta (9H, t, 3xCH₃).

### Intermediate 60. Tributyl[(1,1-dimethylethyl)thio]stannane

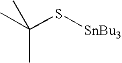

To a solution of 2-methyl-2-propanethiol (available from Aldrich, 1.7 ml) and triethylamine (2.53 ml) in dry carbon tetrachloride (100 ml) under nitrogen was added tributyltin chloride (4.1 ml) dropwise over 20 mins, with vigorous stirring. The mixture was stirred for 18 h at room temperature (20° C.), then filtered through a Celite cartridge (10 g) and the filtrate washed with 5% acetic acid (100 ml) and water (100 ml). The organic layers were separated by hydrophobic frit and concentrated to give the title compound as a colourless oil which slowly solidified (5.2 g).

¹H NMR (400 MHz, CDCl₃) 1.62-1.52 delta (2H, m, CH₂), 1.45 delta (9H, s, 3xCH₃), 1,40-1.30 delta (6H, m, 3xCH₂), 1.24-1.07 delta (6H, m, 3xCH₂), 0.91 delta (9H, 3xCH₃).

### Intermediate 61. 4-[(5-Chloro-3-pyridinyl)amino]-6-(ethylthio)-8-methyl-3-cinnolinecarboxamide hydrochloride

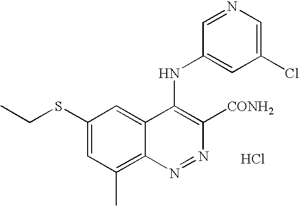

To a suspension of 4-chloro-6-(ethylthio)-8-methyl-3-cinnolinecarboxamide (13; 0.067 g) (for example as prepared for Intermediate 13) in acetonitrile (10 ml) was added 5-chloro-3-pyridinamine dihydrochloride (0.053 g; available from PharmLab Product List; see also Roczniki Chemii (1968), 42(12), 2079-88) and the mixture heated to 90° C. for 18 h. The product was collected by filtration to give the title compound as a yellow solid (0.092 g).

LC/MS Rₜ 3.21 min m/z 374 [MH⁻].

### Intermediate 62. 3-Fluoro-2-[(trifluoroacetyl)amino]benzoic acid

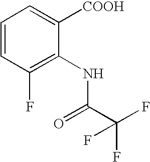

To a solution of 2-amino-3-fluorobenzoic acid (5 g, available from AstaTech) in tetrahydrofuran (60 ml) was added trifluoroacetic anhydride (10.16 g) and the mixture cooled (ice/water), when 1M sodium hydroxide solution (60 ml) was added slowly over approximately 15 mins. The mixture was allowed to stir for 18 h at room temperature then extracted with dichloromethane (100 ml), and the organic layer separated by hydrophobic frit and evaporated in vacuo to give the title compound as a pale brown solid (7.5 g; 93%).

LC/MS Rₜ 2.75 min m/z 250 [M−H].

### Intermediate 63. N-(2-Acetyl-6-fluorophenyl)-2,2.2-trifluoroacetamide

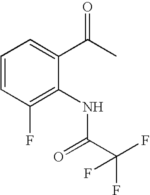

To a solution of 3-fluoro-2-[(trifluoroacetyl)amino]benzoic acid (7.5 g) (for example as prepared for Intermediate 62) in tetrahydrofuran (100 ml) under nitrogen, cooled by ice/water, was added methylmagnesium chloride (3M solution in tetrahydrofuran; 34.9 ml) dropwise over 20 mins. The mixture was allowed to return to room temperature over 18 h and the mixture extracted with dichloromethane (100 ml), separated by hydrophobic frit and the organic layer concentrated in vacuo to give a pale brown solid (0.2 g). The aqueous phase was acidified to pH 1 using 2M hydrochloric acid and extracted with dichloromethane (100 ml). The organic layers were washed with 5% sodium bicarbonate solution (100 ml), separated by hydrophobic frit and concentrated in vacuo to give a pale brown solid (4.3 g). The product solids were combined to give the title compound (total yield 4.5 g; 61%).

LC/MS Rₜ 2.61 min m/z 250 [MH⁺].

### Intermediate 64. 1-(2-Amino-3-fluorophenyl)ethanone

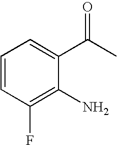

A solution of N-(2-acetyl-6-fluorophenyl)-2,2,2-trifluoroacetamide (0.41 g) (for example as prepared for Intermediate 63) in methanol (5 ml) and 2N sodium hydroxide (5 ml) was heated at 80° C. The mixture was heated for 3 h then left at room temperature for 72 h. The mixture was poured onto water (20 ml), extracted with ethyl acetate (3×25 ml), and the organic layer dried (Na₂SO₄) and evaporated. The crude product was purified by flash chromatography on silica gel (10 g SPE cartridge), eluting with a gradient of 0-50% dichloromethane in cyclohexane to give the title compound as a yellow oil, which solidified on standing (0.15 g, 60%).

LC/MS Rₜ 2.48 min m/z 154 [MH⁺].

### Intermediate 65. 1-(2-Amino-3-fluoro-5-iodophenyl)ethanone

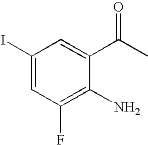

To 1-(2-amino-3-fluorophenyl)ethanone (0.41 g) (for example as prepared for Intermediate 64) was added 1M hydrochloric acid (15 ml) and the heterogeneous mixture was stirred at room temperature. To this mixture was added an ice-cold solution of iodine monochloride (0.47 g) in 2M hydrochloric acid (5 ml) dropwise over ca. 3 min, resulting in a yellow precipitate. After stirring for a further 2.5 h at room temperature, the mixture was filtered and the solid washed with water, sucked dry under vacuum and dried in vacuo at 50° C. for 18 h to give the title compound as a yellow solid (0.67 g, 92%). LC/MS R^{t} 3.13 min m/z 280 [MH⁺].

### Intermediate 66 1-{3-Fluoro-5-iodo-2-[(E)-1-pyrrolidinyldiazenyl]phenyl}ethanone. (Assumed to be a Mixture of E and Z Isomers)

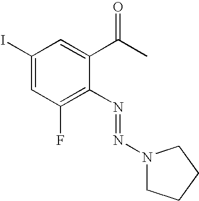

To a stirred suspension of 1-(2-amino-3-fluoro-5-iodophenyl)ethanone (1.51 g) (for example as prepared for Intermediate 65) in 6N hydrochloric acid (10 ml) at 0-5° C. (ice-water) was added dropwise, a solution of sodium nitrite (0.37 g) in water (5 ml) over 15 mins, maintaining a temperature at around 0° C. The cloudy yellow solution was added, cold, to a cold (ice bath) solution of pyrrolidine (0.45 ml) in 1.1M potassium hydroxide solution over 15 mins, maintaining the temperature between −5° C. and 0° C. The resultant viscous, dark purple mixture was maintained with stirring at ca. 0° C. for 2 h and the product collected by filtration, washed with water, sucked dry under vacuum for 1.5 h and further dried under vacuum (vacuum oven) at 50° C. for 16 h to give a deep purple/red solid (1.6 g). The crude was purified by flash chromatography on silica gel (100 g cartridge), eluting with a gradient of 0-100% dichloromethane in cyclohexane, to give a red oil, which solidified on standing to give the title compound (1.44 g). The aqueous filtrate was extracted with dichloromethane and the organic layer separated by hydrophobic frit then evaporated to give a deep purple/red gum. This was purified by flash chromatography on silica gel (20 g cartridge), eluting with a gradient of 0-100% dichloromethane in cyclohexane to give further title compound as a yellow oil, which solidified on standing (0.1 g).

LC/MS Rₜ 3.38 min m/z 362 [MH⁺].

### Intermediate 67. Ethyl 8-fluoro-6-iodo-4-oxo-1,4-dihydro-3-quinolinecarboxylate

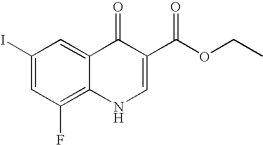

To a stirred solution of diethyl carbonate (10.2 ml) in dry tetrahydrofuran (20 ml) under nitrogen was added sodium hydride (60% dispersion in mineral oil; 0.57 g) in four portions over around 3 mins. The mixture was heated to reflux when a solution of 1-{3-fluoro-5-iodo-2-[(E)-1-pyrrolidinyldiazenyl]phenyl}ethanone (1.52 g) (for example as prepared for Intermediate 66) in tetrahydrofuran (15 ml) was added over 20 mins and the mixture heated at reflux for 4 h. The cooled mixture was diluted with diethyl ether (50 ml) and cautiously poured into saturated ammonium chloride (30 ml). The organic layer was separated and the aqueous layer extracted with diethyl ether (2×50 ml); the combined organic layers were dried (Na₂SO₄) then evaporated to dryness to give a brown gum (1.91 g). The crude product was dissolved in dichloromethane (15 m) and added dropwise to cold (ice/water bath) trifluoroacetic acid (7 ml) over 10 mins. Stirring was continued at ice/water temperature for 20 mins then allowed to warm to room temperature over 1.5 h. The mixture was evaporated to dryness and co-evaporated with dichloromethane then diethyl ether. The crude product was triturated with ether/cyclohexane but failed to solidify, hence the dark brown sludge was purified by loading in dichloromethane onto a silica gel SPE cartridge (50 g), and eluting with a gradient of cyclohexane and ethyl acetate. The product was obtained from 1:1 cyclohexane: ethyl acetate, which was evaporated to give a dark brown solid. This solid was triturated with diethyl ether, filtered, washed with more ether and dried to give the title compound as a pink/brown solid (0.92 g; 60%).

LC/MS Rₜ 2.71 min m/z 363 [MH⁺].

### Intermediate 68. 8-Fluoro-6-iodo-4-oxo-1,4-dihydro-3-quinolinecarboxylic acid

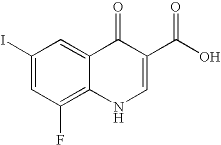

To a suspension of ethyl 8-fluoro-6-iodo-4-oxo-1,4-dihydro-3-quinolinecarboxylate (0.92 g) (for example as prepared for Intermediate 67) in methanol (20 ml) was added 2N sodium hydroxide solution (6 ml) and the mixture heated to 55° C. for 75 min. The cooled mixture was poured onto 2N hydrochloric acid (20 ml) and the precipitate was collected by filtration, washed with water then diethyl ether and dried under vacuum at 45° C. for 20 h to give the title compound as a beige solid (0.786 g; 92%).

LC/MS Rₜ 3.45 min m/z 335 [MH⁺].

### Intermediate 69. 4-Chloro-8-fluoro-6-iodo-3-cinnolinecarboxamide

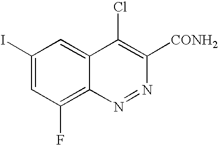

A suspension of 8-fluoro-6-iodo-4-oxo-1,4-dihydro-3-quinolinecarboxylic acid (0.78 g) (for example as prepared for Intermediate 68) in phosphorus oxychloride (10 ml) was heated under nitrogen, at 90° C. for 6.5 h then allowed to cool to room temperature overnight. The phosphorus oxychloride was evaporated and the mixture co-evaporated with dry toluene. The residue was taken up in dry tetrahydrofuran (15 ml) and added dropwise to cold (ice/water) 880 ammonia solution with vigorous stirring over a period of 15 mins. The mixture was stirred for 1 h and the precipitate collected by filtration, washed with water then diethyl ether and dried in vacuo at 45° C. to give a brown solid (0.336 g). The crude product suspended in dichloromethane/methanol (100 ml) and pre-absorbed onto Florisil. Purification by flash chromatography on silica gel (70 g cartridge), eluting with a gradient of 0-100% ethyl acetate in cyclohexane, gave the title compound as a yellow solid (0.13 g).

LC/MS Rₜ 2.53 min m/z 352 [MH⁺].

The filtrate was extracted with dichloromethane (3×50 ml) and the organic layers separated and evaporated to dryness. The residue was purified by flash chromatography on silica gel (70 g cartridge), eluting with a gradient of 0-100% ethyl acetate in cyclohexane, to give further title compound as a brown solid (0.204 g). LC/MS Rₜ 2.53 min m/z 352 [MH⁺].

### Intermediate 70. 4-Chloro-6-(ethylthio)-8-fluoro-3-cinnolinecarboxamide

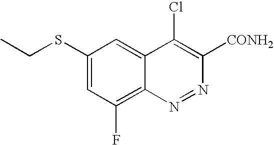

To a solution of 4-chloro-8-fluoro-6-iodo-3-cinnolinecarboxamide (0.2 g) (for example as prepared for Intermediate 69) in DMF (5 ml) was added tributyl(ethylthio)stannane (Intermediate 58; 0.22 g) and tetrakistriphenylphosphine palladium(0) (0.033 g). The mixture was heated under microwave irradiation for 10 mins at 130° C. The mixture was evaporated in vacuo and the residue taken up in dichloromethane, and purified by flash chromatography on silica gel (50 g cartridge), eluting with a gradient of dichloromethane/ethyl acetate to give the title compound as a brown solid (0.088 g; 54%).

LC/MS Rₜ 2.70 min m/z 286 [MH⁺].

### Intermediate 71. 6-(Ethylthio)-8-fluoro-4-[(5-fluoro-3-pyridinyl)amino]-3-cinnolinecarboxamide

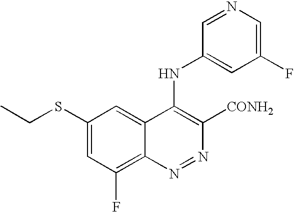

A solution of 4-chloro-6-(ethylthio)-8-fluoro-3-cinnolinecarboxamide (0.088 g) (for example as prepared for Intermediate 70) and 3-amino-5-fluoropyridine dihydrochloride (0.057 g, available from Matrix Scientific; see also Journal of the Chemical Society, Perkin Transactions 1: Organic and Bio-Organic Chemistry (1998), (10), 1705-1713) in acetonitrile (10 ml) was heated to 90° C. for 22 h, then cooled and placed on an aminopropyl-SPE cartridge (10 g), eluting with methanol to give the crude product as a yellow solid (0.052 g) containing starting material. The solid was suspended in acetonitrile (5 ml), treated with more 3-amino-5-fluoropyridine dihydrochloride (0.057 g), and the mixture was heated to 90° C. for 18 h. The mixture was concentrated in vacuo to give the title compound as a brown solid (0.050 g).

LC/MS Rₜ 2.89 min m/z 362 [MH⁺].

### Intermediate 72. 4-Chloro-8-fluoro-6-(methylthio)-3-cinnolinecarboxamide

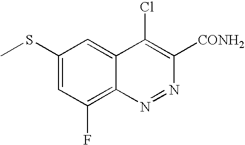

Tributyl(methylthio)stannane (0.124 g) (for example as prepared for Intermediate 44) was placed in a microwave vessel and a solution of 4-chloro-8-fluoro-6-iodo-3-cinnolinecarboxamide (0.13 g) (for example as prepared for Intermediate 69) in N,N-dimethylformamide (1.5 ml) was added followed by tetrakistriphenylphosphine palladium(0) (0.043 g). The mixture was heated under microwave irradiation for 10 mins at 130° C. The mixture was diluted with dichloromethane (10 ml), washed with water (10 ml), and the organic layer separated by hydrophobic frit. The aqueous layer was extracted with dichloromethane and the organic layers combined and evaporated. The crude product was taken up in dichloromethane, and purified by flash chromatography on silica gel (20 g silica cartridge), eluting with a gradient of 0-100% ethyl acetate in dichloromethane to give the title compound as a yellow-brown solid (0.037 g). Solid product remained on the cartridge frit and was collected and dried to give further title compound as a brown solid (0.019 g).

LC/MS Rₜ 2.44 min m/z 272 [MH⁺].

### Intermediate 73. 8-Fluoro-4-[(5-fluoro-3-pyridinyl)amino]-6-(methylthio)-3-cinnolinecarboxamide

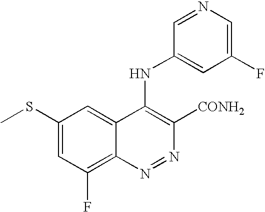

To a suspension of 4-chloro-8-fluoro-6-(methylthio)-3-cinnolinecarboxamide (0.055 g) (for example as prepared for Intermediate 72) in acetonitrile (5 ml) was added 3-amino-5-fluoropyridine dihydrochloride (0.041 g, Matrix Scientific; see also Journal of the Chemical Society, Perkin Transactions 1: Organic and Bio-Organic Chemistry (1998), (10), 1705-1713) and the suspension heated at 90° C. for 18 h. Starting material was present in the reaction mixture and a further portion of 3-amino-5-fluoropyridine dihydrochloride (9 mg; 0.25 equivalents) was added. Heating continued for 4 h. The cooled mixture was filtered and the solid washed with acetonitrile then dried under vacuum to give a yellow-brown solid (45 mg). The crude product was suspended in 1:1 dichloromethane/methanol, loaded onto an aminopropyl SPE cartridge (5 g), and eluted with 1:1 dichloromethane/methanol (50 ml) followed by methanol (20 ml). Solid remained on the surface of the cartridge which was dissolved by addition of triethylamine (3drops) in methanol (5 ml) followed by N,N-dimethylformamide (1 ml). The cartridge was further eluted with methanol, and all the fractions combined and evaporate to give the title compound as a cream solid (0.38 g).

LC/MS Rₜ 2.73 min m/z 348 [MH⁺].

### Intermediate 74. 4-Chloro-6-[(1,1-dimethylethyl)thio]-8-methyl-3-cinnolinecarboxamide

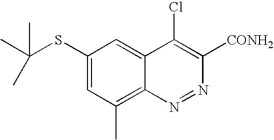

To 4-chloro-6-iodo-8-methyl-3-cinnolinecarboxamide (1.04 g) (for example as prepared for Intermediate 6) in a microwave vessel was added tributyl[(1,1-dimethylethyl)thio]stannane (1.36 g) followed by N,N-dimethylformamide (15 ml) and tetrakistriphenylphosphine palladium (0) (0.16 g), and the mixture was heated under microwave irradiation at 130° C. for 20 mins. The mixture was evaporated to dryness in vacuo and the residue triturated with hexane/ethyl acetate. The solid was filtered off, washed with hexane and diethyl ether then dried to give the title compound (0.62 g).

LC/MS Rₜ 3.28 min m/z 310 [MH⁺].

### Intermediate 78. 4-[(1-Ethyl-1H-pyrazol-5-yl)amino]-8-methyl-6-(Propylthio)-3-cinnolinecarboxamide

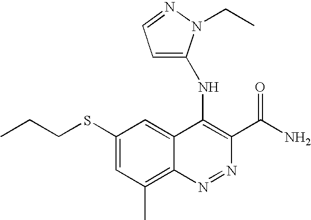

A suspension of 4-[(1-ethyl-1H-pyrazol-5-yl)amino]-6-iodo-8-methyl-3-cinnolinecarboxamide (100 mg) (for example as prepared for Intermediate 9), tributyl(propylthio)stannane (96 mg) (for example as prepared for Intermediate 59) and tetrakis(triphenylphosphine)palladium(0) (13 mg) in DMF was heated under microwave irradiation at 130° C. for 10 min. The mixture was blown to dryness under a stream of nitrogen and the residue taken up in 1:1 DMSO:MeOH and purified by mass directed preparative HPLC. The desired product was blown to dryness under a stream of nitrogen to give the title compound as a pale yellow solid (49 mg).

LC/MS Rt 3.2 min m/z 371 [MH⁺]

### Intermediate 79. 6-[(1,1-Dimethylethyl)thio]-4-[(5-fluoro-3-pyridinyl)amino]-8-methyl-3-cinnolinecarboxamide

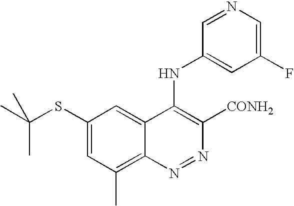

To 4-chloro-6-[(1,1-dimethylethyl)thio]-8-methyl-3-cinnolinecarboxamide (0.356 g) (for example as prepared for Intermediate 74) in acetonitrile (30 ml) was added 3-amino-5-fluoropyridine dihydrochloride (0.255 g, available from Matrix Scientific; see also Journal of the Chemical Society, Perkin Transactions 1: Organic and Bio-Organic Chemistry (1998), (10), 1705-1713) and the mixture stirred at reflux for 18 h. The mixture was evaporated to a small volume and the solid filtered off and washed with a small volume of acetonitrile. The solid was stirred vigorously in ethyl acetate and saturated sodium bicarbonate solution for 15 mins. The insoluble material was collected by filtration and washed with water then diethyl ether. The filtrates were combined, transferred to a separating funnel and the organic layer collected, washed with water, dried (MgSO₄) and evaporated. The residue was combined with the insoluble material and purified by flash chromatography on silica gel, eluting with a gradient of dichloromethane and methanol (100% CH₂Cl₂ to 3:1 CH₂Cl₂: MeOH) to give the title compound (0.389 g).

LC/MS Rₜ 3.26 min m/z 386 [MH⁺].

### Example 1

### 4-[(3-Cyanophenyl)amino]-8-methyl-6-(methylsulfonyl)-3-cinnolinecarboxamide

Method A

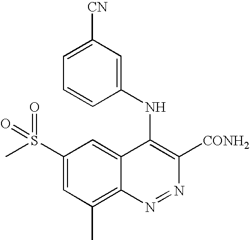

To a solution of 4-[(3-cyanophenyl)amino]-8-methyl-6-(methylthio)-3-cinnolinecarboxamide (0.12 g) (for example as prepared for Intermediate 8) in N,N′-dimethylformamide (5 ml) was added oxone® (available from Aldrich; 0.529 g) and the mixture stirred at room temperature for 2 h. The mixture was quenched by the addition of 10% sodium sulphite solution (10 ml) and extracted with chloroform (50 ml). The layers were separated by hydrophobic frit and the organic layer concentrated in vacuo. The residue was dissolved in 1:1 DMSO/MeOH (200 ml) and purified by mass directed preparative HPLC to give the title compound as a yellow solid (0.022 g).

LC/MS Rₜ 2.69 min m/z 382 [MH⁺].

1H NMR (400 MHz, DMSO-d6) δ ppm 11.64 (s, 1H), 8.94 (s, 1H), 8.16 (s, 2H), 7.93 (s, 1H), 7.78 (s, 1H), 7.67 (d, J=7.0 Hz, 1H), 7.51-7.60 (m, 2H), 3.15 (s, 3H), 2.97 (s, 3H)

The hydrophobic frit contained solid product suspended in the aqueous layer, which was collected by filtration to give a further quantity of the title compound as a yellow solid (0.035 g).

LC/MS Rₜ 2.6 min m/z 382 [MH⁺].

### Example 1

### 4-[(3-Cyanophenyl)amino]-8-methyl-6-(methylsulfonyl)-3-cinnolinecarboxamide

Method B

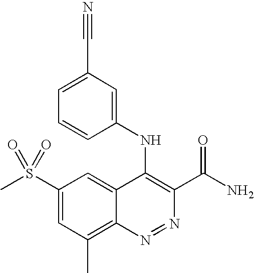

To 4-[(3-cyanophenyl)amino]-8-methyl-6-(methylthio)-3-cinnolinecarboxamide (27.4 g) (for example as prepared for Intermediate 8) in dry DMF (800 ml), mechanically stirred, was added oxone® (available from Aldrich; 96.4 g). The reaction was stirred at room temperature and monitored by hplc. After three and a half hours the mixture was cooled in an ice bath and aqueous sodium sulphite (29 g sodium sulphite in 500 ml water) was added. The orange colour became yellow and the suspension thickened to a precipitate; DMF (200 ml) was added during the addition and water (500 ml) was also added. This thick mixture was stirred for one hour and twenty minutes, filtered under vacuum and the solid washed well with water and sucked dry. The solid was then re-suspended in a mixture of DMSO (150 ml) and water (1000 ml), stirred well for 25 minutes and re-filtered under vacuum. The solid was washed with water, sucked as dry as possible, re-suspended in 5% DMSO in water (ca. 2l), and stirred well; the suspension was then filtered under vacuum and the residue washed with water (ca. 2l). The resulting yellow solid was dried at 45° C. under high vacuum to give the title compound (25.6 g; 85%), 97% pure by hplc.

LC/MS Rₜ 2.57 min m/z 382 [MH⁺].

### Example 1

### 4-[(3-Cyanophenyl)amino]-8-methyl-6-(methylsulfonyl)-3-cinnolinecarboxamide

Method C

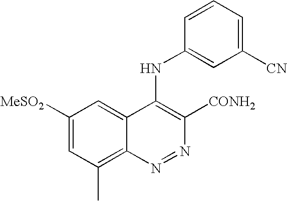

A stirred mixture of 4-[(3-cyanophenyl)amino]-6-iodo-8-methyl-3-cinnolinecarboxamide (13.5 g 0.031 mol) (for example as prepared for Intermediate 7) methanesulfinic acid sodium salt (available from Aldrich; 4.5 g, 0.044 mol), copper (I) iodide (available from Aldrich; 0.594 g, 0.003 mol) and 1,2-diaminocyclohexane (available from Aldrich; 0.783 ml, 0.0063 mol) in anhydrous DMSO (70 ml) was heated to 120° C. under nitrogen for 4 h. The reaction (complete by HPLC) was cooled to 80° C., and treated with Smopex-111® (0.81 g) for 30 min with stirring at 80° C. The suspension was cooled to 40° C., filtered through Celite® and washed with fresh DMSO (50 ml). The combined brown filtrates were warmed to 80° C., and treated slowly with 2:1 water:isopropanol (70 ml), causing a thick precipitate. The mixture was cooled slowly to room temperature overnight, filtered under reduced pressure, and the filtered solid washed with 1:1 DMSO:water (70 ml), 2:1 water:isopropanol (70 ml) and water (150 ml). The solid was dried in vacuo at 60° C. over P-₂O₅ to give the product as a yellow solid (10.4 g, 87%, purity 96% by area). The product (9.4 g) was dissolved in hot DMSO (150 ml) (solution not completely clear) and treated with isopropanol (150 ml), resulting in precipitation of a solid. After cooling to room temperature, the solid was filtered under reduced pressure, washed with isopropanol and then ether, and dried in vacuo at 40° C. to give the product as a pale yellow solid (7.5 g, 80%). A portion (3.8 g) of this solid was further purified by stirring in DMSO (380 ml) at 120° C. for 1.5 h, allowing to cool to room temperature overnight, heating again at 120° C. for 2 h, cooling to 80° C., and then filtering the suspension through a bed of Celite. The Celite pad was sucked dry, and water (740 ml) was added to the filtrate giving a yellow precipitate. The precipitate was filtered off, and dried to give the title compound as a yellow solid (2.53 g).

LC/MS Rₜ 2.6 min m/z 382 [MH⁺].

The celite pad was washed with hot DMSO (150 ml), and water (300 ml) added to the DMSO washings giving further yellow precipitate, which was filtered off and dried to give a second crop of the title compound (0.83 g).

LC/MS Rₜ 2.6 min m/z 382 [MH⁺].

Similarly prepared to Example 1 (Method A) were the following:

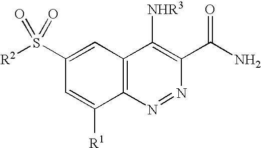

| | | | | Starting material- | | |
|---|---|---|---|---|---|---|
| | | | | identified as the | | |
| | | | | Chemical Structure as | | |
| Example | | | | depicted by the | | |
| No./salt | | | | Intermediate Number | | |
| form/ | | | | (for example as | | LCMS |
| isolation | | | | prepared by the method | LCMS | Rₜ |
| method | R¹ | R² | R³NH— | described) | MH⁺ | (min) |
| 2(a) | Me | tBu | 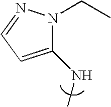 | 6-[(1,1-Dimethylethyl)thio]-4-[(1-ethyl-1H-pyrazol-5-yl)amino]-8-methyl-3-cinnolinecarboxamide(for example asprepared forIntermediate 10) | 417 | 2.67 |
| 4(a) | H | Me | 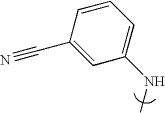 | 4-[(3-Cyanophenyl)amino]-6-(methylthio)-3-cinnolinecarboxamide(assumed to be thehydrochloride) (forexample as prepared forIntermediate 30) | 368 | 2.46 |
| 5(b) | H | Me | 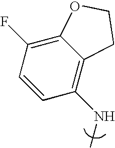 | (for example asprepared forIntermediate 31) | 403 | 2.60 |
| 7(b) | H | Me | 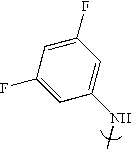 | (for example asprepared forIntermediate 76) | 379 | 2.69 |
| 8(b) | H | Me | 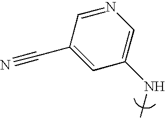 | (for example asprepared forIntermediate 32) | 369 | 2.18 |
| 9(b) | H | Me | 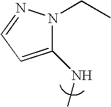 | 4-[(1-Ethyl-1H-pyrazol-5-yl)amino]-6-(methylthio)-3-cinnolinecarboxamide(for example asprepared forIntermediate 33) | 361 | 2.21 |
| 10(c) | H | Me | 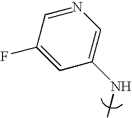 | (for example asprepared forIntermediate 34) | 362 | 2.22 |
| 11(b) | H | Et | 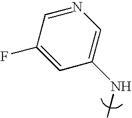 | (for example asprepared forIntermediate 35) | 376 | 2.34 |
| 12(b) | H | tBu | 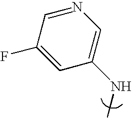 | (for example asprepared forIntermediate 36) | 404 | 2.67 |
| 14(b) | H | Et | 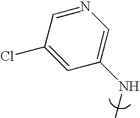 | (for example asprepared forIntermediate 38) | 392/394 | 2.48 |
| 15(b) | H | Et | 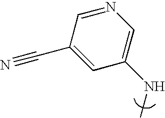 | (for example asprepared forIntermediate 39) | 383 | 2.32 |
| 16(d) | H | Et | 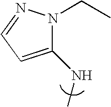 | (for example asprepared forIntermediate 40) | 375 | 2.32 |
| 17(e) | Me | Me | 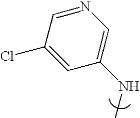 | 4-[(5-Chloro-3-pyridinyl)amino]-8-methyl-6-(methylthio)-3-cinnolinecarboxamide(for example asprepared forIntermediate 15) | 392 | 2.51 |
| 18(e) | Me | Me | 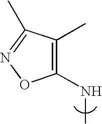 | 4-[(3,4-Dimethyl-5-isoxazolyl)amino]-8-methyl-6-(methylthio)-3-cinnolinecarboxamide(for example asprepared forIntermediate 16) | 376 | 2.47 |
| 19(d) | H | tBu | 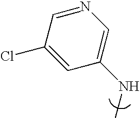 | (for example asprepared forIntermediate 41) | 420 | 2.67 |
| 20(b) | H | tBu | 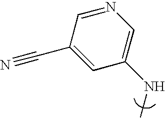 | (for example asprepared forIntermediate 42) | 411 | 2.53 |
| 21(d) | H | tBu | 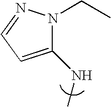 | (for example asprepared forIntermediate 43) | 403 | 2.49 |
| 22(f) | Me | Me | 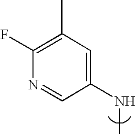 | 4-[(6-Fluoro-5-methyl-3-pyridinyl)amino]-8-methyl-6-(methylthio)-3-cinnolinecarboxamideor example as preparedfor Intermediate 17) | 390 | 2.54 |
| 23(g) | Me | Et | 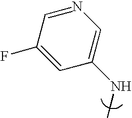 | (for example asprepared forIntermediate 18) | 390 | 2.47 |
| 24(a) | Me | Et | 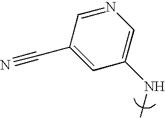 | (for example asprepared forIntermediate 77) | 397 | 2.44 |
| 33(a) | Me | n-Pr | 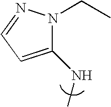 | 4-[(1-Ethyl-1H-pyrazol-5-yl)amino]-8-methyl-6-(propylthio)-3-cinnolinecarboxamide(for example asprepared forIntermediate 78) | 403 | 2.59 |

(a) Product isolated by extraction with CH₂Cl₂ followed by mass directed preparative HPLC
(b) Filtration from the reaction mixture after quenching with sodium sulphite and diluting with water.
(c) Isolation by extraction with CH₂Cl₂/water, followed by chromatography on silica gel (eluting with CH₂Cl₂/methanol/Et₃N), followed by mass directed preparative HPLC
(d) Isolation by extraction with CH₂Cl₂/water, followed by chromatography on silica gel (eluting with CH₂Cl₂/methanol/Et₃N).
(e) Product isolated by extraction with CH₂Cl₂ followed by trituration with ether/dichloromethane, followed by mass directed preparative HPLC
(f) Isolation by extraction with CH₂Cl₂/water, filtering off of precipitated product, followed by chromatography of the pre-adsorbed (Florisil) precipitate on silica gel (eluting with CH₂Cl₂/methanol/Et₃N).
(g) Product isolated by extraction with CH₂Cl₂ followed by trituration with ether, followed by mass directed preparative HPLC

### Example 3

### 4-[(1-Ethyl-1H-pyrazol-5-yl)amino]-6-(ethylsulfonyl)-8-methyl-3-cinnolinecarboxamide

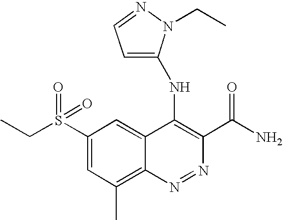

To a solution of 4-[(1-ethyl-1H-pyrazol-5-yl)amino]-6-(ethylthio)-8-methyl-3-cinnolinecarboxamide (54 mg) (for example as prepared for Intermediate 11) in DMF (2 ml) was added oxone (187 mg) and the mixture stirred at 20° C. for 2 h. The mixture was quenched with 10% sodium sulphite solution (20 ml) and extracted with dichloromethane (20 ml). The organic layers were separated by hydrophobic frit and blown to dryness under a stream of nitrogen. The crude product was purified by mass directed preparative HPLC to give a yellow solid (9 mg). This solid was combined with further material (10 mg) from another similar experiment, and further purified by eluting with methanol through an aminopropyl SPE cartridge (1 g); the eluent was evaporated to give an orange solid (14 mg). This solid was applied in DMSO/methanol to an aminopropyl SPE cartridge (2 g) and eluted with methanol. Evaporation of the eluent gave an orange solid (12 mg). This solid was dissolved in dichloromethane: methanol (ca. 2:1, 5 ml) and shaken with MP-carbonate resin (Argonaut) (100 mg) for 5 h. The resin was filtered off and the solvent evaporated to give the title compound (8 mg).

LC/MS Rₜ 2.46 min m/z 389 [MH⁺].

### Example 6

### 4-[(3-Chloro-2-fluorophenyl)amino]-6-(methylsulfonyl)-3-cinnolinecarboxamide

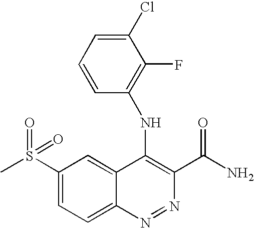

Oxone (81 mg) was added to a stirred solution of 4-[(3-chloro-2-fluorophenyl)amino]-6-(methylthio)-3-cinnolinecarboxamide (19 mg) (for example as prepared for Intermediate 75) in DMF (2 ml) and the mixture was stirred at room temperature for 3.5 h. More oxone (40 mg) was added and stirring was continued for 2 h. Saturated aqueous sodium sulphite (2 ml) was added, and the mixture stirred for 5 min. Water (10 ml) was then added, giving a precipitate which was filtered off, washed with water and dried to give a pale yellow solid (14 mg). This solid was dissolved in DMSO (0.5 ml) and purified by mass directed preparative HPLC; the product fraction was diluted with acetonitrile and stirred with MP-carbonate resin (Argonaut, 90 mg) for 1.5 h. The resin was filtered off, washed with acetonitrile and the filtrate evaporated in vacuo to give the title compound as a pale yellow solid (8.2 mg).

LC/MS Rₜ 2.79 min m/z 395/397 [MH⁺].

### Example 13

### 4-[(5-Chloro-3-pyridinyl)amino]-6-(methylsulfonyl)-3-cinnolinecarboxamide

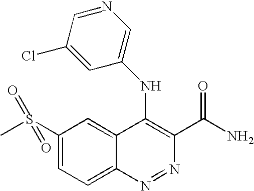

4-[(5-Chloro-3-pyridinyl)amino]-6-(methylthio)-3-cinnolinecarboxamide (60 mg) (for example as prepared for Intermediate 37) was stirred at room temperature under nitrogen with oxone (267 mg) in dry DMF (5 ml) for 4 h. The mixture was quenched with saturated aqueous sodium sulphite (6 ml), stirred for 5 min at room temperature, and diluted with water (25 ml). The resulting precipitate was washed with water and dried to give a pale yellow solid (49 mg). This solid was suspended in DMSO (1 ml) and methanol (4 ml), and the solid filtered off, washed with methanol and dried to give a pale yellow solid (44 mg).

This solid was suspended in 50/50 dichloromethane/methanol and pre-adsorbed onto Florisil. Purification by flash chromatography on silica gel (10 g column), eluting with a solvent gradient containing 0-15% methanol and 0-0.15% triethylamine in dichloromethane over 20 min, gave the title compound as a yellow solid (20.4 mg).

LC/MS Rₜ 2.39 min m/z 378/380 [MH⁺].

### Example 25

### 4-{[4-Fluoro-3-(methyloxy)phenyl]amino}-8-methyl-6-(methylsulfonyl)-3-cinnolinecarboxamide

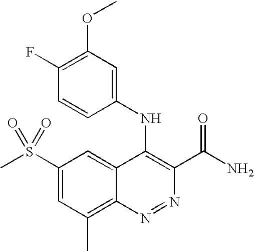

To 4-chloro-8-methyl-6-(methylsulfonyl)-3-cinnolinecarboxamide (40 mg) (for example as prepared for Intermediate 14) in acetonitrile (3 ml) was added 4-fluoro-3-(methyloxy)aniline (19 mg, available from Fluorochem) and the mixture heated under nitrogen at 80° C. over the weekend, then allowed to cool to room temperature. The mixture was filtered and filtrate concentrated in vacuo. The residue was purified mass directed preparative HPLC to give the title compound as a pale yellow solid (12 mg).

LC/MS Rₜ 2.76 min m/z 405 [MH⁺].

### Example 26

### 4-[(5-Fluoro-3-pyridinyl)amino]-8-methyl-6-(methylsulfonyl)-3-cinnolinecarboxamide

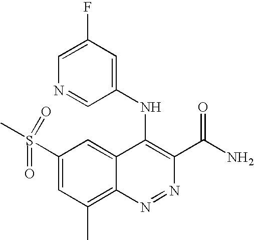

To 4-chloro-8-methyl-6-(methylsulfonyl)-3-cinnolinecarboxamide (150 mg) (for example as prepared for Intermediate 14) in acetonitrile (5 ml) was added pyridine hydrochloride (86 mg, available from Aldrich) and 5-fluoro-3-pyridinamine dihydrochloride (108 mg, Matrix Scientific; see also Journal of the Chemical Society, Perkin Transactions 1: Organic and Bio-Organic Chemistry (1998), (10), 1705-1713) and the mixture heated at 80° C. until no starting material was observed by LCMS, before being cooled to room temperature and the solvent blown off. The residue was purified by mass directed preparative HPLC to give the title compound as an off white solid (17 mg).

LC/MS Rₜ 2.37 min m/z 376 [MH⁺].

Similarly prepared from 4-chloro-8-methyl-6-(methylsulfonyl)-3-cinnolinecarboxamide (Intermediate 14) were the following:

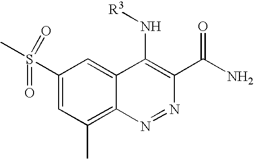

| Example | | | LC/MS | LC/MS |
|---|---|---|---|---|
| Number | R³NH— | Amine Reagent/Source | Rₜ (min) | MH⁺ |
| 27 | 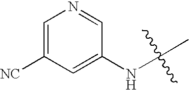 | 5-Amino-3-pyridinecarbonitrile/ChemPacific Product List/Journal of MedicinalChemistry (1967), 10(2),149-54 | 2.34 | 383 |
| 28(a) | 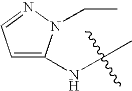 | 1-Ethyl-1H-pyrazol-5-amine/Aldrich | 2.37 | 375 |

(a) A second purification by mass directed preparative HPLC (30 min gradient method) was required.

### Example 29

### 4-[(2,3-Difluorophenyl)amino]-8-methyl-6-(methylsulfonyl)-3-cinnolinecarboxamide

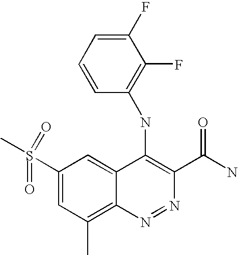

To 4-chloro-8-methyl-6-(methylsulfonyl)-3-cinnolinecarboxamide (150 mg) (for example as prepared for Intermediate 14) in acetonitrile (8 ml) was added 2,3-difluoroaniline (65 mg, available from Aldrich) and the mixture heated at 80° C. for 3 hours. The solvent was removed in vacuo, the residue triturated with diethyl ether (˜10 ml) and the precipitate collected by filtration. The solid was purified by mass directed preparative HPLC to give the title compound as yellow solid (12 mg).

LC/MS Rₜ 2.74 min m/z 393 [MH⁺].

### Example 30

### 4-[(3-Chloro-2-fluorophenyl)amino]-8-methyl-6-(methylsulfonyl)-3-cinnolinecarboxamide

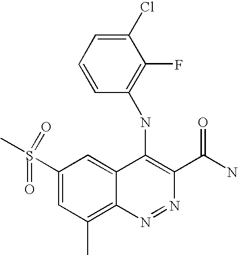

To 4-chloro-8-methyl-6-(methylsulfonyl)-3-cinnolinecarboxamide (120 mg) (for example as prepared for Intermediate 14) in acetonitrile (5 ml) was added 3-chloro-2-fluoroaniline (58 mg, available from Aldrich) and the mixture heated at 80° C. overnight. The mixture was cooled to room temperature and the solvent blown off under a stream of nitrogen. The residue was triturated with diethyl ether and the precipitate collected by filtration. The solid was purified by mass directed preparative HPLC; fractions containing the desired mass were combined and the solvent blown off with nitrogen at 40° C. to give a yellow solid. The solid was loaded onto a 10 g sulphonic acid ion exchange cartridge (Isolute, SCX). The cartridge was washed with methanol and then eluted with 2M ammonia in methanol and the solvent blown down. The residue was purified by mass directed preparative HPLC to give the title compound as a yellow solid (20 mg).

LC/MS Rₜ 2.86 min m/z 409 [MH⁺].

### Example 31

### 4-[(7-Fluoro-2,3-dihydro-1-benzofuran-4-yl)amino]-8-methyl-6-(methylsulfonyl)-3-cinnolinecarboxamide

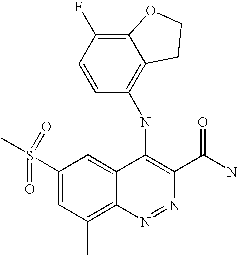

To 4-chloro-8-methyl-6-(methylsulfonyl)-3-cinnolinecarboxamide (100 mg) (for example as prepared for Intermediate 14) in acetonitrile (5 ml) was added 7-fluoro-2,3-dihydro-1-benzofuran-4-amine 50 mg (for example as prepared for Intermediate 50) and the mixture heated at 80° C. for 6 hours. The mixture was cooled to room temperature and the solvent blown off. The residue was purified by mass directed preparative HPLC. The residue was triturated with diethyl ether and the precipitate collected to give the title compound as a yellow solid (24 mg).

LC/MS Rₜ 2.75 min m/z 417 [MH⁺].

### Example 32

### 4-[(3,5-Difluorophenyl)amino]-8-methyl-6-(methylsulfonyl)-3-cinnolinecarboxamide

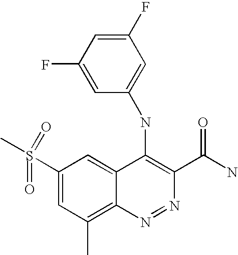

To 4-chloro-8-methyl-6-(methylsulfonyl)-3-cinnolinecarboxamide (150 mg) (for example as prepared for Intermediate 14) in acetonitrile (8 ml) was added 3,5-difluoroaniline (65 mg, available from Aldrich) and the mixture heated at 80° C. for 90 minutes. The reaction was cooled to room temperature and the solvent removed. The residue was triturated with diethyl ether and the resultant precipitate collected by filtration. The solid was purified by mass directed preparative HPLC to give the title compound as a yellow solid (15 mg).

LC/MS Rₜ 2.79 min m/z 393 [MH⁺].

### Example 34

### 6-[(1,1-Dimethylethyl)sulfonyl]-4-[(5-fluoro-3-pyridinyl)amino]-8-methyl-3-cinnolinecarboxamide

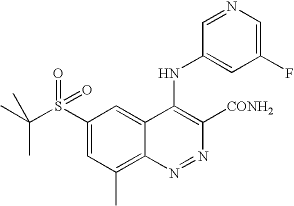

To 6-[(1,1-dimethylethyl)thio]-4-[(5-fluoro-3-pyridinyl)amino]-8-methyl-3-cinnolinecarboxamide (0.385 g) (for example as prepared for Intermediate 79)) in N,N-dimethylformamide (30 ml) was added oxone (1.23 g) and the mixture stirred at room temperature for 18 h. Saturated sodium sulphite solution was added with vigorous stirring. A precipitate which formed was collected by filtration, and washed well with water followed by a little dichloromethane and finally diethyl ether to give the title compound (0.242 g).

LC/MS Rₜ 2.69 min m/z 418 [MH⁺].

### Example 35

### 4-[(5-Chloro-3-pyridinyl)amino]-6-(ethylsulfonyl)-8-methyl-3-cinnolinecarboxamide

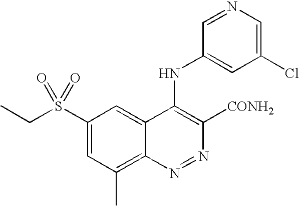

A solution of 4-[(5-chloro-3-pyridinyl)amino]-6-(ethylthio)-8-methyl-3-cinnolinecarboxamide hydrochloride 0.09 g) (for example as prepared for Intermediate 61) in N,N-dimethylformamide (5 ml) was placed on an aminopropyl SPE cartridge (1 g) and eluted with N,N-dimethylformamide (5 ml). The solution was stirred and treated with oxone (0.271 g) for 2 h. The mixture was quenched by use of 10% sodium sulphite solution (20 ml) and extracted with dichloromethane (20 ml). The organic layer was separated by hydrophobic frit and blown to dryness under a stream of nitrogen to give the title compound as a pale yellow solid (0.059 g; 66%).

LC/MS Rₜ 2.61 min m/z 406 [MH⁺].

### Example 36

### 6-(Ethylsulfonyl)-8-fluoro-4-[(5-fluoro-3-pyridinyl)amino]-3-cinnolinecarboxamide

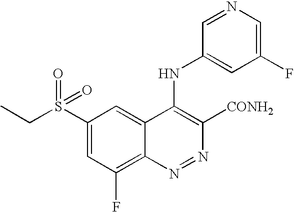

A solution of 6-(ethylthio)-8-fluoro-4-[(5-fluoro-3-pyridinyl)amino]-3-cinnolinecarboxamide (0.05 g) (for example as prepared for Intermediate 71) in N,N-dimethylformamide (2 ml) was placed on an aminopropyl SPE cartridge (2 g) and eluted with N,N-dimethylformamide (˜4 ml). The solution obtained was treated with oxone (0.17 g) and stirred at room temperature for 22 h. A further portion of oxone (0.17 g) was added and stirring continued for 22 h. More oxone (0.17 g) was added and the mixture stirred for 8 h, quenched using 10% sodium sulphite solution (20 ml), and extracted with dichloromethane (20 ml). The organic layer was separated by hydrophobic frit, concentrated in vacuo and the residue purified by mass directed preparative HPLC to give the title compound as a pale yellow solid (0.023 g; 43%).

LC/MS Rₜ 2.36 min m/z 394 [MH⁺].

### Example 37

### 8-Fluoro-4-[(5-fluoro-3-pyridinyl)amino]-6-(methylsulfonyl)-3-cinnolinecarboxamide

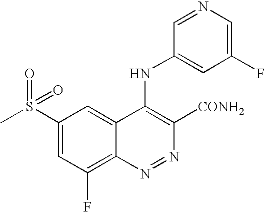

To a suspension of 8-fluoro-4-[(5-fluoro-3-pyridinyl)amino]-6-(methylthio)-3-cinnolinecarboxamide (37 mg) (for example as prepared for Intermediate 73) in dry N,N-dimethylformamide (2 ml) was added oxone (0.131 g) and the mixture stirred at room temperature for 45 mins. Saturated sodium sulphite (15 ml) was added and stirring continued for 20 mins. The dense yellow precipitate was collected by filtration, washed with water (×3), dried under vacuum and then washed with diethyl ether. The solid was collected from the filter tube by slurrying with dichloromethane and blowing to dryness under a stream of nitrogen to give a yellow solid (0.0204 g) which contained intermediate sulphoxide. This solid was suspended in N,N-dimethylformamide (2 ml), oxone (0.02 g) added, and the suspension stirred at room temperature for 2 h. A further portion of oxone (0.01 g) was added, and stirring was continued for 1.5 h. The mixture was quenched by addition of saturated sodium sulphite (10 ml) and stirring was continued for 15 mins. The mixture was filtered, and the residue washed with water followed by diethyl ether and dried under vacuum. The crude product was suspended in N,N-dimethylformamide (4 ml), more oxone (0.045 g) added, and the mixture stirred for 2 h at room temperature then left to stand at room temperature overnight. The mixture was quenched by addition of saturated sodium sulphite, stirred for 15 mins and extracted with dichloromethane (2×10 ml). The organic layer was separated by hydrophobic frit and evaporated to give the title compound as a yellow solid (6.5 mg).

LC/MS Rₜ 2.25 min m/z 380 [MH⁺].

### Example 38

### 4-[(1-Ethyl-1H-pyrazol-5-yl)amino]-6-(ethylsulfonyl)-8-methyl-3-cinnolinecarboxamide

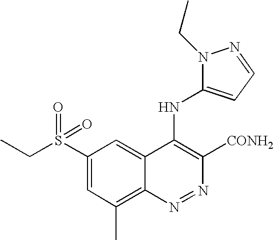

To 4-[(1-ethyl-1H-pyrazol-5-yl)amino]-6-(ethylthio)-8-methyl-3-cinnolinecarboxamide (427 mg 1.2 mmol) (for example as prepared for Intermediate 11) in N,N-dimethylformamide (50 ml) was added oxone (1.48 g; 2.4 mM) and the mixture was stirred at room temperature over the weekend. Saturated sodium sulphite solution was added and the solid collected by filtration, washed thoroughly with water followed by diethyl ether, and dried in vacuo to give the title compound (0.275 g).

LC/MS Rₜ 2.47 min m/z 389 [MH⁺].

Example 1 may also be prepared according to the following scheme:

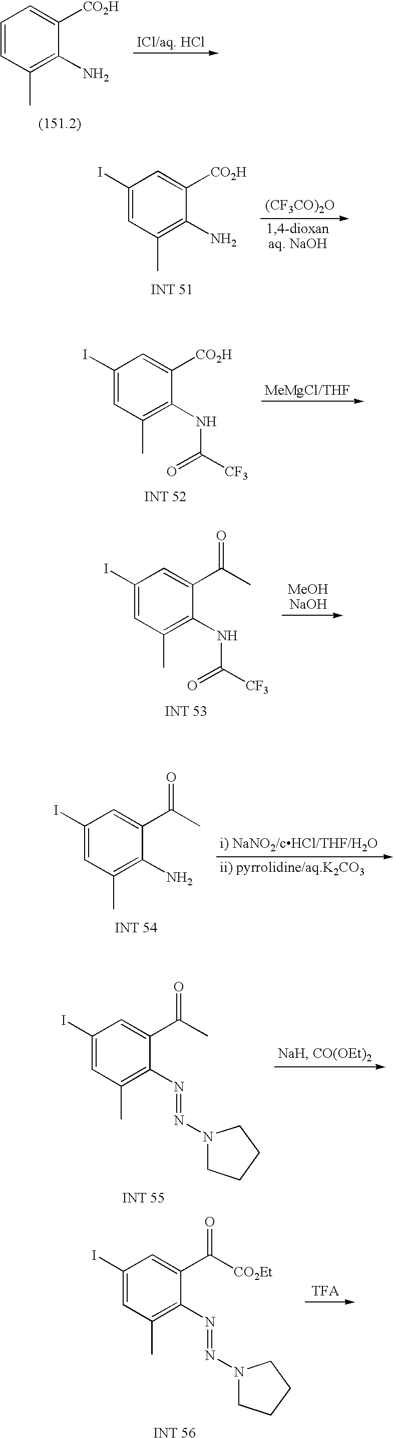

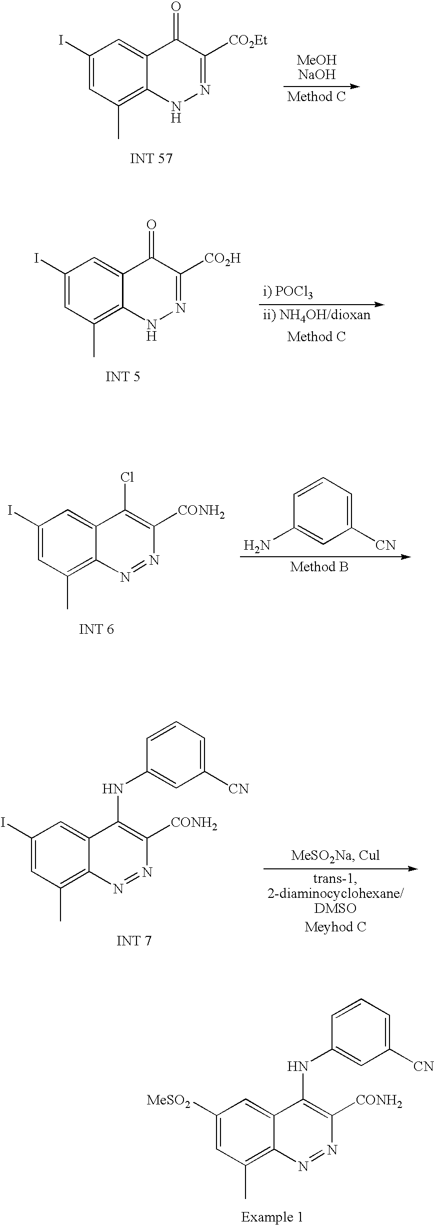

The following is an adaptation of the previous route and may be suitable (e.g.) for scaling up

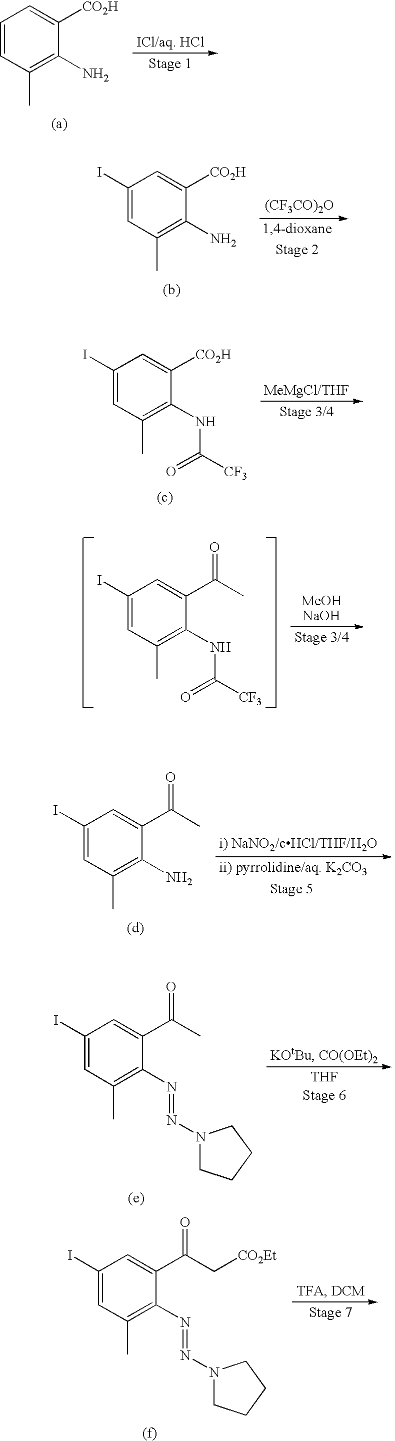

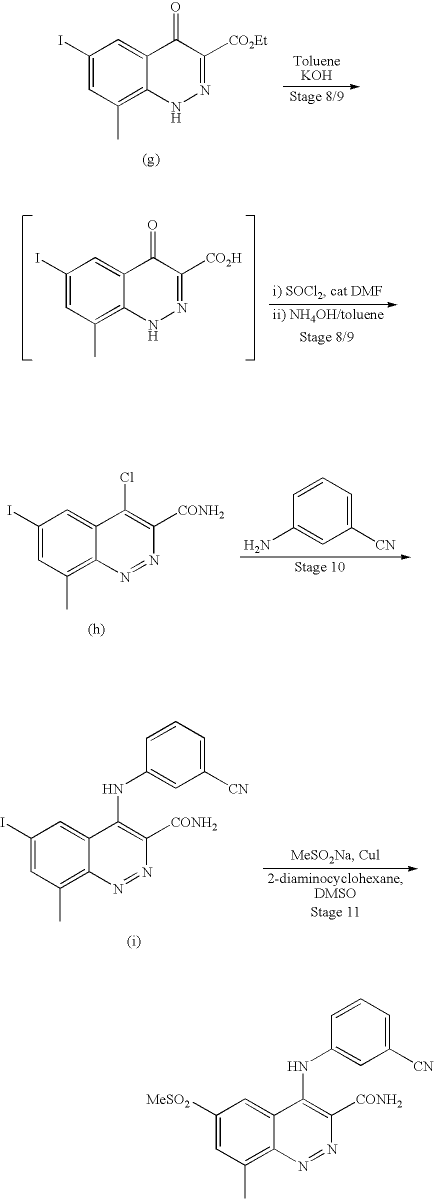

Stage 1—Intermediate (b)

A solution of anthracilicacid (200 g dissolved in HCl) is added to a solution of iodine monochloride (257.6 g dissolved in HCl) and stirred vigorously at 0-10° C. for 30 minutes-2 hours. The temperature is raised to 25-35° C. and maintained for 4-6 hours. The mixture is filtered and washed 3 times with water (1400 ml×3) and dried in vacuo for 8-12 hours.

Stage 2—Intermediate (c)

Intermediate (c) (315 g) is added to 1890 ml 1,4 dioxane and stirred. The mix is cooled to 10-15° C. and 286.51 g trifluoro acetic acid is added dropwise over 30-40 minutes. This is stirred at 10-15° C. for 1½ to 2½ hours. The reaction mix is concentrated under vacuum below 70° C. and cooled to 35-45° C. 1575 ml water is added and the mixture stirred at 30-45° C. The temperature is decreased to 25-35° C. and the mixture stirred for 1-2 hours. The suspension is filtered and washed with water (630 ml) and cyclohexane (1260 ml and 472.5 ml) and dried in vacuo 8-12 hours.

Stage 3-4—Intermediate (d)

Intermediate (c) 100 g and 1500 ml THF under nitrogen is cooled to 0-5° C. MeMgCl (2.1 ml in THF 606.31 ml) is added dropwise over 2-4 hours. The reaction mixture is warmed to 10-15° C., thus held for 3 hours and then slowly added to 800 g crushed ice in 200 ml water 0-10° C. The pH is adjusted to 2-3 with the addition of 180 ml 33-35% HCl in 820 ml water. The reaction mixture is then extracted with ethyl acetate which is then washed with aq. sodium carbonate. The ethyl acetate phase is reduced to 1-2 volumes by distillation, then treated with iso-propanol (2vols). A solvent swap between ethyl acetate/iso-propanol and methanol is then carried out and the resulting methanol solution treated with aq. sodium hydroxide. The reaction mixture is heated to reflux, cooled to ambient, then treated with water.

The resulting suspension is stirred 30 min-1 hour, filtered and washed with water (500 ml×2). The wet cake is dried 60-70° C. for 10-15 hours.

Stage 5—Intermediate (e)

Intermediate (d) (100 g), 500 ml THF and 70 ml water are cooled to −5-0° C. 150 ml concentrated HCl (33-37%) is slowly added. Cooled sodium nitrite solution (30.1 g in 200 ml water) is slowly added and the mix stirred 15-45 minutes. Potassium carbonate solution (200 g in 1400 ml water) is added to 51.7 g pyrrolidine at 25-30° C. and the two solutions mixed at −5-0° C.

The mix is stirred 60-90 minutes at −10-0° C. The THF is distilled off under vacuum at 50-55° C., the reaction mass cooled to 25-30° C. and stirred for 30 minutes -1 hour at 25-35° C. The resulting suspension is filtered and washed with water, toluene and hexane. The product is harvested then dried in vacuo at 50-55° C. for 6-7 hours.

Stage 6—Intermediate (f)

165 g diethylcarbonate, 300 ml THF and 94.3 g potassium tertiary butoxide is heated to 70-75° C. under nitrogen. Intermediate (e) (100 g in 700 ml THF) is added and the mixture heated at reflux 1-2 hours. The reaction mixture is cooled and added to 70 g ammonium chloride in 700 ml water. The reaction mixture is stirred 10-15 minutes at 15-25° C. then extracted with ethyl acetate. The ethyl acetate extracts are washed with aq NaCl. A solvent swap between ethyl acetate and toluene is then carried out by successive distillations and additions of toluene. The toluene solution is then cooled to ambient temperature and treated with di-isopropyl ether and hexane to form a suspension. The suspension was cooled to 0-5° C., filtered, washed with di-isopropyl ether and hexane, harvested and dried in vacuo at 45-50° C. for 6-8 hours,

Stage 7—Intermediate (g)

79.9 g of trifluoroacetic acid and 30 ml DCM is cooled to 0-5° C. Intermediate (f) (100 g in 300 ml DCM) is added slowly and stirred 1-2 hours at 0-5° C. The reaction mass is distilled at 40-45° C., cooled to 25-35° C. and 500 ml water added over 15-20 minutes. The resulting suspension is stirred for 1-2 hours, filtered and washed with water (200 ml×2). The wet cake is dried 8-10 hours at 60-70° C.

Stages 7-9 Intermediate (h)

37.53 g KOH (in 200 ml H₂0) is added to 100 g intermediate (g) and 1500 ml toluene over a period of 10-15 minutes at 25-35° C. 1 g tetrabutyl ammonium iodide is added and the temperature raised to 110° C. for 1-2 hours, the solvent is distilled and the reaction mix cooled to 90-100° C. 1500 ml toluene is added and the solvent distilled. Thionyl chloride (830.49 g) and 30 ml dimethyl formamide is added and the temperature raised to 85-90° C. and thus held for 3-4 hours after which the mixture is to cooled to −5 to −10° C. and charged into 5000 ml aqueous ammonia over 1-2 hours.

The resulting suspension is stirred for 1-2 hours, filtered and washed with water (2000 ml×3). After drying in vacuo for 8-10 hours at 35-45° C., the crude product is resuspended in acetone (100 ml) and diisopropyl ether (400 ml ), stirred at 5-10° C. for 30-60 mins, then filtered and washed with diisopropyl ether.

The resultant product is dried 30-35° C. for 4-7 hours.

Stage 10—Intermediate (i)

25 g of Intermediate (h) in 750 ml acetonitrile is heated to 55-60° C. A solution of 9.4 g 3-aminobenzonitrile in 500 ml acetonitrile is added slowly over 2-3 hours at 55° C. The reaction mixture is maintained at 55-65° C. for 14-16 hours, analysed to confirm the reaction is complete then cooled to 25-35° C. The resultant suspension is filtered and then resuspended in 250 ml water. The suspension is filtered, then again resuspended in a pre-prepared solution of 15 ml triethylamine in 250 ml water and stirred for 15-20 minutes. The suspension is filtered, washed with 625 ml water or until such time as the pH of the washings are shown to 7-8. The product is collected and dried in vacuo at 60-70° C. for 8-10 hours.

Stage 11

5 g Intermediate (i), 25 ml DMSO and 2.4 g sodium methane sulphinate are heated with stirring to 130° C.-140° C. for 8-9 hours. The reaction mixture is cooled to 25-35° C. and 250 ml water added. The resulting suspension is then stirred for 20-30 minutes then filtered and washed with water. The crude product is suspended in 65 ml DMSO and the temperature increased to 60-70° C. 65 ml isopropyl alcohol is added dropwise to effect precipitation of the solid product. The temperature is then lowered to 35-40° C. and the suspension stirred for 20-30 minutes, filtered and washed with water (2×100 ml) and isopropyl alcohol (2×−50 ml and 30 ml). The product is dried for 8-10 hours at 60-70° C.

What is claimed is:

1. A compound of formula (I): 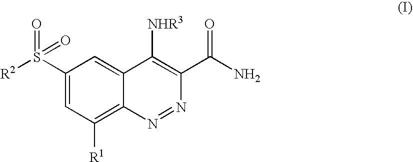
wherein:
R¹ is hydrogen, fluoro or methyl;
R² is C₁₋₆alkyl; and
R³ is phenyl or pyridinyl, each of which is unsubstituted or substituted with one or two substituents which may be the same or different selected from fluoro, chloro, cyano, methyl or methoxy; phenyl fused to a 5-membered saturated ring containing one oxygen atom and optionally substituted on the phenyl ring with one fluoro; 3,4 dimethyl isoxazolyl; or N—C₁₋₂alkyl-pyrazolyl; or a pharmaceutically acceptable salt or solvate thereof.

2. A compound according to claim 1 wherein R¹ is H or methyl.

3. A compound according to claim 1 wherein R² is methyl, ethyl, t-butyl or n-propyl.

4. A compound according to any one of claim 1 wherein R³ is 3-cyanophenyl, 2,3-difluorophenyl, 3,5-difluorophenyl, 2-fluoro-3-chlorophenyl, 4-fluoro-3-(methoxy)phenyl, 5-cyano-3-pyridinyl, 5-fluoro-3-pyridinyl, 5-chloro-3-pyridinyl, 7-fluoro-2,3-dihydro-1-benzofuran-4-yl, 1-ethyl-1H-pyrazol-5-yl, 3,4-dimethyl-5-isoxazolyl or 6-fluoro-5-methyl-3-pyridinyl.

5. A compound according to claim 4 wherein R³ is 3-cyanophenyl, 2,3-difluorophenyl, 3,5-difluorophenyl, 2-fluoro-3-chlorophenyl, 4-fluoro-3-(methoxy)phenyl, 5-cyano-3-pyridinyl, 5-fluoro-3-pyridinyl, 5-chloro-3-pyridinyl, 7-fluoro-2,3-dihydro-1-benzofuran-4-yl or 1-ethyl-1H-pyrazol-5-yl.

6. A compound according to claim 4 wherein R³ is 3-cyanophenyl, 3,5-difluorophenyl, 5-fluoro-3-pyridinyl, 1-ethyl-1H-pyrazol-5-yl, 5-chloro-3-pyridinyl, or 3,4-dimethyl-5-isoxazolyl.

7. A compound according to claim 6 wherein R³ is 3-cyanophenyl, 3,5-difluorophenyl, 5-fluoro-3-pyridinyl, 5-chloro-3-pyridinyl or 1-ethyl-1H-pyrazol-5-yl.

8. A compound according to claim 1 which is formula (Ia): 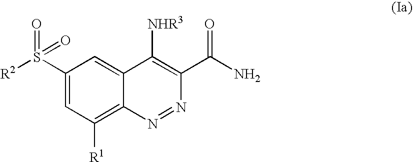
wherein:
R¹ is hydrogen, fluoro or methyl;
R² is C₁₋₆alkyl; and
R³ is selected from phenyl or pyridinyl each of which is unsubstituted or substituted with one substitutent selected from fluoro, chloro or cyano; phenyl substituted with two substituents which may be the same or different selected from fluoro, chloro or methoxy; phenyl fused to a 5-membered saturated ring containing one oxygen atom and optionally substituted on the phenyl ring with one fluoro; or N—C₁₋₂alkyl-pyrazolyl; or a pharmaceutically acceptable salt or solvate thereof.

9. A compound according to claim 8 in which R¹ is methyl.

10. A compound according to claim 8 in which R² is methyl.

11. A compound according to claim 8 in which R³ is 3-cyanophenyl.

12. A compound according to claim 1 selected from the group consisting of:
4-[(3-cyanophenyl)amino]-8-methyl-6-(methylsulfonyl)-3-cinnolinecarboxamide;
6-[(1,1-dimethylethyl)sulfonyl]-4-[( 1-ethyl-1H-pyrazol-5-yl)amino]-8-methyl-3-cinnolinecarboxamide;
4-[(1-ethyl-1H-pyrazol-5-yl )amino]-6-(ethylsulfonyl)-8-methyl-3-cinnolinecarboxamide;
4-[(3-cyanophenyl)amino]-6-(methylsulfonyl)-3-cinnolinecarboxamide;
4-[(7-fluoro-2,3-dihydro-1-benzofuran-4-yl)amino]-6-(methylsulfonyl)-3-cinnolinecarboxamide;
4-[(3-chloro-2-fluorophenyl)amino]-6-(methylsulfonyl)-3-cinnolinecarboxamide;
4-[(3,5-difluorophenyl)amino]-6-(methylsulfonyl)-3-cinnolinecarboxamide;
4-[(5-cyano-3-pyridinyl)amino]-6-(methylsulfonyl)-3-cinnolinecarboxamide;
4-[(1-ethyl-1H-pyrazol-5-yl)amino]-6-(methylsulfonyl)-3-cinnolinecarboxamide;
4-[(5-fluoro-3-pyridinyl)amino]-6-(methylsulfonyl)-3-cinnolinecarboxamide;
6-(ethylsulfonyl)-4-[(5-fluoro-3-pyridinyl)amino]-3-cinnolinecarboxamide;
6-[(1,1-dimethylethyl)sulfonyl]-4-[(5-fluoro-3-pyridinyl)amino]-3-cinnolinecarboxamide;
4-[(5-chloro-3-pyridinyl)amino]-6-(methylsulfonyl)-3-cinnolinecarboxamide;
4-[(5-chloro-3-pyridinyl)amino]-6-(ethylsulfonyl)-3-cinnolinecarboxamide;
4-[(5-cyano-3-pyridinyl)amino]-6-(ethylsulfonyl)-3-cinnolinecarboxamide;
4-[(1-ethyl-1H-pyrazol-5-yl)amino]-6-(ethylsulfonyl)-3-cinnolinecarboxamide;
4-[(5-chloro-3-pyridinyl)amino]-8-methyl-6-(methylsulfonyl)-3-cinnolinecarboxamide;
4-[(3,4-dimethyl-5-isoxazolyl)amino]-8-methyl-6-(methylsulfonyl)-3-cinnolinecarboxamide;
4-[(5-chloro-3-pyridinyl)amino]-6-[(1,1-dimethylethyl)sulfonyl]-3-cinnolinecarboxamide;
4-[(5-cyano-3-pyridinyl)amino]-6-[(1,1-dimethylethyl )sulfonyl]-3-cinnolinecarboxamide;
6-[(1,1-dimethylethyl)sulfonyl]-4-[(1-ethyl-1H-pyrazol-5-yl)amino]-3-cinnolinecarboxamide;
4-[(6-fluoro-5-methyl-3-pyridinyl)amino]-8-methyl-6-(methylsulfonyl)-3-cinnolinecarboxamide;
6-(ethylsulfonyl)-4-[(5-fluoro-3-pyridinyl)amino]-8-methyl-3-cinnolinecarboxamide;
4-[(5-cyano-3-pyridinyl)amino]-6-(ethylsulfonyl)-8-methyl-3-cinnolinecarboxamide;
4-{[4-fluoro-3-(methyloxy)phenyl]amino}-8-methyl-6-(methylsulfonyl )-3-cinnolinecarboxamide;
4-[(5-fluoro-3-pyridinyl)amino]-8-methyl-6-(methylsulfonyl)-3-cinnolinecarboxamide;
4-[(5-cyano-3-pyridinyl)amino]-8-methyl-6-(methylsulfonyl)-3-cinnolinecarboxamide;
4-[(1-ethyl-1H-pyrazol-5-yl)amino]-8-methyl-6-(methylsulfonyl)-3-cinnolinecarboxamide;
4-[(2,3-difluorophenyl)amino]-8-methyl-6-(methylsulfonyl)-3-cinnolinecarboxamide;
4-[(3-chloro-2-fluorophenyl)amino]-8-methyl-6-(methylsulfonyl)-3-cinnolinecarboxamide;
4-[(7-fluoro-2,3-dihydro-1-benzofuran-4-yl)amino]-8-methyl-6-(methylsulfonyl)-3-cinnolinecarboxamide;
4-[(3,5-difluorophenyl)amino]-8-methyl-6-(methylsulfonyl)-3-cinnolinecarboxamide;
4-[(1-ethyl-1H-pyrazol-5-yl)amino]-8-methyl-6-(propylsulfonyl )-3-cinnolinecarboxamide;
6-[(1,1-Dimethylethyl)sulfonyl]-4-[(5-fluoro-3-pyridinyl)amino]-8-methyl-3-cinnolinecarboxamide;
4-[(5-Chloro-3-pyridinyl)amino]-6-(ethylsulfonyl)-8-methyl-3-cinnolinecarboxamide;
6-(Ethylsulfonyl)-8-fluoro-4-[(5-fluoro-3-pyridinyl)amino]-3-cinnolinecarboxamide;
8-Fluoro-4-[(5-fluoro-3-pyridinyl)amino]-6-(methylsulfonyl)-3-cinnolinecarboxamide;
4-[(1-Ethyl-1H-pyrazol-5-yl)amino]-6-(ethylsulfonyl)-8-methyl-3-cinnolinecarboxamide;
and their pharmaceutically acceptable salts and solvates thereof.

13. A compound according to claim 12 selected from:
4-[(3-cyanophenyl)amino]-8-methyl-6-(methylsulfonyl)-3-cinnolinecarboxamide;
4-[(3,5-difluorophenyl)amino]-6-(methylsulfonyl)-3-cinnolinecarboxamide;
4-[(5-fluoro-3-pyridinyl)amino]-6-(methylsulfonyl)-3-cinnolinecarboxamide;
6-[(1,1-dimethylethyl)sulfonyl]-4-[(5-fluoro-3-pyridinyl)amino]-3-cinnolinecarboxamide;
4-[(5-chloro-3-pyridinyl)amino]-6-(methylsulfonyl)-3-cinnolinecarboxamide;
4-[(5-chloro-3-pyridinyl)amino]-8-methyl-6-(methylsulfonyl)-3-cinnolinecarboxamide;
4-[(3,4-dimethyl-5-isoxazolyl)amino]-8-methyl-6-(methylsulfonyl)-3-cinnolinecarboxamide;
4-[(1-ethyl-1H-pyrazol-5-yl)amino]-6-(ethylsulfonyl)-8-methyl-3-cinnolinecarboxamide; and pharmaceutically acceptable salts and solvates thereof.

14. A compound according to claim 13 selected from:
4-[(3-Cyanophenyl)amino]-8-methyl-6-(methylsulfonyl)-3-cinnolinecarboxamide
and pharmaceutically acceptable salts and solvates thereof.

15. A compound according to claim 14 which is:
4-[(3-Cyanophenyl)amino]-8-methyl-6-(methylsulfonyl)-3-cinnolinecarboxamide.

16. A method of treatment of diseases or conditions for which a PDE4 inhibitor is indicated, which comprises administering a compound according to claim 1 or a pharmaceutically acceptable salt or solvate thereof.

17. A method according to claim 16 wherein the diseases or conditions for which a PDE4 inhibitor is indicated are inflammatory and/or allergic diseases.

18. A method according to claim 17 wherein the disease is asthma.

19. A method according to claim 17 wherein the disease is COPD.

20. A method according to claim 17 wherein the disease is rheumatoid arthritis.

21.-31. (canceled)

32. A pharmaceutical composition which comprises a compound according to claim 1 or a pharmaceutically acceptable salt or solvate thereof and one or more of pharmaceutically acceptable carriers, deluents and excipients.

33. A pharmaceutical composition according to claim 32 which is suitable for inhaled or administration.

34. A pharmaceutical composition according to claim 32 which is suitable for oral administration.
